SEQUENCE LISTING (80 sequences)

SEQ ID NO: 1 - protein, 735 aa, Adeno-associated dependoparvovirus A [sequence omitted]

SEQ ID NO: 2 - protein, 737 aa, Adeno-associated dependoparvovirus A
  source 1..737: note = Prototypic AAV7 capsid polypeptide
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 3 - protein, 738 aa, Adeno-associated dependoparvovirus A
  source 1..738: note = Prototypic AAV8 capsid polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 4 - protein, 735 aa, synthetic construct
  source 1..735: note = AAVC11.12 capsid polypeptide
MAADGYLPDWLEDTLSEGIREWWALKPGAPQPKANQQHQDNGRGLVLPGYKYLGPFNGLDKGEPVNEADAAALEHDKAYDKQLEQGDNPYLKYNHADAEFQERLQEDTSFGGNLGRAVFQAKKRILEPLGLVEEAAKTAPGKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNYNKSVSVDFTVDTNGVYSEPRPIGTRYLTRNL

SEQ ID NO: 5 - protein, 735 aa, synthetic construct
  source 1..735: note = AAV8-SF capsid polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 6 - protein, 736 aa, synthetic construct
  source 1..736: note = AAV7-SF capsid polypeptide
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 7 - protein, 737 aa, synthetic construct
  source 1..737: note = AAV8-EE capsid polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQSTGGTQGTQQLLFSQAGPANMSAQAKNWLPGPCYRQQRVSTTTGQNNNSNFAWTGATKYHLNGRNSLANPGIAMATHKDDEERFFPSNGILIFGKTGATNKTTLENVLMTNEEEIRPTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 8 - protein, 737 aa, synthetic construct
  source 1..737: note = AAV7-EE capsid polypeptide
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISNGTSGGATNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSTGGTQGTQQLLFSQAGPANMSAQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 9 - protein, 737 aa, synthetic construct
  source 1..737: note = AAV8 Swap16 capsid polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTLSQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 10 - protein, 737 aa, synthetic construct
  source 1..737: note = AAV8 Swap17 capsid polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQSTGGTQGTQQLLFSQAGPANMSAQAKNWLPGPCYRQQRVSTTTGQNNNSNFAWTAGTKYHLNGRNSLANPGIAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 11 - protein, 737 aa, synthetic construct
  source 1..737: note = AAV8 Swap18 capsid polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRNSLVNPGVAMATHKDDEERFFPSNGILIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 12 - protein, 738 aa, synthetic construct
  source 1..738: note = AAV8 Swap19 capsid polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRNSLVNPGVAMATHKDDEDRFFPSSGVLIFGKQNAARDNADYSDVMLTSEEEIKTTNPVATEEYGIVSSNLQAANTAAQTQVVNNQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 13 - protein, 737 aa, synthetic construct
  source 1..737: note = AAV8 Swap20 capsid polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRNSLVNPGVAMATHKDDEDRFFPSSGVLIFGKTGATNKTTLENVLMTNEEEIRPTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 14 - protein, 738 aa, synthetic construct
  source 1..738: note = AAV8 Swap21 capsid polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRNSLVNPGVAMATHKDDEDRFFPSSGVLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 15 - protein, 737 aa, synthetic construct
  source 1..737: note = AAV8 Swap22 capsid polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRNSLVNPGVAMATHKDDEERFFPSNGILIFGKTGATNKTTLENVLMTNEEEIRPTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 16 - protein, 738 aa, synthetic construct
  source 1..738: note = AAV8 Swap23 capsid polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRNSLVNPGVAMATHKDDEERFFPSNGILIFGKQNAARDNADYSDVMLTSEEEIKTTNPVATEEYGIVSSNLQAANTAAQTQVVNNQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 17 - DNA, 2211 bp, Adeno-associated dependoparvovirus A
  source 1..2211: mol_type = genomic DNA; note = AAV7 capsid polynucleotide
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctg

SEQ ID NO: 18 - DNA, 2217 bp, Adeno-associated dependoparvovirus A
  source 1..2217: mol_type = genomic DNA; note = AAV8 capsid polynucleotide
atggctgccgatggttatcttccagattggctcgaggacaacctctctgagggcattcgcgagtggtgggcgctgaaacctggagccccgaagcccaaagccaaccagcaaaagcaggacgacggccggggtctagtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 19 - DNA, 2208 bp, synthetic construct
  source 1..2208: mol_type = other DNA; note = AAVC11.12 capsid polynucleotide
atggctgccgatggttatcttccagattggctcgaggacactctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagctgctaagacggctcctggaaagaagagaccggtagaaccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcatcaccaccagcaccagaacctgggccctgcccacttacaacaaccatctctacaagcaaatctccagccaatcaggagcttcaaacgacaaccactacttcggctacagcaccccctgggggtattttgactttaacagattccactgccacttttcaccacgtgactggcagcgactcatcaacaacaactggggattccggcccaagagactcagcttcaagctcttcaacatccaagtcaaggaggtcacgacgaatgatggcgtcacgaccatcgctaataaccttaccagcacggttcaagtcttctcggactcggagtaccagcttccgtacgtcctcggctctgcgcaccagggctgcctccctccgttcccggcggacgtgttcatgattccgcagtacggctacctaacgctcaacaatggcagccaggcagtgggacggtcatccttttactgcctggaatattttccatctcaaatgctgcgaactggaaacaattttgaattcagctacaccttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacttcaaactacaacaagtctgttagtgtggactttactgtagacactaatggcgtgtattcagagcctcgccccattggcaccagatacctgactcgtaatctgtaa

SEQ ID NO: 20 - DNA, 2208 bp, synthetic construct
  source 1..2208: mol_type = other DNA; note = AAV8-SF capsid polynucleotide
atggctgccgatggttatcttccagattggctcgaggacaacctctctgagggcattcgcgagtggtgggcgctgaaacctggagccccgaagcccaaagccaaccagcaaaagcaggacgacggccggggtctagtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcaccagaacctgggccctgcccacttacaacaaccatctctacaagcaaatctccagccaatcaggagcttcaaacgacaaccactacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaggacgacgaggagcgctttttcccatccaacggaatcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 21 - DNA, 2211 bp, synthetic construct
  source 1..2211: mol_type = other DNA; note = AAV7-SF capsid polynucleotide
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcatcaccaccagcaccagaacctgggccctgcccacttacaacaaccatctctacaagcaaatctccagccaatcaggagcttcaaacgacaaccactacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaa

SEQ ID NO: 22 - DNA, 2208 bp, synthetic construct
  source 1..2208: mol_type = other DNA; note = AAV8-EE capsid polynucleotide
atggctgccgatggttatcttccagattggctcgaggacaacctctctgagggcattcgcgagtggtgggcgctgaaacctggagccccgaagcccaaagccaaccagcaaaagcaggacgacggccggggtctagtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcaccagaacctgggccctgcccacttacaacaaccatctctacaagcaaatctccagccaatcaggagcttcaaacgacaaccactacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacaacggggcaaaacaacaacagcaactttgcttggactggtgccaccaaatatcacctgaacggcagaaactcgttggctaatcccggcatcgccatggcaacacacaaggacgacgaggagcgtttttttcccagtaacgggatcctgatttttggcaaaactggtgccacaaacaaaacgactttggagaatgtcttgatgaccaacgaggaagaaatcagacccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 23 - DNA, 2214 bp, synthetic construct
  source 1..2214: mol_type = other DNA; note = AAV7-EE capsid polynucleotide
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaactcagtccacaggaggaactcaaggtacccagcaattgttattttctcaagctgggcctgcaaacatgtcggctcaggccaagaactggctgcctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaa

SEQ ID NO: 24 - DNA, 2214 bp, synthetic construct
  source 1..2214: mol_type = other DNA; note = AAV8 Swap16 capsid polynucleotide
atggctgccgatggttatcttccagattggctcgaggacaacctctctgagggcattcgcgagtggtgggcgctgaaacctggagccccgaagcccaaagccaaccagcaaaagcaggacgacggccggggtctagtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcaccagaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttatccagaactcagaccacaggaggaactgcaaatacccagacattgggattttctcaaggtgggcctaacaccatggcgaatcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaacacacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 25 - DNA, 2214 bp, synthetic construct
  source 1..2214: mol_type = other DNA; note = AAV8 Swap17 capsid polynucleotide
atggctgccgatggttatcttccagattggctcgaggacaacctctctgagggcattcgcgagtggtgggcgctgaaacctggagccccgaagcccaaagccaaccagcaaaagcaggacgacggccggggtctagtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacaacggggcaaaacaacaacagcaactttgcttggactgctggcaccaaatatcacctgaacggcagaaactcgttggctaatcccggcatcgccatggcaacacacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 26 - DNA, 2214 bp, synthetic construct
  source 1..2214: mol_type = other DNA; note = AAV8 Swap18 capsid polynucleotide
atggctgccgatggttatcttccagattggctcgaggacaacctctctgagggcattcgcgagtggtgggcgctgaaacctggagccccgaagcccaaagccaaccagcaaaagcaggacgacggccggggtctagtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaacacacaaggacgacgaggagcgctttttcccatccaacggaatcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 27 - DNA, 2217 bp, synthetic construct
  source 1..2217: mol_type = other DNA; note = AAV8 Swap19 capsid polynucleotide
atggctgccgatggttatcttccagattggctcgaggacaacctctctgagggcattcgcgagtggtgggcgctgaaacctggagccccgaagcccaaagccaaccagcaaaagcaggacgacggccggggtctagtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaacacacaaggacgacgaggaccgctttttcccatccagcggagtcctgatttttggaaaacagaatgcagcaagggacaacgctgactactcagatgtgatgttgacaagtgaagaagaaattaagactactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 28 - DNA, 2214 bp, synthetic construct
  source 1..2214: mol_type = other DNA; note = AAV8 Swap20 capsid polynucleotide
atggctgccgatggttatcttccagattggctcgaggacaacctctctgagggcattcgcgagtggtgggcgctgaaacctggagccccgaagcccaaagccaaccagcaaaagcaggacgacggccggggtctagtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaacacacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcgccgacaacttacaacagcagaatactgcaccccagataggaactgtcaacagccagggagccttacctggcatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 29 - DNA, 2217 bp, synthetic construct
  source 1..2217: mol_type = other DNA; note = AAV8 Swap21 capsid polynucleotide
atggctgccgatggttatcttccagattggctcgaggacaacctctctgagggcattcgcgagtggtgggcgctgaaacctggagccccgaagcccaaagccaaccagcaaaagcaggacgacggccggggtctagtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaacacacaaggacgacgaggaccgtttttttcccagtagcggggtcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 30 - DNA, 2214 bp, synthetic construct
  source 1..2214: mol_type = other DNA; note = AAV8 Swap22 capsid polynucleotide
atggctgccgatggttatcttccagattggctcgaggacaacctctctgagggcattcgcgagtggtgggcgctgaaacctggagccccgaagcccaaagccaaccagcaaaagcaggacgacggccggggtctagtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaacacacaaggacgacgaggagcgtttttttcccagtaacgggatcctgatttttggcaaaactggtgccacaaacaaaacgactttggagaatgtcttgatgaccaacgaggaagaaatcagacccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 31 - DNA, 2217 bp, synthetic construct
  source 1..2217: mol_type = other DNA; note = AAV8 Swap23 capsid polynucleotide
atggctgccgatggttatcttccagattggctcgaggacaacctctctgagggcattcgcgagtggtgggcgctgaaacctggagccccgaagcccaaagccaaccagcaaaagcaggacgacggccggggtctagtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaacacacaaggacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtgtcatctaacttgcaggcggcaaacacggctgctcaaactcaagttgtcaacaaccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 32 - protein, 10 aa, synthetic construct
SQSGASNDNH

SEQ ID NO: 33 - protein, 22 aa, synthetic construct
TGATNKTTLENVLMTNEEEIRP

SEQ ID NO: 34 - protein, 18 aa, synthetic construct
SSNLQAANTAAQTQVVNN

SEQ ID NO: 35 - protein, 9 aa, synthetic construct
DRFFPSSGV

SEQ ID NO: 36 - protein, 23 aa, synthetic construct
STGGTQGTQQLLFSQAGPANMSA

SEQ ID NO: 37 - protein, 30 aa, synthetic construct
LSQNNNSNFAWTGATKYHLNGRNSLVNPGV

SEQ ID NO: 38 - protein, 12 aa, synthetic construct
ISSQSGASNDNH

SEQ ID NO: 39 - protein, 23 aa, synthetic construct
KTGATNKTTLENVLMTNEEEIRP

SEQ ID NO: 40 - protein, 18 aa, synthetic construct
AMATHKDDEDRFFPSSGV

SEQ ID NO: 41 - protein, 24 aa, synthetic construct
QSTGGTQGTQQLLFSQAGPANMSA

SEQ ID NO: 42 - protein, 35 aa, synthetic construct
RVSTTLSQNNNSNFAWTGATKYHLNGRNSLVNPGV

SEQ ID NO: 43 - protein, 9 aa, synthetic construct
ERFFPSNGI

SEQ ID NO: 44 - protein, 12 aa, synthetic construct
NGTSGGATNDNT

SEQ ID NO: 45 - protein, 18 aa, synthetic construct
AMATHKDDEERFFPSNGI

SEQ ID NO: 46 - protein, 14 aa, synthetic construct
ISNGTSGGATNDNT

SEQ ID NO: 47 - protein, 735 aa, synthetic construct
  source 1..735: note = AAV C11.12.253R4.1 capsid polypeptide
MAADGYLPDWLEDTLSEGIREWWALKPGAPQPKANQQHQDNGRGLVLPGYKYLGPFNGLDKGEPVNEADAAALEHDKAYDKQLEQGDNPYLKYNHADAEFQERLQEDTSFGGNLGRAVFQAKKRILEPLGLVEEAAKTAPGKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRNSLVNPGVAMATHKDDEDRFFPSSGVLIFGKQGSANDATHEDVMLTDEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNYNKSVSVDFTVDTNGVYSEPRPIGTRYLTRNL

SEQ ID NO: 48 - protein, 735 aa, synthetic construct
  source 1..735: note = AAV C11.12.253R4.3 capsid polypeptide
MAADGYLPDWLEDTLSEGIREWWALKPGAPQPKANQQHQDNGRGLVLPGYKYLGPFNGLDKGEPVNEADAAALEHDKAYDKQLEQGDNPYLKYNHADAEFQERLQEDTSFGGNLGRAVFQAKKRILEPLGLVEEAAKTAPGKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRNSLVNPGVAMATHKDDEDRFFPSSGVLIFGKTGATNKTTLENVLMTNEEEIRPTNPVATEEYGIVSSNLQNSNTAPEIANVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNYNKSVSVDFTVDTNGVYSEPRPIGTRYLTRNL

SEQ ID NO: 49 - protein, 735 aa, synthetic construct
  source 1..735: note = AAV C11.12.253R4.6 capsid polypeptide
MAADGYLPDWLEDTLSEGIREWWALKPGAPQPKANQQHQDNGRGLVLPGYKYLGPFNGLDKGEPVNEADAAALEHDKAYDKQLEQGDNPYLKYNHADAEFQERLQEDTSFGGNLGRAVFQAKKRILEPLGLVEEAAKTAPGKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRNSLVNPGVAMATHKDDEDRFFPSSGVLIFGKQGSANDATHEDVMLTDEEEIRPTNPVATEEYGIVSSNLQNSNTAPEIANVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNYNKSVSVDFTVDTNGVYSEPRPIGTRYLTRNL

SEQ ID NO: 50 - protein, 736 aa, synthetic construct
  source 1..736: note = AAV C11.12.253R4.9 capsid polypeptide
MAADGYLPDWLEDTLSEGIREWWALKPGAPQPKANQQHQDNGRGLVLPGYKYLGPFNGLDKGEPVNEADAAALEHDKAYDKQLEQGDNPYLKYNHADAEFQERLQEDTSFGGNLGRAVFQAKKRILEPLGLVEEAAKTAPGKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRNSLVNPGVAMATHKDDEDRFFPSSGVLIFGKKNTNTNNVNYENVMMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNYNKSVSVDFTVDTNGVYSEPRPIGTRYLTRNL

SEQ ID NO: 51 - protein, 736 aa, synthetic construct
  source 1..736: note = AAV C11.12.269R4.1 capsid polypeptide
MAADGYLPDWLEDTLSEGIREWWALKPGAPQPKANQQHQDNGRGLVLPGYKYLGPFNGLDKGEPVNEADAAALEHDKAYDKQLEQGDNPYLKYNHADAEFQERLQEDTSFGGNLGRAVFQAKKRILEPLGLVEEAAKTAPGKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLGRTQATPGGTTGQNTLLFSQAGPANMSAQAKNWLPGPCYRQQRVSTTLSQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNYNKSVSVDFTVDTNGVYSEPRPIGTRYLTRNL

SEQ ID NO: 52 - protein, 735 aa, synthetic construct
  source 1..735: note = AAV C11.12.269R4.3 capsid polypeptide
MAADGYLPDWLEDTLSEGIREWWALKPGAPQPKANQQHQDNGRGLVLPGYKYLGPFNGLDKGEPVNEADAAALEHDKAYDKQLEQGDNPYLKYNHADAEFQERLQEDTSFGGNLGRAVFQAKKRILEPLGLVEEAAKTAPGKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRNSLVNPGVAMATHKDDEDRFFPSSGVLIFGKTGATNKTTLENVLMTNEEEIRPTNPVATEEYGIVSSNLQKENTPAQIEAVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNYNKSVSVDFTVDTNGVYSEPRPIGTRYLTRNL

SEQ ID NO: 53 - protein, 736 aa, synthetic construct
  source 1..736: note = AAV C11.12.269R4.5 capsid polypeptide
MAADGYLPDWLEDTLSEGIREWWALKPGAPQPKANQQHQDNGRGLVLPGYKYLGPFNGLDKGEPVNEADAAALEHDKAYDKQLEQGDNPYLKYNHADAEFQERLQEDTSFGGNLGRAVFQAKKRILEPLGLVEEAAKTAPGKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRNSLVNPGVAMATHKDDEDRFFPSSGVLIFGKQSSSGAKVNLEGVLLTGEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNYNKSVSVDFTVDTNGVYSEPRPIGTRYLTRNL

SEQ ID NO: 54 - protein, 735 aa, synthetic construct
  source 1..735: note = AAV C11.12.269R4.6 capsid polypeptide
MAADGYLPDWLEDTLSEGIREWWALKPGAPQPKANQQHQDNGRGLVLPGYKYLGPFNGLDKGEPVNEADAAALEHDKAYDKQLEQGDNPYLKYNHADAEFQERLQEDTSFGGNLGRAVFQAKKRILEPLGLVEEAAKTAPGKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSKTQATSGTKDTNQLLFSQAGPANMSAQAKNWLPGPCYRQQRVSTTLSQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNYNKSVSVDFTVDTNGVYSEPRPIGTRYLTRNL

SEQ ID NO: 55 - protein, 735 aa, synthetic construct
  source 1..735: note = AAV C11.12.269R4.20 capsid polypeptide
MAADGYLPDWLEDTLSEGIREWWALKPGAPQPKANQQHQDNGRGLVLPGYKYLGPFNGLDKGEPVNEADAAALEHDKAYDKQLEQGDNPYLKYNHADAEFQERLQEDTSFGGNLGRAVFQAKKRILEPLGLVEEAAKTAPGKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRNSLVNPGVAMATHKDDEDRFFPSSGVLIFGKTGATNKTTLENVLMTNEEEIRPTNPVATEEYGIVSSNLQTGNTAPETLIVNHQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNYNKSVSVDFTVDTNGVYSEPRPIGTRYLTRNL

SEQ ID NO: 56 - DNA, 2208 bp, synthetic construct
  source 1..2208: mol_type = other DNA; note = AAV C11.12.253R4.1 capsid polynucleotide
atggctgccgatggttatcttccagattggctcgaggacactctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagctgctaagacggctcctggaaagaagagaccggtagaaccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcatcaccaccagcaccagaacctgggccctgcccacttacaacaaccatctctacaagcaaatctccagccaatcaggagcttcaaacgacaaccactacttcggctacagcaccccctgggggtattttgactttaacagattccactgccacttttcaccacgtgactggcagcgactcatcaacaacaactggggattccggcccaagagactcagcttcaagctcttcaacatccaagtcaaggaggtcacgacgaatgatggcgtcacgaccatcgctaataaccttaccagcacggttcaagtcttctcggactcggagtaccagcttccgtacgtcctcggctctgcgcaccagggctgcctccctccgttcccggcggacgtgttcatgattccgcagtacggctacctaacgctcaacaatggcagccaggcagtgggacggtcatccttttactgcctggaatattttccatctcaaatgctgcgaactggaaacaattttgaattcagctacaccttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaactcacaaggacgacgaggaccgctttttcccatccagcggggtactaatcttcggtaaacagggctcggccaacgacgcgacccacgaggacgtgatgctcacagacgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacttcaaactacaacaagtctgttagtgtggactttactgtagacactaatggcgtgtattcagagcctcgccccattggcaccagatacctgactcgtaatctgtaa

SEQ ID NO: 57 - DNA, 2208 bp, synthetic construct
  source 1..2208: mol_type = other DNA; note = AAV C11.12.253R4.3 capsid polynucleotide
atggctgccgatggttatcttccagattggctcgaggacactctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagctgctaagacggctcctggaaagaagagaccggtagaaccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcatcaccaccagcaccagaacctgggccctgcccacttacaacaaccatctctacaagcaaatctccagccaatcaggagcttcaaacgacaaccactacttcggctacagcaccccctgggggtattttgactttaacagattccactgccacttttcaccacgtgactggcagcgactcatcaacaacaactggggattccggcccaagagactcagcttcaagctcttcaacatccaagtcaaggaggtcacgacgaatgatggcgtcacgaccatcgctaataaccttaccagcacggttcaagtcttctcggactcggagtaccagcttccgtacgtcctcggctctgcgcaccagggctgcctccctccgttcccggcggacgtgttcatgattccgcagtacggctacctaacgctcaacaatggcagccaggcagtgggacggtcatccttttactgcctggaatattttccatctcaaatgctgcgaactggaaacaattttgaattcagctacaccttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaaaacagcaatactgcgccggaaatcgcaaacgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacttcaaactacaacaagtctgttagtgtggactttactgtagacactaatggcgtgtattcagagcctcgccccattggcaccagatacctgactcgtaatctgtaa

SEQ ID NO: 58 - DNA, 2208 bp, synthetic construct
  source 1..2208: mol_type = other DNA; note = AAV C11.12.253R4.6 capsid polynucleotide
atggctgccgatggttatcttccagattggctcgaggacactctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagctgctaagacggctcctggaaagaagagaccggtagaaccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcatcaccaccagcaccagaacctgggccctgcccacttacaacaaccatctctacaagcaaatctccagccaatcaggagcttcaaacgacaaccactacttcggctacagcaccccctgggggtattttgactttaacagattccactgccacttttcaccacgtgactggcagcgactcatcaacaacaactggggattccggcccaagagactcagcttcaagctcttcaacatccaagtcaaggaggtcacgacgaatgatggcgtcacgaccatcgctaataaccttaccagcacggttcaagtcttctcggactcggagtaccagcttccgtacgtcctcggctctgcgcaccagggctgcctccctccgttcccggcggacgtgttcatgattccgcagtacggctacctaacgctcaacaatggcagccaggcagtgggacggtcatccttttactgcctggaatattttccatctcaaatgctgcgaactggaaacaattttgaattcagctacaccttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaactcacaaggacgacgaggaccgctttttcccatccagcggggtactaatcttcggtaaacagggctcggccaacgacgcgacccacgaggacgtgatgctcacagacgaagaagaaattcgtcctactaatcctgtagccacggaagaatacgggatagtcagcagcaacttacaaaacagcaatactgcgccggaaatcgcaaacgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacttcaaactacaacaagtctgttagtgtggactttactgtagacactaatggcgtgtattcagagcctcgccccattggcaccagatacctgactcgtaatctgtaa

SEQ ID NO: 59 - DNA, 2211 bp, synthetic construct
  source 1..2211: mol_type = other DNA; note = AAV C11.12.253R4.9 capsid polynucleotide
atggctgccgatggttatcttccagattggctcgaggacactctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagctgctaagacggctcctggaaagaagagaccggtagaaccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcatcaccaccagcaccagaacctgggccctgcccacttacaacaaccatctctacaagcaaatctccagccaatcaggagcttcaaacgacaaccactacttcggctacagcaccccctgggggtattttgactttaacagattccactgccacttttcaccacgtgactggcagcgactcatcaacaacaactggggattccggcccaagagactcagcttcaagctcttcaacatccaagtcaaggaggtcacgacgaatgatggcgtcacgaccatcgctaataaccttaccagcacggttcaagtcttctcggactcggagtaccagcttccgtacgtcctcggctctgcgcaccagggctgcctccctccgttcccggcggacgtgttcatgattccgcagtacggctacctaacgctcaacaatggcagccaggcagtgggacggtcatccttttactgcctggaatattttccatctcaaatgctgcgaactggaaacaattttgaattcagctacaccttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaactcacaaggacgacgaggaccgctttttcccatccagcggagttctaatctttggtaagaagaacacgaacacgaacaacgtgaactacgagaacgtgatgatgacaaacgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacttcaaactacaacaagtctgttagtgtggactttactgtagacactaatggcgtgtattcagagcctcgccccattggcaccagatacctgactcgtaatctgtaa

SEQ ID NO: 60 - DNA, 2211 bp, synthetic construct
  source 1..2211: mol_type = other DNA; note = AAV C11.12.269R4.1 capsid polynucleotide
atggctgccgatggttatcttccagattggctcgaggacactctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagctgctaagacggctcctggaaagaagagaccggtagaaccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcatcaccaccagcaccagaacctgggccctgcccacttacaacaaccatctctacaagcaaatctccagccaatcaggagcttcaaacgacaaccactacttcggctacagcaccccctgggggtattttgactttaacagattccactgccacttttcaccacgtgactggcagcgactcatcaacaacaactggggattccggcccaagagactcagcttcaagctcttcaacatccaagtcaaggaggtcacgacgaatgatggcgtcacgaccatcgctaataaccttaccagcacggttcaagtcttctcggactcggagtaccagcttccgtacgtcctcggctctgcgcaccagggctgcctccctccgttcccggcggacgtgttcatgattccgcagtacggctacctaacgctcaacaatggcagccaggcagtgggacggtcatccttttactgcctggaatattttccatctcaaatgctgcgaactggaaacaattttgaattcagctacaccttcgaggacgtgcctttccacagcagctacgcacacagccagagcttggaccgactgatgaatcctctcattgaccaatatctatactatttaggcaggactcaggcgaccccgggcggaactacggggcagaacaccttgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacttcaaactacaacaagtctgttagtgtggactttactgtagacactaatggcgtgtattcagagcctcgccccattggcaccagatacctgactcgtaatctgtaa

SEQ ID NO: 61 - DNA, 2208 bp, synthetic construct
  source 1..2208: mol_type = other DNA; note = AAV C11.12.269R4.3 capsid polynucleotide
atggctgccgatggttatcttccagattggctcgaggacactctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagctgctaagacggctcctggaaagaagagaccggtagaaccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcatcaccaccagcaccagaacctgggccctgcccacttacaacaaccatctctacaagcaaatctccagccaatcaggagcttcaaacgacaaccactacttcggctacagcaccccctgggggtattttgactttaacagattccactgccacttttcaccacgtgactggcagcgactcatcaacaacaactggggattccggcccaagagactcagcttcaagctcttcaacatccaagtcaaggaggtcacgacgaatgatggcgtcacgaccatcgctaataaccttaccagcacggttcaagtcttctcggactcggagtaccagcttccgtacgtcctcggctctgcgcaccagggctgcctccctccgttcccggcggacgtgttcatgattccgcagtacggctacctaacgctcaacaatggcagccaggcagtgggacggtcatccttttactgcctggaatattttccatctcaaatgctgcgaactggaaacaattttgaattcagctacaccttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaaaaggagaatactccggcgcaaatcgaagccgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacttcaaactacaacaagtctgttagtgtggactttactgtagacactaatggcgtgtattcagagcctcgccccattggcaccagatacctgactcgtaatctgtaa

SEQ ID NO: 62 - DNA, 2211 bp, synthetic construct
  source 1..2211: mol_type = other DNA; note = AAV C11.12.269R4.5 capsid polynucleotide
atggctgccgatggttatcttccagattggctcgaggacactctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagctgctaagacggctcctggaaagaagagaccggtagaaccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcatcaccaccagcaccagaacctgggccctgcccacttacaacaaccatctctacaagcaaatctccagccaatcaggagcttcaaacgacaaccactacttcggctacagcaccccctgggggtattttgactttaacagattccactgccacttttcaccacgtgactggcagcgactcatcaacaacaactggggattccggcccaagagactcagcttcaagctcttcaacatccaagtcaaggaggtcacgacgaatgatggcgtcacgaccatcgctaataaccttaccagcacggttcaagtcttctcggactcggagtaccagcttccgtacgtcctcggctctgcgcaccagggctgcctccctccgttcccggcggacgtgttcatgattccgcagtacggctacctaacgctcaacaatggcagccaggcagtgggacggtcatccttttactgcctggaatattttccatctcaaatgctgcgaactggaaacaattttgaattcagctacaccttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaactcacaaggacgacgaggaccgctttttcccatccagcggggtcctgatatttggtaagcagagctcgagcggggccaaggtgaacctcgagggggtgttgctcacaggcgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacttcaaactacaacaagtctgttagtgtggactttactgtagacactaatggcgtgtattcagagcctcgccccattggcaccagatacctgactcgtaatctgtaa

SEQ ID NO: 63 - DNA, 2208 bp, synthetic construct
  source 1..2208: mol_type = other DNA; note = AAV C11.12.269R4.6 capsid polynucleotide
atggctgccgatggttatcttccagattggctcgaggacactctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagctgctaagacggctcctggaaagaagagaccggtagaaccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcatcaccaccagcaccagaacctgggccctgcccacttacaacaaccatctctacaagcaaatctccagccaatcaggagcttcaaacgacaaccactacttcggctacagcaccccctgggggtattttgactttaacagattccactgccacttttcaccacgtgactggcagcgactcatcaacaacaactggggattccggcccaagagactcagcttcaagctcttcaacatccaagtcaaggaggtcacgacgaatgatggcgtcacgaccatcgctaataaccttaccagcacggttcaagtcttctcggactcggagtaccagcttccgtacgtcctcggctctgcgcaccagggctgcctccctccgttcccggcggacgtgttcatgattccgcagtacggctacctaacgctcaacaatggcagccaggcagtgggacggtcatccttttactgcctggaatattttccatctcaaatgctgcgaactggaaacaattttgaattcagctacaccttcgaggacgtgcctttccacagcagctacgcacacagccagagcttggaccgactgatgaatcctctcattgaccagtacctgtactacttaagcaagactcaggcgaccagcggaactaaggacaccaaccagttgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacttcaaactacaacaagtctgttagtgtggactttactgtagacactaatggcgtgtattcagagcctcgccccattggcaccagatacctgactcgtaatctgtaa

SEQ ID NO: 64 - DNA, 2208 bp, synthetic construct
  source 1..2208: mol_type = other DNA; note = AAV C11.12.269R4.20 capsid polynucleotide
atggctgccgatggttatcttccagattggctcgaggacactctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagctgctaagacggctcctggaaagaagagaccggtagaaccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcatcaccaccagcaccagaacctgggccctgcccacttacaacaaccatctctacaagcaaatctccagccaatcaggagcttcaaacgacaaccactacttcggctacagcaccccctgggggtattttgactttaacagattccactgccacttttcaccacgtgactggcagcgactcatcaacaacaactggggattccggcccaagagactcagcttcaagctcttcaacatccaagtcaaggaggtcacgacgaatgatggcgtcacgaccatcgctaataaccttaccagcacggttcaagtcttctcggactcggagtaccagcttccgtacgtcctcggctctgcgcaccagggctgcctccctccgttcccggcggacgtgttcatgattccgcagtacggctacctaacgctcaacaatggcagccaggcagtgggacggtcatccttttactgcctggaatattttccatctcaaatgctgcgaactggaaacaattttgaattcagctacaccttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaaacggggaatactgcgccggaaaccctaatcgtcaaccaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacttcaaactacaacaagtctgttagtgtggactttactgtagacactaatggcgtgtattcagagcctcgccccattggcaccagatacctgactcgtaatctgtaa

SEQ ID NO: 65 - protein, 16 aa, synthetic construct
QGSANDATHEDVMLTD

SEQ ID NO: 66 - protein, 23 aa, synthetic construct
KQGSANDATHEDVMLTDEEEIRP

SEQ ID NO: 67 - protein, 10 aa, synthetic construct
NSNTAPEIAN

SEQ ID NO: 68 - protein, 18 aa, synthetic construct
SSNLQNSNTAPEIANVNN

SEQ ID NO: 69 - protein, 15 aa, synthetic construct
KNTNTNNVNYENVMM

SEQ ID NO: 70 - protein, 24 aa, synthetic construct
KKNTNTNNVNYENVMMTNEEEIRP

SEQ ID NO: 71 - protein, 15 aa, synthetic construct
GRTQATPGGTTGQNT

SEQ ID NO: 72 - protein, 28 aa, synthetic construct
GRTQATPGGTTGQNTLLFSQAGPANMSA

SEQ ID NO: 73 - protein, 10 aa, synthetic construct
KENTPAQIEA

SEQ ID NO: 74 - protein, 19 aa, synthetic construct
SSNLQKENTPAQIEAVNNQ

SEQ ID NO: 75 - protein, 17 aa, synthetic construct
QSSSGAKVNLEGVLLTG

SEQ ID NO: 76 - protein, 24 aa, synthetic construct
KQSSSGAKVNLEGVLLTGEEEIRP

SEQ ID NO: 77 - protein, 12 aa, synthetic construct
KTQATSGTKDTN

SEQ ID NO: 78 - protein, 26 aa, synthetic construct
KTQATSGTKDTNQLLFSQAGPANMSA

SEQ ID NO: 79 - protein, 13 aa, synthetic construct
TGNTAPETLIVNH

SEQ ID NO: 80 - protein, 18 aa, synthetic construct
SSNLQTGNTAPETLIVNH

(19) United States
(12) Patent Application Publication
(10) Pub. No.: US 2024/0425549 A1
(43) Pub. Date: Dec. 26, 2024

(54) MODIFIED AAV CAPSID POLYPEPTIDES AND VECTORS

(71) Applicants: Children's Medical Research Institute, Westmead (AU); The Sydney Children's Hospitals Network (Randwick and Westmead) (Incorporating the Royal Alexandra H, Westmead (AU); Logicbio Therapeutics, Inc., Lexington, MA (US)

(72) Inventors: Marti Cabanes Creus, Westmead (AU); Leszek Lisowski, Westmead (AU); Ian Alexander, Pennant Hills (AU); Matthias Charles Jerome Hebben, Lexington, MA (US)

(21) Appl. No.: 18/686,145

(22) PCT Filed: Aug. 25, 2022
(86) PCT No.: PCT/AU2022/051008

§ 371 Date: Feb. 23, 2024

(30) Foreign Application Priority Data
Aug. 25, 2021 (AU) 2021902729

(51) Int. Cl. C07K 14/005

(52) U.S. Cl. CPC C07K 14/005; C12N 2750/14122

(57) ABSTRACT

The present disclosure relates generally to modified adeno-associated virus (AAV) capsid polypeptides and encoding nucleic acid molecules. The disclosure also relates to AAV vectors comprising the capsid polypeptides, and nucleic acid vectors (e.g. plasmids) comprising the encoding nucleic acids molecules, as well as to host cells comprising the vectors. The disclosure also relates to methods and uses of the polypeptides, encoding nucleic acids molecules, vectors and host cells.

### FIELD OF THE DISCLOSURE

The present disclosure relates generally to modified adeno-associated virus (AAV) capsid polypeptides and encoding nucleic acid molecules. The disclosure also relates to AAV vectors comprising the capsid polypeptides, and nucleic acid vectors (e.g. plasmids) comprising the encoding nucleic acids molecules, as well as to host cells comprising the vectors. The disclosure also relates to methods and uses of the polypeptides, encoding nucleic acids molecules, vectors and host cells.

### BACKGROUND OF THE DISCLOSURE

Gene therapy has most commonly been investigated and achieved using viral vectors, with notable recent advances being based on adeno-associated viral vectors. Adeno-associated virus (AAV) is a replication-deficient parvovirus, the single-stranded DNA genome of which is about 4.7 kb in length. The AAV genome includes inverted terminal repeat (ITRs) at both ends of the molecule, flanking two open reading frames: rep and cap. The cap gene encodes three structural capsid proteins: VP1, VP2 and VP3. The three capsid proteins typically assemble in a ratio of 1:1:8-10 to form the AAV capsid, although AAV capsids containing only VP3, or VP1 and VP3, or VP2 and VP3, have been produced. The cap gene also encodes the assembly activating protein (AAP) from an alternative open reading frame. AAP promotes capsid assembly, acting to target the capsid proteins to the nucleolus and promote capsid formation. The rep gene encodes four known regulatory proteins: Rep78, Rep68, Rep52 and Rep40. These Rep proteins are involved in AAV genome replication, packaging, genomic integration and other processes. More recently, an X gene has been identified in the 3′ end of the AAV2 genome (Cao et al. PLoS One, 2014, 9:e104596). The encoded X protein appears to be involved in the AAV life cycle, including DNA replication.

The ITRs are involved in several functions, in particular integration of the AAV DNA into the host cell genome, as well as genome replication and packaging. When AAV infects a host cell, the viral genome can integrate into the host's chromosomal DNA resulting in latent infection of the cell. Thus, AAV can be exploited to introduce heterologous sequences into cells. In nature, a helper virus (for example, adenovirus or herpesvirus) provides protein factors that allow for replication of AAV virus in the infected cell and packaging of new virions. In the case of adenovirus, genes E1A, E1B, E2A, E4 and VA provide helper functions. Upon infection with a helper virus, the AAV provirus is rescued and amplified, and both AAV and the helper virus are produced.

AAV vectors (also referred to as recombinant AAV, rAAV) that contain a genome that lacks some, most or all of the native AAV genome and instead contain one or more heterologous sequences flanked by the ITRs, have been successfully used in gene therapy settings. These AAV vectors are widely used to deliver heterologous nucleic acid to cells of a subject for therapeutic purposes, and in many instances, it is the expression of the heterologous nucleic acid that imparts the therapeutic effect. Although several AAV vectors have now been used in the clinic, there are a limited number that exhibit the required in vivo transduction efficiency of primary human cells/tissues to facilitate adequate expression of the heterologous nucleic acid for therapeutic applications. There is therefore a need to develop alternative AAV vectors that contain capsid proteins that facilitate efficient transduction of host cells in vivo.

### SUMMARY OF THE DISCLOSURE

The present disclosure is predicated in part on the generation of modified AAV capsid polypeptides. In particular embodiments, the capsid polypeptides facilitate efficient transduction of human cells (such as human hepatocytes) when contained in an AAV vector. Typically, the in vivo transduction of AAV vectors comprising a capsid polypeptide of the present disclosure is improved or enhanced compared to AAV vectors comprising other AAV capsid polypeptides (e.g. the prototypic AAV2 capsid set forth in SEQ ID NO:1). The capsid polypeptides of the present disclosure are therefore particularly useful in preparing AAV vectors, and in particular, AAV vectors for gene therapy uses. Similarly, AAV vectors comprising a capsid polypeptide of the present disclosure (i.e. having a capsid comprising or consisting of a capsid polypeptide of the present disclosure) are of particular use in gene therapy applications, such as for delivery of heterologous nucleic acids for the treatment of various diseases and conditions.

In one aspect, the disclosure provides a capsid polypeptide, comprising: (i) the sequence of amino acids set forth in any one of SEQ ID NOs:5-8, or a sequence having at least or about 95%, 96%, 97%, 98% or 99% sequence identity thereto; (ii) the sequence of amino acids at positions 138-735 of SEQ ID NO:5, positions 138-735 of SEQ ID NO:6, or positions 138-737 of SEQ ID NO:7 or 8; or a sequence having at least or about 95%, 96%, 97%, 98% or 99% sequence identity thereto; and/or (iii) the sequence of amino acids at positions 204-735 of SEQ ID NO:5, positions 204-735 of SEQ ID NO:6, or positions 204-737 of SEQ ID NO: 7 or 8, or a sequence having at least or about 95%, 96%, 97%, 98% or 99% sequence identity thereto.

In another aspect, provided is a capsid polypeptide, comprising: (i) the sequence of amino acids set forth in SEQ ID NO:5 or a sequence having at least or about 85% sequence identity thereto; (ii) the sequence of amino acids at positions 138-735 of SEQ ID NO:5 or a sequence having at least or about 85% sequence identity thereto; and/or (iii) the sequence of amino acids at positions 204-735 of SEQ ID NO:5 or a sequence having at least or about 85% sequence identity thereto; wherein the capsid polypeptide comprises: a) amino acid residues S263, Q264, S265, S268 and H272, with numbering relative to SEQ ID NO:5; b) amino acid residues E532, N538 and 1540, with numbering relative to SEQ ID NO:5 c) amino acid residues T546, G547, T549, N550, K551, T552, T553, L554, E555, N556, L558, M559, N561, R566 and P567, with numbering relative to SEQ ID NO:5; and d) amino acid residues S580, S581, A585, A586, A590, T592, Q593, V594, and N597, with numbering relative to SEQ ID NO:5. In some examples, the capsid polypeptide comprises a) the sequence of amino acids SQSGASNDNH (SEQ ID NO:32) at positions 263-272, with numbering relative to SEQ ID NO:5; b) the sequence of amino acids ERFFPSNGI (SEQ ID NO:43) at positions 532-540, with numbering relative to SEQ ID NO:5; c) the sequence of amino acids TGATNKTTLENVLMTNEEEIRP (SEQ ID NO:33) at positions 546-567, with numbering relative to SEQ ID NO:5; and d) the sequence of amino acids SSNLQAANTAAQTQVVNN (SEQ ID NO:34) at positions 580-597, with numbering relative to SEQ ID NO:5.

Also provided is a capsid polypeptide, comprising: (i) the sequence of amino acids set forth in SEQ ID NO:6 or a sequence having at least or about 85% sequence identity thereto; (ii) the sequence of amino acids at positions 138-736 of SEQ ID NO:5 or a sequence having at least or about 85% sequence identity thereto; and/or (iii) the sequence of amino acids at positions 204-736 of SEQ ID NO:5 or a sequence having at least or about 85% sequence identity thereto; wherein the capsid polypeptide comprises: a) amino acid residues S263, Q264, S265, S268 and H272, with numbering relative to SEQ ID NO:5; b) amino acid residues T547, G548, T550, N551, K552, T553, T554, L555, E556, N557, L559, M560, N562, R567 and P568, with numbering relative to SEQ ID NO:5; and c) amino acid residues S581, S582, A586, A587, A591, T593, Q594, V595, and N598, with numbering relative to SEQ ID NO:5. In some examples, the capsid polypeptide comprises a) the sequence of amino acids SQSGASNDNH (SEQ ID NO:32) at positions 263-272, with numbering relative to SEQ ID NO:5; b) the sequence of amino acids TGATNKTTLENVLMTNEEEIRP (SEQ ID NO:33) at positions 547-568, with numbering relative to SEQ ID NO:5; and c) the sequence of amino acids SSNLQAANTAAQTQVVNN (SEQ ID NO:34) at positions 581-598, with numbering relative to SEQ ID NO:5.

In a further aspect, provided is a capsid polypeptide, comprising: (i) the sequence of amino acids set forth in SEQ ID NO:7 or a sequence having at least or about 85% sequence identity thereto; (ii) the sequence of amino acids at positions 138-737 of SEQ ID NO:7 or a sequence having at least or about 85% sequence identity thereto; and/or (iii) the sequence of amino acids at positions 204-737 of SEQ ID NO:7 or a sequence having at least or about 85% sequence identity thereto; wherein the capsid polypeptide comprises: a) amino acid residues N263, G264, T265, S266, G267, T270 and T274, with numbering relative to SEQ ID NO:7; b) amino acid residues S453, Q458, G459, Q462, L464, A468, A471, N472, S474 and A475, with numbering relative to SEQ ID NO:7; and c) amino acid residues T548, G549, T551, N552, K553, T554, T555, L556, E557, N558, L560, M561, N563, R568 and P569, with numbering relative to SEQ ID NO:7. In some embodiments, the capsid polypeptide of comprises a) the sequence of amino acids NGTSGGATNDNT (SEQ ID NO:44) at positions 263-274, with numbering relative to SEQ ID NO:7; b) the sequence of amino acids STGGTQGTQQLLFSQAGPANMSA (SEQ ID NO:36) at positions 453-475, with numbering relative to SEQ ID NO:7; and c) the sequence of amino acids TGATNKTTLENVLMTNEEEIRP (SEQ ID NO:33) at positions 548-569, with numbering relative to SEQ ID NO:7.

In another aspect, provided is a capsid polypeptide, comprising: (i) the sequence of amino acids set forth in SEQ ID NO:8 or a sequence having at least or about 85% sequence identity thereto; (ii) the sequence of amino acids at positions 138-737 of SEQ ID NO:8 or a sequence having at least or about 85% sequence identity thereto; and/or (iii) the sequence of amino acids at positions 204-737 of SEQ ID NO:8 or a sequence having at least or about 85% sequence identity thereto; wherein the capsid polypeptide comprises: a) amino acid residues N263, G264, T265, S266, G267, T270 and T274, with numbering relative to SEQ ID NO:7, with numbering relative to SEQ ID NO:8; b) amino acid residues S453, Q458, G459, Q462, L464, A468, A471, N472, S474 and A475, with numbering relative to SEQ ID NO:8; and c) amino acid residues T548, G549, T551, N552, K553, T554, T555, L556, E557, N558, L560, M561, N563, R568 and P569, with numbering relative to SEQ ID NO:8. In some embodiments, the capsid polypeptide comprises a) the sequence of amino acids NGTSGGATNDNT (SEQ ID NO:44) at positions 263-274, with numbering relative to SEQ ID NO:5; b) the sequence of amino acids STGGTQGTQQLLFSQAGPANMSA (SEQ ID NO:36) at positions 453-475, with numbering relative to SEQ ID NO:5; and c) the sequence of amino acids TGATNKTTLENVLMTNEEEIRP (SEQ ID NO:33) at positions 548-569, with numbering relative to SEQ ID NO:5.

Also provided is a capsid polypeptide, comprising: (i) the sequence of amino acids set forth in any one of SEQ ID NOs:9-16, or a sequence having at least or about 95%, 96%, 97%, 98% or 99% sequence identity thereto; (ii) the sequence of amino acids at positions 138-737 of any one of SEQ ID NOs:9-11, 13 and 15, or positions 138-738 of any one of SEQ ID NOs:12, 14 and 16; or a sequence having at least or about 95%, 96%, 97%, 98% or 99% sequence identity thereto; and/or (iii) the sequence of amino acids at positions 204-737 of any one of SEQ ID NOs: 9-11, 13 and 15, or positions 204-738 of any one of SEQ ID NOs: 12, 14 and 16; or a sequence having at least or about 95%, 96%, 97%, 98% or 99% sequence identity thereto.

In a further aspect, provided is a capsid polypeptide, comprising: (i) the sequence of amino acids set forth in any one of SEQ ID NOs:9-16, or a sequence having at least or about 85%, 90%, 95%, 96%, 97%, 98% or 99% sequence identity thereto; (ii) the sequence of amino acids at positions 138-737 of any one of SEQ ID NOs:9-11, 13 and 15, or positions 138-738 of any one of SEQ ID NOs:12, 14 and 16; or a sequence having at least or about 85%, 90%, 95%, 96%, 97%, 98% or 99% sequence identity thereto; and/or (iii) the sequence of amino acids at positions 204-737 of any one of SEQ ID NOs: 9-11, 13 and 15, or positions 204-738 of any one of SEQ ID NOs: 12, 14 and 16; or a sequence having at least or about 85%, 90%, 95%, 96%, 97%, 98% or 99% sequence identity thereto; wherein the capsid polypeptide comprises one or more of: a) amino acid residues T546, G547, T549, N550, K551, T552, T553, L554, E555, N556, L558, M559, N561, R566 and P567, with numbering relative to SEQ ID NO:4; b) amino acid residues S580, S581, A585, A586, A590, T592, Q593, V594, and N597, with numbering relative to SEQ ID NO:4; c) amino acid residues D532, S538 and V540, with numbering relative to SEQ ID NO:4; d) amino acid residues S451, Q456, G457, Q460, L462, A466, A469, N470, S472 and A473, with numbering relative to SEQ ID NO:4; e) amino acid residues L493, S494, G505, A506, V518 and V522, with numbering relative to SEQ ID NO:4; f) the sequence of amino acids TGATNKTTLENVLMTNEEEIRP (SEQ ID NO:33) at positions 546-567, with numbering relative to SEQ ID NO:4; g) the sequence of amino acids SSNLQAANTAAQTQVVNN (SEQ ID NO:34) at positions 580-597, with numbering relative to SEQ ID NO:4; h) the sequence of amino acids DRFFPSSGV (SEQ ID NO:35) at positions 532-540, with numbering relative to SEQ ID NO:4; i) the sequence of amino acids STGGTQGTQQLLFSQAGPANMSA (SEQ ID NO:36) at positions 451-473, with numbering relative to SEQ ID NO:4; and j) the sequence of amino acids LSQNNNSNFAWTGATKYHLNGRNSLVNPGV (SEQ ID NO:37) at positions 493-522, with numbering relative to SEQ ID NO:4.

Also provided are capsid polypeptides comprising: (i) the sequence of amino acids set forth in any one of SEQ ID NOs:47-55, or a sequence having at least or about 97%, 98% or 99% sequence identity thereto; (ii) the sequence of amino acids at positions 138-735 of any one of SEQ ID NOs:47-49, 52, 54 and 55 or positions 138-736 of any one of SEQ ID NOs:50, 51 and 53; or a sequence having at least or about 97%, 98% or 99% sequence identity thereto; and/or (iii) the sequence of amino acids at positions 204-735 of any one of SEQ ID NOs: 47-49, 52, 54 and 55, or positions 204-736 of any one of SEQ ID NOs: 50, 51 and 53; or a sequence having at least or about 97%, 98% or 99% sequence identity thereto.

In another aspect, provided is a capsid polypeptide comprising (i) the sequence of amino acids set forth in SEQ ID NO:47, or a sequence having at least or about 85%, 90%, 95%, 96%, 97%, 98% or 99% sequence identity thereto; (ii) the sequence of amino acids at positions 138-735 of SEQ ID NO:47, or a sequence having at least or about 85%, 90%, 95%, 96%, 97%, 98% or 99% sequence identity thereto; and/or (iii) the sequence of amino acids at positions 204-735 of SEQ ID NOs: 47, or a sequence having at least or about 85%, 90%, 95%, 96%, 97%, 98% or 99% sequence identity thereto; wherein the capsid polypeptide comprises one or more of: a) amino acid residues Q546, S548, A549, D551, A552, H554, D556, M558, L559, and D561, with numbering relative to SEQ ID NO:47; b) the sequence of amino acids QGSANDATHEDVMLTD (SEQ ID NO:65) at positions 546-561, with numbering relative to SEQ ID NO:47; and c) the sequence of amino acids KQGSANDATHEDVMLTDEEEIRP (SEQ ID NO:66) at positions 545-567 with numbering relative to SEQ ID NO:47.

In another aspect, provided is a capsid polypeptide (i) the sequence of amino acids set forth in SEQ ID NO:48, or a sequence having at least or about 85%, 90%, 95%, 96%, 97%, 98% or 99% sequence identity thereto; (ii) the sequence of amino acids at positions 138-735 of SEQ ID NO:48, or a sequence having at least or about 85%, 90%, 95%, 96%, 97%, 98% or 99% sequence identity thereto; and/or (iii) the sequence of amino acids at positions 204-735 of SEQ ID NOs: 48, or a sequence having at least or about 85%, 90%, 95%, 96%, 97%, 98% or 99% sequence identity thereto; wherein the capsid polypeptide comprises one or more of: a) amino acid residues N585, S586, P590, E591, 1592, A593 and N594, with numbering relative to SEQ ID NO:48; b) the sequence of amino acids NSNTAPEIAN (SEQ ID NO:67) at positions 585-594, with numbering relative to SEQ ID NO:48; and c) the sequence of amino acids SSNLQNSNTAPEIANVNN (SEQ ID NO:68) at positions 580-597, with numbering relative to SEQ ID NO:48.

In another aspect, provided is a capsid polypeptide (i) the sequence of amino acids set forth in SEQ ID NO:49, or a sequence having at least or about 85%, 90%, 95%, 96%, 97%, 98% or 99% sequence identity thereto; (ii) the sequence of amino acids at positions 138-735 of SEQ ID NO:49, or a sequence having at least or about 85%, 90%, 95%, 96%, 97%, 98% or 99% sequence identity thereto; and/or (iii) the sequence of amino acids at positions 204-735 of SEQ ID NOs: 49, or a sequence having at least or about 85%, 90%, 95%, 96%, 97%, 98% or 99% sequence identity thereto; wherein the capsid polypeptide comprises one or more of: a) amino acid residues Q546, S548, A549, D551, A552, H554, D556, M558, L559, D561, N585, S586, P590, E591, 1592, A593 and N594, with numbering relative to SEQ ID NO:49; b) the sequence of amino acids QGSANDATHEDVMLTD (SEQ ID NO:65) at positions 546-561, with numbering relative to SEQ ID NO:49; c) the sequence of amino acids KQGSANDATHEDVMLTDEEEIRP (SEQ ID NO:66) at positions 545-567, with numbering relative to SEQ ID NO:49; d) the sequence of amino acids NSNTAPEIAN (SEQ ID NO:67) at positions 585-594, with numbering relative to SEQ ID NO:49; and e) the sequence of amino acids SSNLQNSNTAPEIANVNN (SEQ ID NO:68) at positions 580-597, with numbering relative to SEQ ID NO:49.

In a further aspect, provided is a capsid polypeptide (i) the sequence of amino acids set forth in SEQ ID NO:50, or a sequence having at least or about 85%, 90%, 95%, 96%, 97%, 98% or 99% sequence identity thereto; (ii) the sequence of amino acids at positions 138-736 of SEQ ID NO:50, or a sequence having at least or about 85%, 90%, 95%, 96%, 97%, 98% or 99% sequence identity thereto; and/or (iii) the sequence of amino acids at positions 204-736 of SEQ ID NOs: 50, or a sequence having at least or about 85%, 90%, 95%, 96%, 97%, 98% or 99% sequence identity thereto; wherein the capsid polypeptide comprises one or more of: a) amino acid residues K546, N547, T548, N549, T550, N552, V553, N554, Y555 and M559, with numbering relative to SEQ ID NO:50; b) the sequence of amino acids KNTNTNNVNYENVMM (SEQ ID NO:69) at positions 546-559, with numbering relative to SEQ ID NO:50; and c) the sequence of amino acids KKNTNTNNVNYENVMMTNEEEIRP (SEQ ID NO:70) at positions 545-568, with numbering relative to SEQ ID NO:50.

In another aspect, provided is a capsid polypeptide (i) the sequence of amino acids set forth in SEQ ID NO:51, or a sequence having at least or about 85%, 90%, 95%, 96%, 97%, 98% or 99% sequence identity thereto; (ii) the sequence of amino acids at positions 138-736 of SEQ ID NO:51, or a sequence having at least or about 85%, 90%, 95%, 96%, 97%, 98% or 99% sequence identity thereto; and/or (iii) the sequence of amino acids at positions 204-736 of SEQ ID NOs: 51, or a sequence having at least or about 85%, 90%, 95%, 96%, 97%, 98% or 99% sequence identity thereto; wherein the capsid polypeptide comprises one or more of: a) amino acid residues G447, A451, P453, T457, Q459, N460 and T461, with numbering relative to SEQ ID NO:51; b) the sequence of amino acids GRTQATPGGTTGQNT (SEQ ID NO:71) at positions 447-461, with numbering relative to SEQ ID NO:51; and c) the sequence of amino acids GRTQATPGGTTGQNTLLFSQAGPANMSA (SEQ ID NO:72) at positions 447-474, with numbering relative to SEQ ID NO:51.

In another aspect, provided is a capsid polypeptide (i) the sequence of amino acids set forth in SEQ ID NO:52, or a sequence having at least or about 85%, 90%, 95%, 96%, 97%, 98% or 99% sequence identity thereto; (ii) the sequence of amino acids at positions 138-735 of SEQ ID NO:52, or a sequence having at least or about 85%, 90%, 95%, 96%, 97%, 98% or 99% sequence identity thereto; and/or (iii) the sequence of amino acids at positions 204-735 of SEQ ID NOs: 52, or a sequence having at least or about 85%, 90%, 95%, 96%, 97%, 98% or 99% sequence identity thereto; wherein the capsid polypeptide comprises one or more of: a) amino acid residues K585, E586, P589, 1592, E593 and A594, with numbering relative to SEQ ID NO:52; b) the sequence of amino acids KENTPAQIEA (SEQ ID NO:73) at positions 585-594, with numbering relative to SEQ ID NO:52; and c) the sequence of amino acids SSNLQKENTPAQIEAVNN (SEQ ID NO:74) at positions 580-597, with numbering relative to SEQ ID NO:52.

In another aspect, provided is a capsid polypeptide (i) the sequence of amino acids set forth in SEQ ID NO:53, or a sequence having at least or about 85%, 90%, 95%, 96%, 97%, 98% or 99% sequence identity thereto; (ii) the sequence of amino acids at positions 138-736 of SEQ ID NO:53, or a sequence having at least or about 85%, 90%, 95%, 96%, 97%, 98% or 99% sequence identity thereto; and/or (iii) the sequence of amino acids at positions 204-736 of SEQ ID NOs: 53, or a sequence having at least or about 85%, 90%, 95%, 96%, 97%, 98% or 99% sequence identity thereto; wherein the capsid polypeptide comprises one or more of: a) amino acid residues Q546, S547, S548, S549, G550, A551, V553, N554, G557, L560 and G562, with numbering relative to SEQ ID NO:53; b) the sequence of amino acids QSSSGAKVNLEGVLLTG (SEQ ID NO:75) at positions 546-562, with numbering relative to SEQ ID NO:53; and c) the sequence of amino acids KQSSSGAKVNLEGVLLTGEEEIRP (SEQ ID NO:76) at positions 545-568, with numbering relative to SEQ ID NO:53.

In a further aspect, provided is a capsid polypeptide (i) the sequence of amino acids set forth in SEQ ID NO:54, or a sequence having at least or about 85%, 90%, 95%, 96%, 97%, 98% or 99% sequence identity thereto; (ii) the sequence of amino acids at positions 138-735 of SEQ ID NO:54, or a sequence having at least or about 85%, 90%, 95%, 96%, 97%, 98% or 99% sequence identity thereto; and/or (iii) the sequence of amino acids at positions 204-735 of SEQ ID NOs: 54, or a sequence having at least or about 85%, 90%, 95%, 96%, 97%, 98% or 99% sequence identity thereto; wherein the capsid polypeptide comprises one or more of: a) amino acid residues K448, A451, S453, K456, D457 and N459, with numbering relative to SEQ ID NO:54; b) the sequence of amino acids KTQATSGTKDTN (SEQ ID NO:77) at positions 448-459, with numbering relative to SEQ ID NO:54; and c) the sequence of amino acids KTQATSGTKDTNQLLFSQAGPANMSA (SEQ ID NO:78) at positions 448-473, with numbering relative to SEQ ID NO:54.

In another aspect, provided is a capsid polypeptide (i) the sequence of amino acids set forth in SEQ ID NO:55, or a sequence having at least or about 85%, 90%, 95%, 96%, 97%, 98% or 99% sequence identity thereto; (ii) the sequence of amino acids at positions 138-735 of SEQ ID NO:55, or a sequence having at least or about 85%, 90%, 95%, 96%, 97%, 98% or 99% sequence identity thereto; and/or (iii) the sequence of amino acids at positions 204-735 of SEQ ID NOs: 55, or a sequence having at least or about 85%, 90%, 95%, 96%, 97%, 98% or 99% sequence identity thereto; wherein the capsid polypeptide comprises one or more of: a) amino acid residues T585, G586, P590, E591, L593, 1594 and H597, with numbering relative to SEQ ID NO:55; b) the sequence of amino acids TGNTAPETLIVNH (SEQ ID NO:79) at positions 585-597, with numbering relative to SEQ ID NO:55; and c) the sequence of amino acids SSNLQTGNTAPETLIVNH (SEQ ID NO:80) at positions 580-597, with numbering relative to SEQ ID NO:55.

In some embodiments, the capsid polypeptide comprises: (i) amino acid residues S263, Q264, S265, S268 and H272; the sequence of amino acids SQSGASNDNH (SEQ ID NO:32) at positions 263-272, and/or the sequence of amino acids ISSQSGASNDNH (SEQ ID NO:38) at positions 261-272, with numbering relative to SEQ ID NO:4; (ii) amino acid residues L493, S494, G505, A506, V518 and V522, the sequence of amino acids LSQNNNSNFAWTGATKYHLNGRNSLVNPGV (SEQ ID NO:37) at positions 493-522, and/or the sequence of amino acids RVSTTLSQNNNSNFAWTGATKYHLNGRNSLVNPGV (SEQ ID NO:42) at positions 488-522, with numbering relative to SEQ ID NO:4; and (iii) amino acid residues D532, S538 and V540; the sequence of amino acids DRFFPSSGV (SEQ ID NO:35) at positions 532-540; and/or the sequence of amino acids AMATHKDDEDRFFPSSGV (SEQ ID NO:40) at positions 523-540, with numbering relative to SEQ ID NO:4.

In further embodiments, the capsid polypeptide comprises: (i) amino acid residues S451, Q456, G457, Q460, L462, A466, A469, N470, S472 and A473; the sequence of amino acids STGGTQGTQQLLFSQAGPANMSA (SEQ ID NO:36) at positions 451-473; and/or the sequence of amino acids QSTGGTQGTQQLLFSQAGPANMSA (SEQ ID NO:41) at positions 450-473; with numbering relative to SEQ ID NO:4; and (ii) amino acid residues S580, S581, A585, A586, A590, T592, Q593, V594, and N597; and/or the sequence of amino acids SSNLQAANTAAQTQVVNN (SEQ ID NO:34) at positions 580-597, with numbering relative to SEQ ID NO:4.

In other embodiments, the capsid polypeptide comprises: (i) amino acid residues S451, Q456, G457, Q460, L462, A466, A469, N470, S472 and A473; the sequence of amino acids STGGTQGTQQLLFSQAGPANMSA (SEQ ID NO:36) at positions 451-473; and/or the sequence of amino acids QSTGGTQGTQQLLFSQAGPANMSA (SEQ ID NO:41) at positions 450-473; with numbering relative to SEQ ID NO:4; and (ii) amino acid residues amino acid residues T546, G547, T549, N550, K551, T552, T553, L554, E555, N556, L558, M559, N561, R566 and P567; the sequence of amino acids TGATNKTTLENVLMTNEEEIRP (SEQ ID NO:33) at positions 546-567; and/or the sequence of amino acids KTGATNKTTLENVLMTNEEEIRP (SEQ ID NO:39) at positions 545-567, with numbering relative to SEQ ID NO:4.

In one embodiment, the capsid polypeptide comprises amino acid residues S451, Q456, G457, Q460, L462, A466, A469, N470, S472 and A473; the sequence of amino acids STGGTQGTQQLLFSQAGPANMSA (SEQ ID NO:36) at positions 451-473; and/or the sequence of amino acids QSTGGTQGTQQLLFSQAGPANMSA (SEQ ID NO:41) at positions 450-473; with numbering relative to SEQ ID NO:4.

In other embodiments, the capsid polypeptide comprises: (i) amino acid residues amino acid residues T546, G547, T549, N550, K551, T552, T553, L554, E555, N556, L558, M559, N561, R566 and P567; the sequence of amino acids TGATNKTTLENVLMTNEEEIRP (SEQ ID NO:33) at positions 546-567; and/or the sequence of amino acids KTGATNKTTLENVLMTNEEEIRP (SEQ ID NO:39) at positions 545-567, with numbering relative to SEQ ID NO:4; and (ii) amino acid residues S580, S581, A585, A586, A590, T592, Q593, V594, and N597; and/or the sequence of amino acids SSNLQAANTAAQTQVVNN (SEQ ID NO:34) at positions 580-597, with numbering relative to SEQ ID NO:4.

Also provided is an AAV vector, comprising a capsid polypeptide of the present disclosure. In some examples, the vector further comprises a heterologous coding sequence, such as a heterologous coding sequence that encodes a peptide, polypeptide or polynucleotide (e.g. a therapeutic peptide, polypeptide or polynucleotide).

In another aspect, provided is a isolated nucleic acid molecule encoding a capsid polypeptide of the present disclosure. In a further aspect, provided is a vector comprising the aforementioned nucleic acid molecule. In some examples, the vector is selected from among a plasmid, cosmid, phage and transposon.

Also provided is a host cell, comprising an AAV vector, nucleic acid molecule or vector of the present disclosure.

In a further aspect, provided is a method for introducing a heterologous coding sequence into a host cell, comprising contacting a host cell with an AAV vector of the present disclosure. In some examples, the host cell is a hepatocyte. In one embodiment, contacting a host cell with the AAV vector comprises administering the AAV vector to a subject. In another embodiment, the method is in vitro or ex vivo.

In another aspect, provided is a method for producing an AAV vector, comprising culturing a host cell comprising a nucleic acid molecule encoding a capsid polypeptide of the present disclosure, an AAV rep gene, a heterologous coding sequence flanked by AAV inverted terminal repeats, and helper functions for generating a productive AAV infection, under conditions suitable to facilitate assembly of an AAV vector comprising a capsid comprising a capsid polypeptide of the present disclosure, wherein the capsid encapsidates the heterologous coding sequence. In some examples, the host cell is a hepatocyte.

Also provided is a method for producing a modified AAV vector that exhibits enhanced transgene expression in a human hepatocyte hen the vector comprises a transgene, comprising: a) identifying a reference capsid polypeptide for transducing human hepatocytes in vivo; b) modifying the sequence of the reference capsid polypeptide at one or more of positions 263, 264, 265, 266, 267, 270, 274, 453, 458, 459, 462, 464, 468, 471, 472, 474, 475, 548, 549, 551, 552, 553, 554, 555, 556, 557, 558, 560, 561, 563 and 568, with numbering relative to SEQ ID NO:7, to thereby produce a modified capsid polypeptide that comprises: i) amino acid residues N263, G264, T265, S266, G267, T270 and T274, with numbering relative to SEQ ID NO:7; ii) amino acid residues S453, Q458, G459, Q462, L464, A468, A471, N472, S474 and A475, with numbering relative to SEQ ID NO:7; and (iii) amino acid residues T548, G549, T551, N552, K553, T554, T555, L556, E557, N558, L560, M561, N563, R568 and P569, with numbering relative to SEQ ID NO:7; and c) vectorising the modified capsid polypeptide to thereby produce a modified AAV vector.

Also provided is a method for producing a modified AAV vector that exhibits enhanced transgene expression in a human hepatocyte when the vector comprises a transgene, comprising: a) identifying a reference capsid polypeptide for transducing human hepatocytes in vivo; b) modifying the sequence of the reference capsid polypeptide at one or more of positions 263-274, 453-475 and 548-569 with numbering relative to SEQ ID NO:7, to thereby produce a modified capsid polypeptide that comprises: i) the sequence of amino acids NGTSGGATNDNT (SEQ ID NO:44 at positions 263-274, with numbering relative to SEQ ID NO:7; ii) the sequence of amino acids STGGTQGTQQLLFSQAGPANMSA (SEQ ID NO:36) at positions 453-475, with numbering relative to SEQ ID NO:7; and iii) the sequence of amino acids TGATNKTTLENVLMTNEEEIRP (SEQ ID NO:33) at positions 548-569, with numbering relative to SEQ ID NO:7; and c) vectorising the modified capsid polypeptide to thereby produce a modified AAV vector.

In some examples, the reference capsid polypeptide comprises at least or about 85%, 86%, 87%, 88%, 89%, 90%, 91%, 92%, 93%, 94%, 95%, 96%, 97%, 98%, or 99% sequence identity to the sequence set forth in SEQ ID NO:7. In one embodiment, the modified AAV vector comprises a transgene. In further embodiments, the method comprises assessing the transgene expression of the modified AAV vector in an in vivo system (e. g. one that comprises a small animal, such as a mouse) with a chimeric liver comprising human hepatocytes, e.g. a humanised FRG mouse.

### BRIEF DESCRIPTION OF THE DRAWINGS

Embodiments of the disclosure are described herein, by way of non-limiting example only, with reference to the following drawings.

FIG. 1 is a representation of the in vivo performance of AAVC11.12-Venus, AAV8-Cerulean, AAV8-SF-Venus, AAV7-Cerulean and AAV7-SF-Venus in humanised FRG mice. Vectors were injected into humanised FRG mice with low or high RI and entry into mouse and human hepatocytes in the liver was analysed by immunofluorescence. (A) Percentage of Venus positive and Cerulean positive human and murine hepatocytes on the same humanised FRG mouse (RI=0.6%) injected with AAVC11.12-Venus (hatched bars) and AAV8-Cerulean (filled bars) as analysed with FACS. (B) Percentage of Venus positive and Cerulean positive human and murine hepatocytes on the same humanised FRG mouse (RI=1.5%) injected with AAV8-SF-Venus (hatched bars) and AAV8-Cerulean (filled bars) as analysed with FACS. (C) Percentage of Venus positive and Cerulean positive human and murine hepatocytes on the same humanised FRG mouse (RI=4.8%) injected with AAV7-SF-Venus (hatched bars) and AAV7-Cerulean (filled bars) as analysed with FACS. (D) Percentage of Venus positive and Cerulean positive human and murine hepatocytes on the same humanised FRG mouse (RI=84.4%) injected with AAVC11.12-Venus (hatched bars) and AAV8-Cerulean (filled bars) as analysed with FACS. (E) Percentage of Venus positive and Cerulean positive human and murine hepatocytes on the same humanised FRG mouse (RI=82.7%) injected with AAV8-SF-Venus (hatched bars) and AAV8-Cerulean (filled bars) as analysed with FACS. (F) Percentage of Venus positive and Cerulean positive human and murine hepatocytes on the same humanised FRG mouse (RI=85.7%) injected with AAV7-SF-Venus (hatched bars) and AAV7-Cerulean (filled bars) as analysed with FACS.

FIG. 2 is a schematic representation of AAV variable regions swapped into the AAV8 capsid scaffold.

FIG. 3 is an alignment of the sequences of the AAV8 and AAVC11.12 capsid polypeptides. Variable region (VR)-I, VR-IV, VR-V, VR-VI, VR-VII and VR-III are shown, with residues making up those regions bolded and in italics in the AAV8 polypeptide. Residues from AAVC11.12 that were used to replace the corresponding residue in AAV8 are underlined, and the region spanning the first and last replacement for each variable region is shaded in grey.

FIG. 4 is a representation of the in vivo performance of AAVC11.12, AAV8, Swap6 and Swap16-Swap23 in high-RI (replacement index (RI) of 89%) humanised FRG mice (N=2). (A) The percentage of NGS reads mapped to each AAV capsid (sum of n=4 barcodes/capsid) in human hepatocytes at the DNA (vector uptake, physical transduction) and cDNA (expression, functional transduction) level, normalized to the pre-injection mix, is shown. (B) Expression indexes (raw cDNA read percentage/raw DNA read percentage) of the swapped variants.

FIG. 5 is a representation of the in vivo performance of AAV8-Cerulean, AAV8-EE-Venus, AAV7-Cerulean and AAV7-EE-Venus in humanised FRG mice (N=2). Vectors were injected into humanised FRG mice and entry into mouse and human hepatocytes in the liver was analysed by immunofluorescence. The percentage of Venus positive and Cerulean positive human and murine hepatocytes was determined. (A) Percentage of murine and human hepatocytes positive for AAV8-Cerulean (filled bars) or AAV8-EE-Venus (hatched bars). (B) Percentage of murine and human hepatocytes positive for AAV7-Cerulean (filled bars) or AAV7-EE-Venus (hatched bars).

FIG. 6 is an alignment of various AAV capsid sequences.

FIG. 7 is an alignment of various AAV capsid sequences.

FIG. 8 is a representation of the in vivo performance (DNA uptake and cDNA expression in human hepatocytes) of AAV capsid variants generated from the C11.12 backbone as described in Example 4, in high-RI FRG mice that were not passively immunized (A) or that were passively immunized with 5 mg IVIg (B).

### DETAILED DESCRIPTION

Unless defined otherwise, all technical and scientific terms used herein have the same meaning as is commonly understood by one of skill in the art to which the disclosure belongs. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

As used herein, the singular forms “a”, “an” and “the” also include plural aspects (i.e. at least one or more than one) unless the context clearly dictates otherwise. Thus, for example, reference to “a polypeptide” includes a single polypeptide, as well as two or more polypeptides.

In the context of this specification, the term “about,” is understood to refer to a range of numbers that a person of skill in the art would consider equivalent to the recited value in the context of achieving the same function or result.

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, a “vector” includes reference to both polynucleotide vectors and viral vectors, each of which are capable of delivering a transgene contained within the vector into a host cell. Vectors can be episomal, i.e., do not integrate into the genome of a host cell, or can integrate into the host cell genome. The vectors may also be replication competent or replication deficient. Exemplary polynucleotide vectors include, but are not limited to, plasmids, cosmids and transposons. Exemplary viral vectors include, for example, AAV, lentiviral, retroviral, adenoviral, herpes viral and hepatitis viral vectors.

As used herein, “adeno-associated viral vector” or “AAV vector” refers to a vector in which the capsid is derived from an adeno-associated virus, including without limitation, AAV1, AAV2, AAV3, AAV4, AAV5, AAV6, AAV7, AAV8, AAV9, AAV10, AAV11, AAV12 or AAV13, AAV from other clades or isolates, or is derived from synthetic, bioengineered or modified AAV capsid proteins, including chimeric capsid proteins. In particular embodiments, the AAV vector has a capsid comprising a capsid polypeptide of the present disclosure. When referring to AAV vectors, both the source of the genome and the source of the capsid can be identified, where the source of the genome is the first number designated and the source of the capsid is the second number designated. Thus, for example, a vector in which both the capsid and genome are derived from AAV2 is more accurately referred to as AAV2/2. A vector with an AAV6-derived capsid and an AAV2-derived genome is most accurately referred to as AAV2/6. A vector with the bioengineered DJ capsid and an AAV2-derived genome is most accurately referred to as AAV2/DJ. For simplicity, and because most vectors use an AAV2-derived genome, it is understood that reference to an AAV6 vector generally refers to an AAV2/6 vector, reference to an AAV2 vector generally refers to an AAV2/2 vector, etc. An AAV vector may also be referred to herein as “recombinant AAV”, “rAAV”, “recombinant AAV virion”, “rAAV virion”, “AAV variant”, “recombinant AAV variant”, and “rAAV variant” terms which are used interchangeably and refer to a replication-defective virus that includes an AAV capsid shell encapsidating an AAV genome. The AAV vector genome (also referred to as vector genome, recombinant AAV genome or rAAV genome) comprises a transgene flanked on both sides by functional AAV ITRs. Typically, one or more of the wild-type AAV genes have been deleted from the genome in whole or part, preferably the rep and/or cap genes. Functional ITR sequences are necessary for the rescue, replication and packaging of the vector genome into the rAAV virion.

The term “ITR” refers to an inverted terminal repeat at either end of the AAV genome. This sequence can form hairpin structures and is involved in AAV DNA replication and rescue, or excision, from prokaryotic plasmids. ITRs for use in the present disclosure need not be the wild-type nucleotide sequences, and may be altered, e.g., by the insertion, deletion or substitution of nucleotides, as long as the sequences provide for functional rescue, replication and packaging of rAAV.

As used herein, “functional” with reference to a capsid polypeptide means that the polypeptide can self-assemble or assemble with different capsid polypeptides to produce the proteinaceous shell (capsid) of an AAV virion. It is to be understood that not all capsid polypeptides in a given host cell assemble into AAV capsids. Preferably, at least 25%, at least 50%, at least 75%, at least 85%, at least 90%, at least 95% of all AAV capsid polypeptide molecules assemble into AAV capsids. Suitable assays for measuring this biological activity are described e.g. in Smith-Arica and Bartlett (2001), Curr Cardiol Rep 3(1): 43-49.

“AAV helper functions” or “helper functions” refer to functions that allow AAV to be replicated and packaged by a host cell. AAV helper functions can be provided in any of a number of forms, including, but not limited to, as a helper virus or as helper virus genes which aid in AAV replication and packaging. Helper virus genes include, but are not limited to, adenoviral helper genes such as E1A, E1B, E2A, E4 and VA. Helper viruses include, but are not limited to, adenoviruses, herpesviruses, poxviruses such as vaccinia, and baculovirus. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Baculoviruses available from depositories include Autographa californica nuclear polyhedrosis virus.

As used herein, the term “transduction” refers to entry of AAV vector into one or more particular cell types and transferal of the DNA contained within the AAV vector into the cell. Transduction can be assessed by measuring the amount of AAV DNA or RNA expressed from the AAV DNA in a cell or population of cells, and/or by assessing the number of cells in a population that contain AAV DNA or RNA expressed from the DNA. Where the presence or amount of RNA is assessed, the type of transduction assessed is referred to herein as “functional transduction”, i.e. the ability of the AAV to transfer DNA to the cell and have that DNA expressed. The term “transduction efficiency” and grammatical variations thereof refers to the ability of an AAV vector to transduce host cells, and more particularly the efficiency with which an AAV vector transduces host cells. In particular embodiment, the transduction efficiency is in vivo transduction efficiency, and refers to the ability of an AAV vector to transduce host cells in vivo following administration of the vector to the subject. Transduction efficiency can be assessed in a number of ways known to those in the art, including assessing the number of host cells transduced following exposure to, or administration of, a given number of vector particles (e.g. as assessed by expression of a reporter gene from the vector genome, such as GFP or eGFP, using microscopy or flow cytometry techniques); the amount of vector DNA (e.g. number of vector genomes) in a population of host cells following exposure to a given number of vector particles; the amount of vector RNA in population of host cells following exposure to a given number of vector particles; and the level of protein expression from a reporter gene (e.g. GFP or eGFP) in the vector genome in a population of host cells following exposure to, or administration of, a given number of vector particles. The population of host cells can represent a particular number of host cells, a volume or weight of tissue, or an entire organ (e.g. liver). In vivo transduction efficiency can reflect the ability of an AAV vector to access host cells, such as hepatocytes in the liver; the ability of an AAV vector to enter host cells; and/or expression of a heterologous coding sequence contained in the vector genome upon host cell entry.

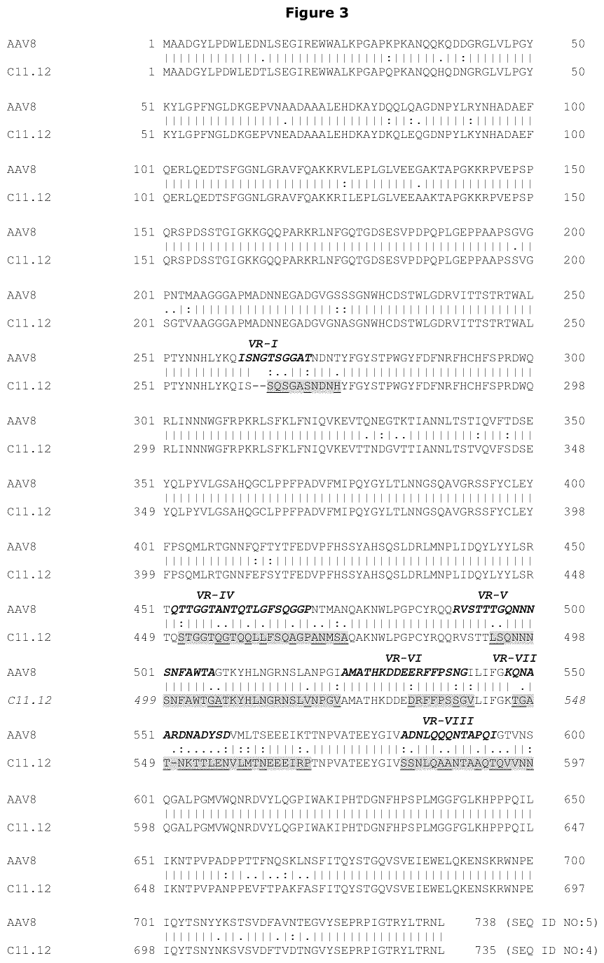

As used herein, “corresponding nucleotides”, “corresponding amino acid residues” or “corresponding positions” refer to nucleotides, amino acids or positions that occur at aligned loci. The sequences of related or variant polynucleotides or polypeptides are aligned by any method known to those of skill in the art. Such methods typically maximize matches (e.g. identical nucleotides or amino acids at positions), and include methods such as using manual alignments and by using the numerous alignment programs available (for example, BLASTN, BLASTP, ClustlW, ClustlW2, EMBOSS, LALIGN, Kalign, etc) and others known to those of skill in the art. By aligning the sequences of polynucleotides, one skilled in the art can identify corresponding nucleotides. For example, by aligning two AAV capsid polypeptides (e.g. as shown in FIG. 3), one of skill in the art can identify regions or amino acids residues within one AAV polypeptide that correspond to various regions or residues in the other AAV polypeptide. For example, the leucine at position 738 of the AAV8 capsid polypeptide set forth in SEQ ID NO:5 is the corresponding amino acid of, or corresponds to, the leucine at position 735 of the AAVC11.12 capsid polypeptide set forth in SEQ ID NO:4 (see the alignment of the capsid polypeptides of AAV8 and AAVC11.12 FIG. 3). In another example, and with reference to the same alignment, position 263 of the AAVC11.12 capsid polypeptide aligns with, or corresponds to, position 265 of the AAV8 capsid polypeptide, and the serine at position 263 of the AAVC11.12 capsid polypeptide corresponds to, or is the corresponding amino acid of, the threonine at position 265 of the AAV8 capsid polypeptide. Thus, when amino acid residues or positions are referred to herein with respect to a particular capsid polypeptide, it is understood that, where appropriate, the reference is also to the corresponding amino acid residue or position in another capsid polypeptide. For example, reference to a capsid polypeptide comprising “S263 with numbering relative to SEQ ID NO:4” encompasses not only the AAVC11.12 capsid polypeptide set forth in SEQ ID NO:13 having a serine at position 263, but also other capsid polypeptides having a serine at the position that corresponds to position 263 of SEQ ID NO:4.

A “heterologous coding sequence” as used herein refers to nucleic acid sequence present in a polynucleotide, vector, or host cell that is not naturally found in the polynucleotide, vector, or host cell or is not naturally found at the position that it is at in the polynucleotide, vector, or host cell, i.e. is non-native. A “heterologous coding sequence” can encode a peptide or polypeptide, or a polynucleotide that itself has a function or activity, such as an antisense or inhibitory oligonucleotide, including antisense DNA and RNA (e.g. miRNA, siRNA, and shRNA). In some examples, the heterologous coding sequence is a stretch of nucleic acids that is essentially homologous to a stretch of nucleic acids in the genomic DNA of an animal, such that when the heterologous coding sequence is introduced into a cell of the animal, homologous recombination between the heterologous sequence and the genomic DNA can occur. In one example, the heterologous coding sequence is a functional copy of a gene for introduction into a cell that has a defective/mutated copy.

As used herein, the term “operably-linked” with reference to a promoter and a coding sequence means that the transcription of the coding sequence is under the control of, or driven by, the promoter.

The term “host cell” refers to a cell, such as a mammalian cell, that has introduced into it the exogenous DNA, such as a vector or other polynucleotide. The term includes the progeny of the original cell into which the exogenous DNA has been introduced. Thus, a “host cell” as used herein generally refers to a cell that has been transfected or transduced with exogenous DNA.

As used herein, “isolated” with reference to a polynucleotide or polypeptide means that the polynucleotide or polypeptide is substantially free of cellular material or other contaminating proteins from the cells from which the polynucleotide or polypeptide is derived, or substantially free from chemical precursors or other chemicals when chemically synthesized.

The term “subject” as used herein refers to an animal, in particular a mammal and more particularly a primate including a lower primate and even more particularly, a human who can benefit from the present invention. A subject, regardless of whether a human or non-human animal or embryo, may be referred to as an individual, subject, animal, patient, host or recipient. The present disclosure has both human and veterinary applications. For convenience, an “animal” specifically includes livestock animals such as cattle, horses, sheep, pigs, camelids, goats and donkeys, as well as domestic animals, such as dogs and cats. With respect to horses, these include horses used in the racing industry as well as those used recreationally or in the livestock industry. Examples of laboratory test animals include mice, rats, rabbits, guinea pigs and hamsters. Rabbits and rodent animals, such as rats and mice, provide a convenient test system or animal model as do primates and lower primates. In some embodiments, the subject is human.

As used herein, the term “conservative sequence modifications” or “conservative substitution” refers to amino acid modifications that do not significantly affect or alter the characteristics of a vector containing the amino acid sequence. Such conservative modifications include amino acid substitutions, additions and deletions. Modifications can be introduced into a vector that are compatible with various embodiments by standard techniques known in the art, such as site-directed mutagenesis and PCR-mediated mutagenesis. Conservative amino acid substitutions are ones in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a capsid can be replaced with other amino acid residues from the same side chain family and the altered capsid can be tested for tropism and/or the ability to deliver a payload using the functional assays described herein.

It will be appreciated that the above described terms and associated definitions are used for the purpose of explanation only and are not intended to be limiting.

TABLE 1
Brief Description of the Sequences

| SEQ | |
|---|---|
| ID | |
| NO. | Description/Sequence |
| 1 | Prototypic AAV2 capsid polypeptide |
| 2 | Prototypic AAV7 capsid polypeptide |
| 3 | Prototypic AAV8 capsid polypeptide |
| 4 | AAVC11.12 capsid polypeptide |
| 5 | AAV8-SF capsid polypeptide |
| 6 | AAV7-SF capsid polypeptide |
| 7 | AAV8-EE capsid polypeptide |
| 8 | AAV7-EE capsid polypeptide |
| 9 | AAV8 Swap16 capsid polypeptide |
| 10 | AAV8 Swap17 capsid polypeptide |
| 11 | AAV8 Swap18 capsid polypeptide |
| 12 | AAV8 Swap19 capsid polypeptide |
| 13 | AAV8 Swap20 capsid polypeptide |
| 14 | AAV8 Swap21 capsid polypeptide |
| 15 | AAV8 Swap22 capsid polypeptide |
| 16 | AAV8 Swap23 capsid polypeptide |
| 17 | AAV7 capsid polynucleotide |
| 18 | AAV8 capsid polynucleotide |
| 19 | AAVC11.12 capsid polynucleotide |
| 20 | AAV8-SF capsid polynucleotide |
| 21 | AAV7-SF capsid polynucleotide |
| 22 | AAV8-EE capsid polynucleotide |
| 23 | AAV7-EE capsid polynucleotide |
| 24 | AAV8 Swap16 capsid polynucleotide |
| 25 | AAV8 Swap17 capsid polynucleotide |
| 26 | AAV8 Swap18 capsid polynucleotide |
| 27 | AAV8 Swap19 capsid polynucleotide |
| 28 | AAV8 Swap20 capsid polynucleotide |
| 29 | AAV8 Swap21 capsid polynucleotide |
| 30 | AAV8 Swap22 capsid polynucleotide |
| 31 | AAV8 Swap23 capsid polynucleotide |
| 32 | SQSGASNDNH |
| 33 | TGATNKTTLENVLMTNEEEIRP |
| 34 | SSNLQAANTAAQTQVVNN |
| 35 | DRFFPSSGV |
| 36 | STGGTQGTQQLLFSQAGPANMSA |
| 37 | LSQNNNSNFAWTGATKYHLNGRNSLVNPGV |
| 38 | ISSQSGASNDNH |
| 39 | KTGATNKTTLENVLMTNEEEIRP |
| 40 | AMATHKDDEDRFFPSSGV |
| 41 | QSTGGTQGTQQLLFSQAGPANMSA |
| 42 | RVSTTLSQNNNSNFAWTGATKYHLNGRNSLVNPGV |
| 43 | ERFFPSNGI |
| 44 | NGTSGGATNDNT |
| 45 | AMATHKDDEERFFPSNGI |
| 46 | ISNGTSGGATNDNT |
| 47 | AAV C11.12.253R4.1 capsid polypeptide |
| 48 | AAV C11.12.253R4.3 capsid polypeptide |
| 49 | AAV C11.12.253R4.6 capsid polypeptide |
| 50 | AAV C11.12.253R4.9 capsid polypeptide |
| 51 | AAV C11.12.269R4.1 capsid polypeptide |
| 52 | AAV C11.12.269R4.3 capsid polypeptide |
| 53 | AAV C11.12.269R4.5 capsid polypeptide |
| 54 | AAV C11.12.269R4.6 capsid polypeptide |
| 55 | AAV C11.12.269R4.20 capsid polypeptide |
| 56 | AAV C11.12.253R4.1 capsid polynucleotide |
| 57 | AAV C11.12.253R4.3 capsid polynucleotide |
| 58 | AAV C11.12.253R4.6 capsid polynucleotide |
| 59 | AAV C11.12.253R4.9 capsid polynucleotide |
| 60 | AAV C11.12.269R4.1 capsid polynucleotide |
| 61 | AAV C11.12.269R4.3 capsid polynucleotide |
| 62 | AAV C11.12.269R4.5 capsid polynucleotide |
| 63 | AAV C11.12.269R4.6 capsid polynucleotide |
| 64 | AAV C11.12.269R4.20 capsid polynucleotide |
| 65 | QGSANDATHEDVMLTD |
| 66 | KQGSANDATHEDVMLTDEEEIRP |
| 67 | NSNTAPEIAN |
| 68 | SSNLQNSNTAPEIANVNN |
| 69 | KNTNTNNVNYENVMM |
| 70 | KKNTNTNNVNYENVMMTNEEEIRP |
| 71 | GRTQATPGGTTGQNT |
| 72 | GRTQATPGGTTGQNTLLFSQAGPANMSA |
| 73 | KENTPAQIEA |
| 74 | SSNLQKENTPAQIEAVNNQ |
| 75 | QSSSGAKVNLEGVLLTG |
| 76 | KQSSSGAKVNLEGVLLTGEEEIRP |
| 77 | KTQATSGTKDTN |
| 78 | KTQATSGTKDTNQLLFSQAGPANMSA |
| 79 | TGNTAPETLIVNH |
| 80 | SSNLQTGNTAPETLIVNH |

### Capsid Polypeptides

The present disclosure is predicated in part on the identification of novel AAV capsid polypeptides. Typically, the capsid polypeptides, when present in the capsid of an AAV vector, facilitate efficient transduction of human cells (such as human hepatocytes). The in vivo transduction of cells by AAV vectors having a capsid comprising a capsid polypeptide of the present disclosure is generally increased or enhanced compared to AAV vectors comprising a reference AAV capsid polypeptide (e.g. the prototypic AAV2, AAV7 or AAV8 capsids set forth in SEQ ID NO: 1, 2 or 3). Transduction or transduction efficiency of AAV vectors can be increased by at least or about 10%, 20%, 30%, 40%, 50%, 60%, 70%, 80%, 90%, 100%, 200%, 300%, 400%, 500%, 600%, 700%, 800%, 900%, 1000% or more, e.g. an AAV vector comprising a capsid polypeptide of the present disclosure can be at least or about 1.2×, 1.5×, 2×, 3×, 4×, 5×, 6×, 7×, 8×, 9×, 10×, 11×, 12×, 13×, 14×, 15×, 16×, 17×, 18×, 19×, 20×, 30×, 40×, 50×, 60×, 70×, 80×, 90×, 100× or more efficient at transducing cells in vivo compared to a reference AAV capsid polypeptide (e.g. one set forth in SEQ ID NO:1, 2 or 3). In particular examples, the increased transduction or transduction efficiency is observed in human liver tissue or human hepatocytes.

The capsid polypeptides of the present disclosure are therefore particularly useful in preparing AAV vectors, and in particular AAV vectors for gene therapy uses. In exemplary embodiments, the capsid polypeptides of the present disclosure are particularly useful in preparing AAV vectors that transduce hepatocytes, and in particular, human hepatocytes, and are thus useful for gene therapy applications targeting the liver.

Provided herein are polypeptides, including isolated polypeptides, comprising all or a portion of an AAV capsid polypeptide set forth in any one of SEQ ID NOs: 5-16, including all or a portion of the VP1 protein (comprising amino acid residues corresponding to those at positions 1-735 of SEQ ID NO:1), VP2 protein (comprising amino acid residues corresponding to those at positions 138-735 of SEQ ID NO:3) and/or the VP3 protein (comprising amino acid residues corresponding to those at positions 203-735 of SEQ ID NO:3), and variants thereof, including variants comprising at least or about 90%, 91%, 92%, 93%, 94%, 95%, 96%, 97%, 98%, or 99% sequence identity to the VP1, VP2 or VP3 proteins described herein.

In some examples, the capsid polypeptides comprise all or a portion of one or more variable regions having a sequence that is the same as the sequence of the corresponding variable region present in the AAVC11.12 polypeptide (SEQ ID NO:4). The variable regions of AAV capsid polypeptides have been described (see e.g. Drouin and Agbandje-McKenna, 2013, Future Virol. 8(12): 1183-1199) and include VR-I, spanning positions 260-267; VR-II, spanning positions 326-330; VR-III, spanning positions 380-384; VR-IV, spanning positions 449-467; VR-V, spanning positions 487-504; VR-VI, spanning positions 522-538; VR-VII, spanning positions 544-557; VR-VIII, spanning positions 580-592; and VR-IX, spanning positions 703-711 with numbering relative to AAV2. The AAVC11.12 polypeptide, which was generated from a DNA shuffled library, contains a VR-I of AAV2 origin, VR-IV and VR-V of AAV10 origin, and VR-VI, VR-VII, and VR-VIII of AAV7 origin (when using the VR regions as defined above and in Drouin and Agbandje-McKenna, 2013, the VR-I spans positions 261-268; the VR-IV spans positions 450-468; the VR-V spans positions 488-505; the VR-VI spans positions 523-539; the VR-VII spans positions 545-557; and the VR-VIII spans positions 580-592 of the AAVC11.12 polypeptide set forth in SEQ ID NO:4). Thus, in some examples, the capsid polypeptides of the present disclosure comprise all or a portion of one or more of the VR-I, VR-IV, VR-V, VR-VI, VR-VII and VR-VIII of the AAVC11.12 polypeptide, as described further below.

Capsid polypeptides of the disclosure include those comprising all or a portion of the VP1 protein set forth in SEQ ID NO:5 (also referred to as AAV8-SF) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:5 or comprising a sequence having at least or about 85%, 86%, 87%, 88%, 89%, 90%, 91%, 92%, 93%, 940%, 95%, 96%, 97%, 98%, or 99% sequence identity to the VP2 protein set forth as amino acids 138-735 of SEQ ID NO:5 or a functional fragment thereof; and capsid polypeptides comprising all or a portion of the VP3 protein set forth as amino acids 204-735 of SEQ ID NO:5 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 204-735 of SEQ ID NO:5 or a functional fragment thereof. In particular embodiments, the capsid polypeptide comprises the VR-I, VR-VII and VR-VIII from AAVC11.12 (i.e. the VR-I from AAV2, and the VR-VII and VR-VIII from AAV7) and the VR-VI from AAV8. In particular examples, the capsid polypeptide comprises amino acid residues S263, Q264, S265, S268, H272, E532, N538, 1540, T546, G547, T549, N550, K551, T552, T553, L554, E555, N556, L558, M559, N561, R566, P567, S580, S581, A585, A586, A590, T592, Q593, V594, and N597, with numbering relative to SEQ ID NO:5. In one example, the capsid polypeptide comprises the sequence of amino acids SQSGASNDNH (SEQ ID NO:32) at positions 263-272, the sequence of amino acids ERFFPSNGI (SEQ ID NO:43) at positions 532-540, the sequence of amino acids TGATNKTTLENVLMTNEEEIRP (SEQ ID NO:33) at positions 546-567, and the sequence of amino acids SSNLQAANTAAQTQVVNN (SEQ ID NO:34) at positions 580-597, with numbering relative to SEQ ID NO:5; or sequences having 1, 2, 3, 4 or 5 conservative amino acid substitutions of amino acid residues within the aforementioned sequences. In another example, the capsid polypeptide comprises the sequence of amino acids ISSQSGASNDNH (SEQ ID NO:38 at positions 261-272, the sequence of amino acids AMATHKDDEERFFPSNGI (SEQ ID NO:45) at positions 523-540, the sequence of amino acids KTGATNKTTLENVLMTNEEEIRP (SEQ ID NO:39) at positions 545-567, and the sequence of amino acids SSNLQAANTAAQTQVVNN (SEQ ID NO: 34) at positions 580-597, with numbering relative to SEQ ID NO:5, or sequences having 1, 2, 3, 4 or 5 conservative amino acid substitutions of amino acid residues within the aforementioned sequences.

Capsid polypeptides of the disclosure also include those comprising all or a portion of the VP1 protein set forth in SEQ ID NO:6 (also referred to as AAV7-SF) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO:6 or comprising a sequence having at least or about 85%, 86%, 87%, 88%, 89%, 90%, 91%, 92%, 93%, 940%, 95%, 96%, 97%, 98%, or 99% sequence identity to the VP2 protein set forth as amino acids 138-735 of SEQ ID NO:6 or a functional fragment thereof; and capsid polypeptides comprising all or a portion of the VP3 protein set forth as amino acids 204-735 of SEQ ID NO:6 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 204-735 of SEQ ID NO:6 or a functional fragment thereof. In particular embodiments, the capsid polypeptide comprises the VR-I, VR-VII and VR-VIII from AAVC11.12 (i.e. the VR-I from AAV2, and the VR-VII and VR-VIII from AAV7). In particular examples, the capsid polypeptide comprises amino acid residues S263, Q264, S265, S268, H272, T547, G548, T550, N551, K552, T553, T554, L555, E556, N557, L559, M560, N562, R567, P568, S581, S582, A586, A587, A591, T593, Q594, V595, and N598, with numbering relative to SEQ ID NO:6. In one example, the capsid polypeptide comprises the sequence of amino acids SQSGASNDNH (SEQ ID NO:32) at positions 263-272, the sequence of amino acids TGATNKTTLENVLMTNEEEIRP (SEQ ID NO:33) at positions 547-568, and the sequence of amino acids SSNLQAANTAAQTQVVNN (SEQ ID NO:34) at positions 581-598, with numbering relative to SEQ ID NO:6; or sequences having 1, 2, 3, 4 or 5 conservative amino acid substitutions of amino acid residues within the aforementioned sequences. In another example, the capsid polypeptide comprises the sequence of amino acids ISSQSGASNDNH (SEQ ID NO:38) at positions 261-272, the sequence of amino acids KTGATNKTTLENVLMTNEEEIRP (SEQ ID NO:39) at positions 546-568, and the sequence of amino acids SSNLQAANTAAQTQVVNN (SEQ ID NO:34) at positions 581-598, with numbering relative to SEQ ID NO: 6; or sequences having 1, 2, 3, 4 or 5 conservative amino acid substitutions of amino acid residues within the aforementioned sequences.

Capsid polypeptides of the disclosure also include those comprising all or a portion of the VP1 protein set forth in SEQ ID NO:7 (also referred to as AAV8-EE) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-737 of SEQ ID NO:7 or comprising a sequence having at least or about 85%, 86%, 87%, 88%, 89%, 90%, 91%, 92%, 93%, 940%, 95%, 96%, 97%, 98%, or 99% sequence identity to the VP2 protein set forth as amino acids 138-737 of SEQ ID NO:7 or a functional fragment thereof; and capsid polypeptides comprising all or a portion of the VP3 protein set forth as amino acids 204-737 of SEQ ID NO:7 or comprising a sequence having at least or about 85%, 86%, 87%, 88%, 89%, 90%, 91%, 92%, 93%, 940%, 95%, 96%, 97%, 98%, or 99% sequence identity to the VP3 protein set forth as amino acids 204-737 of SEQ ID NO:7 or a functional fragment thereof. In particular embodiments, the capsid polypeptide comprises the VR-I from AAV8 and the VR-IV and VR-VII from AAVC11.12 (i.e. the VR-IV from AAV10, and the VR-VII from AAV7). In particular examples, the capsid polypeptide comprises amino acid residues N263, G264, T265, S266, G267, T270, T274, S453, Q458, G459, Q462, L464, A468, A471, N472, S474, A475, T548, G549, T551, N552, K553, T554, T555, L556, E557, N558, L560, M561, N563, R568 and P569, with numbering relative to SEQ ID NO:7. In one example, the capsid polypeptide comprises the sequence of amino acids NGTSGGATNDNT (SEQ ID NO:44) at positions 263-274, the sequence of amino acids STGGTQGTQQLLFSQAGPANMSA (SEQ ID NO: 36) at positions 453-475, and the sequence of amino acids TGATNKTTLENVLMTNEEEIRP (SEQ ID NO:33) at positions 548-569, with numbering relative to SEQ ID NO:7; or sequences having 1, 2, 3, 4 or 5 conservative amino acid substitutions within the aforementioned sequences. In a further example, the capsid polypeptide comprises the sequence of amino acids ISNGTSGGATNDNT (SEQ ID NO:46) at positions 261-274, the sequence of amino acids STGGTQGTQQLLFSQAGPANMSA (SEQ ID NO:36) at positions 453-475, and the sequence of amino acids KTGATNKTTLENVLMTNEEEIRP (SEQ ID NO:39) at positions 548-569, with numbering relative to SEQ ID NO:7; or sequences having 1, 2, 3, 4 or 5 conservative amino acid substitutions within the aforementioned sequences.

Capsid polypeptides of the disclosure also include those comprising all or a portion of the VP1 protein set forth in SEQ ID NO:8 (also referred to as AAV7-EE) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-737 of SEQ ID NO:8 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-737 of SEQ ID NO:8 or a functional fragment thereof; and capsid polypeptides comprising all or a portion of the VP3 protein set forth as amino acids 204-737 of SEQ ID NO:8 or comprising a sequence having at least or about 85%, 86%, 87%, 88%, 89%, 90%, 91%, 92%, 93%, 940%, 95%, 96%, 97%, 98%, or 99% sequence identity to the VP3 protein set forth as amino acids 204-737 of SEQ ID NO:8 or a functional fragment thereof. In particular embodiments, the capsid polypeptide comprises the VR-I from AAV8 and the VR-IV and VR-VII from AAVC11.12 (i.e. the VR-IV from AAV10 and the VR-VII from AAV7). In particular examples, the capsid polypeptide comprises amino acid residues N263, G264, T265, S266, G267, T270, T274, S453, Q458, G459, Q462, L464, A468, A471, N472, S474, A475, T548, G549, T551, N552, K553, T554, T555, L556, E557, N558, L560, M561, N563, R568 and P569, with numbering relative to SEQ ID NO:8. In one example, the capsid polypeptide comprises the sequence of amino acids NGTSGGATNDNT (SEQ ID NO:44) at positions 263-274, the sequence of amino acids STGGTQGTQQLLFSQAGPANMSA (SEQ ID NO: 36) at positions 453-475, and the sequence of amino acids TGATNKTTLENVLMTNEEEIRP (SEQ ID NO:33) at positions 548-569, with numbering relative to SEQ ID NO:8; or sequences having 1, 2, 3, 4 or 5 conservative amino acid substitutions within the aforementioned sequences. In a further example, the capsid polypeptide comprises the sequence of amino acids ISNGTSGGATNDNT (SEQ ID NO:46) at positions 261-274, the sequence of amino acids STGGTQGTQQLLFSQAGPANMSA (SEQ ID NO:36) at positions 453-475, and the sequence of amino acids KTGATNKTTLENVLMTNEEEIRP (SEQ ID NO:39) at positions 548-569, with numbering relative to SEQ ID NO:8; or sequences having 1, 2, 3, 4 or 5 conservative amino acid substitutions within the aforementioned sequences.

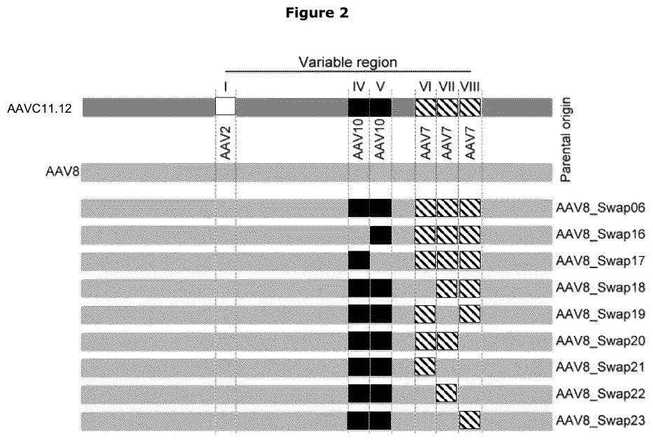

Provided are capsid polypeptides that comprise (i) the sequence of amino acids set forth in any one of SEQ ID NOs:9-16 (e.g. AAV-Swap16 to AAV-Swap23), or a sequence having at least or about 85%, 90% 95%, 96%, 97%, 98% or 99% sequence identity thereto; (ii) the sequence of amino acids at positions 138-737 of any one of SEQ ID NOs:9-11, 13 and 15, or positions 138-738 of any one of SEQ ID NOs:12, 14 and 16; or a sequence having at least or about 85%, 90%, 95%, 96%, 97%, 98% or 99% sequence identity thereto; and/or (iii) the sequence of amino acids at positions 204-737 of any one of SEQ ID NOs: 9-11, 13 and 15, or positions 204-738 of any one of SEQ ID NOs: 12, 14 and 16; or a sequence having at least or about 85%, 90%, 95%, 96%, 97%, 98% or 99% sequence identity thereto. In one example, the capsid polypeptides comprise amino acid residues S451, Q456, G457, Q460, L462, A466, A469, N470, S472 and A473 (i.e. including residues in and/or near the VR-IV of AAVC11.12); amino acid residues L493, S494, G505, A506, V518 and V522 (i.e. including residues in or near the VR-V of AAVC11.12); amino acid residues D532, S538 and V540 (i.e. including residues in or near the VR-VI of AAVC11.12); amino acid residues T546, G547, T549, N550, K551, T552, T553, L554, E555, N556, L558, M559, N561, R566 and P567 (i.e. including residues in or near the VR-VII of AAVC11.12); and/or amino acid residues S580, S581, A585, A586, A590, T592, Q593, V594, and N597 (i.e. including residues in or near the VR-VIII of AAVC11.12); with numbering relative to SEQ ID NO:4. In particular examples, polypeptide comprises the aforementioned residues in the combinations present in any one of AAV-Swap16, AAV-Swap17, AAV-Swap18, AAV-Swap19, AAV-Swap20, AAV-Swap21, AAV-Swap22 or AAV-Swap23, as shown in FIG. 2 and as depicted in the sequences set forth in SEQ ID NOs:9-16).

In further examples, these capsid polypeptides comprise the sequence of amino acids STGGTQGTQQLLFSQAGPANMSA (SEQ ID NO:36) at positions 451-473; the sequence of amino acids QSTGGTQGTQQLLFSQAGPANMSA (SEQ ID NO:41) at positions 450-473; the sequence of amino acids LSQNNNSNFAWTGATKYHLNGRNSLVNPGV (SEQ ID NO:37) at positions 493-522; the sequence of amino acids RVSTTLSQNNNSNFAWTGATKYHLNGRNSLVNPGV (SEQ ID NO:42) at positions 488-522; the sequence of amino acids DRFFPSSGV (SEQ ID NO:35) at positions 532-540; the sequence of amino acids AMATHKDDEDRFFPSSGV (SEQ ID NO:40) at positions 523-540; the sequence of amino acids TGATNKTTLENVLMTNEEEIRP (SEQ ID NO:33) at positions 546-567; the sequence of amino acids KTGATNKTTLENVLMTNEEEIRP (SEQ ID NO:39) at positions 545-567; and/or the sequence of amino acids SSNLQAANTAAQTQVVNN (SEQ ID NO:34) at positions 580-597; with numbering relative to SEQ ID NO:4 (e.g. in the combinations present in AAV-Swap16 to AAV-Swap23, as shown in FIG. 2 and as present in SEQ ID Nos:9-16). In some embodiments, the capsid polypeptides comprise a sequence of amino acids having at least about 50%, 60%, 70%, 80%, or 90% sequence identity to SEQ ID Nos:32-42 and have 1, 2, 3, 4, 5, 6, 6, 8, or 9 conservative amino acid substitutions of any residue SEQ ID Nos:32-42.

Also provided are capsid polypeptides that are variants of the AAVC11.12 capsid (SEQ ID NO:4) and which comprise at least 6-17 amino acid modifications (e.g. substitutions and/or insertions) in the VR-IV, VR-VII and/or VR-III relative to the AAVC11.12 capsid sequence. These include capsids C11.12.253R4.1, C11.12.253R4.3, C11.12.253R4.6, C11.12.253R4.9, C11.12.269R4.1, C11.12.269R4.3, C11.12.269R4.5, C11.12.269R4.6, and C11.12.269R4.20, and variants thereof, as described below. In some examples, these capsid polypeptides retain all or a portion of the VR-I, VR-V and VR-VI of C11.12, e.g. comprise (i) residues in or near the VR-I of AAVC11.12, such as amino acid residues S263, Q264, S265, S268 and H272, the sequence of amino acids SQSGASNDNH (SEQ ID NO:32) at positions 263-272, and/or the sequence of amino acids ISSQSGASNDNH (SEQ ID NO:38) at positions 261-272, with numbering relative to SEQ ID NO:4; (ii) amino acid residues in or near the VR-V of AAVC11.12, such as amino acid residues L493, S494, G505, A506, V518 and V522, the sequence of amino acids LSQNNNSNFAWTGATKYHLNGRNSLVNPGV (SEQ ID NO:37) at positions 493-522, and/or the sequence of amino acids RVSTTLSQNNNSNFAWTGATKYHLNGRNSLVNPGV (SEQ ID NO:42) at positions 488-522, with numbering relative to SEQ ID NO:4; and/or (iii) comprises residues in or near the VR-VI of AAVC11.12, such as amino acid residues D532, S538 and V540, the sequence of amino acids DRFFPSSGV (SEQ ID NO:35) at positions 532-540; and/or the sequence of amino acids AMATHKDDEDRFFPSSGV (SEQ ID NO:40) at positions 523-540, with numbering relative to SEQ ID NO:4.

In one example, the capsid polypeptide comprises all or a portion of the VP1 protein set forth in SEQ ID NO:47 (also referred to as C11.12.253R4.1) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO: 47 or comprising a sequence having at least or about 85%, 86%, 87%, 88%, 89%, 90%, 91%, 92%, 93%, 940%, 95%, 96%, 97%, 98%, or 99% sequence identity to the VP2 protein set forth as amino acids 138-735 of SEQ ID NO: 47 or a functional fragment thereof; and capsid polypeptides comprising all or a portion of the VP3 protein set forth as amino acids 204-735 of SEQ ID NO: 47 or comprising a sequence having at least or about 85%, 86%, 87%, 88%, 89%, 90%, 91%, 92%, 93%, 940%, 95%, 96%, 97%, 98%, or 99% sequence identity to the VP3 protein set forth as amino acids 204-735 of SEQ ID NO:47 or a functional fragment thereof. In particular embodiments, the capsid polypeptide comprises the following amino acid substitutions in the VR-VII or adjacent region, relative to C11.12: T546Q, A548S, T549A, K551D, T552A, L554H, N556D, L558M, M559L, and N561D, i.e. the capsid polypeptide comprises amino acid residues Q546, S548, A549, D551, A552, H554, D556, M558, L559, and D561, with numbering relative to SEQ ID NO:47. In one example, the capsid polypeptide comprises the sequence of amino acids QGSANDATHEDVMLTD (SEQ ID NO:65) at positions 546-561, and/or KQGSANDATHEDVMLTDEEEIRP (SEQ ID NO:66) at positions 545-567 with numbering relative to SEQ ID NO:47, or sequences having 1, 2, 3, 4 or 5 conservative amino acid substitutions of amino acid residues within the aforementioned sequences. In particular embodiments, the capsid polypeptides comprise all or a portion of the VR-IV and VR-VIII of C11.12, e.g. amino acid residues S451, Q456, G457, Q460, L462, A466, A469, N470, S472 and A473 (i.e. in and/or near the VR-IV of AAVC11.12) and/or amino acid residues S580, S581, A585, A586, A590, T592, Q593, V594, and N597 (i.e. in or near the VR-VIII of AAVC11.12); with numbering relative to SEQ ID NO:4. In some embodiments, the capsid polypeptides comprise the sequence of amino acids STGGTQGTQQLLFSQAGPANMSA (SEQ ID NO:36) at positions 451-473; the sequence of amino acids QSTGGTQGTQQLLFSQAGPANMSA (SEQ ID NO:41) at positions 450-473; and/or the sequence of amino acids SSNLQAANTAAQTQVVNN (SEQ ID NO:34) at positions 580-597, with numbering relative to SEQ ID NO:4, or sequences having 1, 2, 3, 4 or 5 conservative amino acid substitutions of amino acid residues within the aforementioned sequences.

In another example, provided is a capsid polypeptide comprising all or a portion of the VP1 protein set forth in SEQ ID NO:48 (also referred to as C11.12.253R4.3) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO: 48 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO: 48 or a functional fragment thereof; and capsid polypeptides comprising all or a portion of the VP3 protein set forth as amino acids 204-735 of SEQ ID NO: 48 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 204-735 of SEQ ID NO:48 or a functional fragment thereof. In particular embodiments, the capsid polypeptide comprises the following amino acid substitutions in the VR-VIII or adjacent region, relative to C11.12: A585N, A586S, A590P, Q591E, T592I, Q593A, V594N, i.e. the capsid polypeptide comprises amino acid residues N585, S586, P590, E591, 1592, A593 and N594, with numbering relative to SEQ ID NO:48. In one example, the capsid polypeptide comprises the sequence of amino acids NSNTAPEIAN (SEQ ID NO:67) at positions 585-594, and/or SSNLQNSNTAPEIANVNN (SEQ ID NO:68) at positions 580-597 with numbering relative to SEQ ID NO:48, or a sequence having 1, 2, 3, 4 or 5 conservative amino acid substitutions of amino acid residues within the aforementioned sequences. In particular embodiments, the capsid polypeptides comprise all or a portion of the VR-IV and VR-VII of C11.12, e.g. amino acid residues S451, Q456, G457, Q460, L462, A466, A469, N470, S472 and A473 (i.e. in and/or near the VR-IV of AAVC11.12) and/or amino acid residues T546, G547, T549, N550, K551, T552, T553, L554, E555, N556, L558, M559, N561, R566 and P567 (i.e. in or near the VR-VII of AAVC11.12); with numbering relative to SEQ ID NO:4. In some embodiments, the capsid polypeptides comprise the sequence of amino acids STGGTQGTQQLLFSQAGPANMSA (SEQ ID NO:36) at positions 451-473; the sequence of amino acids QSTGGTQGTQQLLFSQAGPANMSA (SEQ ID NO:41) at positions 450-473; the sequence of amino acids TGATNKTTLENVLMTNEEEIRP (SEQ ID NO:33) at positions 546-567; and/or the sequence of amino acids KTGATNKTTLENVLMTNEEEIRP (SEQ ID NO:39) at positions 545-567; with numbering relative to SEQ ID NO:4, or sequences having 1, 2, 3, 4 or 5 conservative amino acid substitutions of amino acid residues within the aforementioned sequences.

Also provided is a capsid polypeptide comprising all or a portion of the VP1 protein set forth in SEQ ID NO:49 (also referred to as C11.12.253R4.6) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO: 49 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO: 49 or a functional fragment thereof; and capsid polypeptides comprising all or a portion of the VP3 protein set forth as amino acids 204-735 of SEQ ID NO: 49 or comprising a sequence having at least or about 85%, 86%, 87%, 88%, 89%, 90%, 91%, 92%, 93%, 940%, 95%, 96%, 97%, 98%, or 99% sequence identity to the VP3 protein set forth as amino acids 204-735 of SEQ ID NO:49 or a functional fragment thereof. In particular embodiments, the capsid polypeptide comprises the following amino acid substitutions in the VR-VR-VII and VR-VIII or adjacent regions, relative to C11.12: T546Q, A548S, T549A, K551D, T552A, L554H, N556D, L558M, M559L, N561D, A585N, A586S, A590P, Q591E, T592I, Q593A and V594N, i.e. the capsid polypeptide comprises amino acid residues Q546, S548, A549, D551, A552, H554, D556, M558, L559, D561, N585, S586, P590, E591, 1592, A593 and N594, with numbering relative to SEQ ID NO:49. In one example, the capsid polypeptide comprises the sequence of amino acids QGSANDATHEDVMLTD (SEQ ID NO:65) at positions 546-561, the sequence of amino acids KQGSANDATHEDVMLTDEEEIRP (SEQ ID NO:66) at positions 545-567, the sequence of amino acids NSNTAPEIAN (SEQ ID NO:67) at positions 585-594, and/or the sequence of amino acids SSNLQNSNTAPEIANVNN (SEQ ID NO:68) at positions 580-597 with numbering relative to SEQ ID NO:49, or sequences having 1, 2, 3, 4 or 5 conservative amino acid substitutions of amino acid residues within the aforementioned sequences. In particular embodiments, the capsid polypeptides comprise all or a portion of the VR-IV of C11.12, e.g. amino acid residues S451, Q456, G457, Q460, L462, A466, A469, N470, S472 and A473 (i.e. in and/or near the VR-IV of AAVC11.12) with numbering relative to SEQ ID NO:4. In some embodiments, the capsid polypeptides comprise the sequence of amino acids STGGTQGTQQLLFSQAGPANMSA (SEQ ID NO:36) at positions 451-473 and/or the sequence of amino acids QSTGGTQGTQQLLFSQAGPANMSA (SEQ ID NO:41) at positions 450-473, or sequences having 1, 2, 3, 4 or 5 conservative amino acid substitutions of amino acid residues within the aforementioned sequences.

Also provided is a capsid polypeptide comprising all or a portion of the VP1 protein set forth in SEQ ID NO:50 (also referred to as C11.12.253R4.9) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-736 of SEQ ID NO: 50 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-736 of SEQ ID NO: 50 or a functional fragment thereof; and capsid polypeptides comprising all or a portion of the VP3 protein set forth as amino acids 204-736 of SEQ ID NO: 50 or comprising a sequence having at least or about 85%, 86%, 87%, 88%, 89%, 90%, 91%, 92%, 93%, 940%, 95%, 96%, 97%, 98%, or 99% sequence identity to the VP3 protein set forth as amino acids 204-736 of SEQ ID NO:50 or a functional fragment thereof. In particular embodiments, the capsid polypeptide comprises the following amino acid modifications in the VR-VII or adjacent region, relative to C11.12: T546K, G547N, A548T, T549N, T549_N550insT, K551N, T552V, T553N, L554Y and L558M, i.e. the capsid polypeptide comprises amino acid residues K546, N547, T548, N549, T550, N552, V553, N554, Y555 and M559, with numbering relative to SEQ ID NO:47. In one example, the capsid polypeptide comprises the sequence of amino acids KNTNTNNVNYENVMM (SEQ ID NO:69) at positions 546-559, and/or KKNTNTNNVNYENVMMTNEEEIRP (SEQ ID NO:70) at positions 545-568 with numbering relative to SEQ ID NO:50, or sequences having 1, 2, 3, 4 or 5 conservative amino acid substitutions of amino acid residues within the aforementioned sequences. In particular embodiments, the capsid polypeptides comprise all or a portion of the VR-IV and VR-VIII of C11.12, e.g. amino acid residues S451, Q456, G457, Q460, L462, A466, A469, N470, S472 and A473 (i.e. in and/or near the VR-IV of AAVC11.12) and/or amino acid residues S580, S581, A585, A586, A590, T592, Q593, V594, and N597 (i.e. in or near the VR-VIII of AAVC11.12); with numbering relative to SEQ ID NO:4. In some embodiments, the capsid polypeptides comprise the sequence of amino acids STGGTQGTQQLLFSQAGPANMSA (SEQ ID NO:36) at positions 451-473; the sequence of amino acids QSTGGTQGTQQLLFSQAGPANMSA (SEQ ID NO:41) at positions 450-473; and/or the sequence of amino acids SSNLQAANTAAQTQVVNN (SEQ ID NO:34) at positions 580-597, with numbering relative to SEQ ID NO:4, or sequences having 1, 2, 3, 4 or 5 conservative amino acid substitutions of amino acid residues within the aforementioned sequences.

Also provided is a capsid polypeptide comprising all or a portion of the VP1 protein set forth in SEQ ID NO:51 (also referred to as C11.12.269R4.1) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-736 of SEQ ID NO: 51 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-736 of SEQ ID NO: 51 or a functional fragment thereof; and capsid polypeptides comprising all or a portion of the VP3 protein set forth as amino acids 204-736 of SEQ ID NO: 51 or comprising a sequence having at least or about 85%, 86%, 87%, 88%, 89%, 90%, 91%, 92%, 93%, 940%, 95%, 96%, 97%, 98%, or 99% sequence identity to the VP3 protein set forth as amino acids 204-736 of SEQ ID NO:51 or a functional fragment thereof. In particular embodiments, the capsid polypeptide comprises the following amino acid modifications in the VR-IV or adjacent region, relative to C11.12: S447G, S451A, T452_G453insP, Q456T, T458Q, Q459N and Q460T, i.e. the polypeptide comprises amino acid residues G447, A451, P453, T457, Q459, N460 and T461, with numbering relative to SEQ ID NO:51. In one example, the capsid polypeptide comprises the sequence of amino acids GRTQATPGGTTGQNT (SEQ ID NO:71) at positions 447-461, and/or GRTQATPGGTTGQNTLLFSQAGPANMSA (SEQ ID NO:72) at positions 447-474 with numbering relative to SEQ ID NO:51, or sequences having 1, 2, 3, 4 or 5 conservative amino acid substitutions of amino acid residues within the aforementioned sequences. In particular embodiments, the capsid polypeptides comprise all or a portion of the VR-VII and VR-VIII of C11.12, e.g. amino acid residues T546, G547, T549, N550, K551, T552, T553, L554, E555, N556, L558, M559, N561, R566 and P567 (i.e. in or near the VR-VII of AAVC11.12); and/or amino acid residues S580, S581, A585, A586, A590, T592, Q593, V594, and N597 (i.e. in or near the VR-VIII of AAVC11.12) with numbering relative to SEQ ID NO:4. In some embodiments, the capsid polypeptides comprise the sequence of amino acids TGATNKTTLENVLMTNEEEIRP (SEQ ID NO:33) at positions 546-567; the sequence of amino acids KTGATNKTTLENVLMTNEEEIRP (SEQ ID NO:39) at positions 545-567; and/or the sequence of amino acids SSNLQAANTAAQTQVVNN (SEQ ID NO:34) at positions 580-597, with numbering relative to SEQ ID NO:4, or sequences having 1, 2, 3, 4 or 5 conservative amino acid substitutions of amino acid residues within the aforementioned sequences.

In another example, provided is a capsid polypeptide comprising all or a portion of the VP1 protein set forth in SEQ ID NO:52 (also referred to as C11.12.269R4.3) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO: 52 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO: 52 or a functional fragment thereof; and capsid polypeptides comprising all or a portion of the VP3 protein set forth as amino acids 204-735 of SEQ ID NO: 52 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 204-735 of SEQ ID NO:52 or a functional fragment thereof. In particular embodiments, the capsid polypeptide comprises the following amino acid substitutions in the VR-VIII or adjacent region, relative to C11.12: A585K, A586E, A589P, T592I, Q593E and V594A, i.e. the capsid polypeptide comprises amino acid residues K585, E586, P589, 1592, E593 and A594, with numbering relative to SEQ ID NO:52. In one example, the capsid polypeptide comprises the sequence of amino acids KENTPAQIEA (SEQ ID NO:73) at positions 585-594, and/or SSNLQKENTPAQIEAVNN (SEQ ID NO:74) at positions 580-597 with numbering relative to SEQ ID NO:52, or sequences having 1, 2, 3, 4 or 5 conservative amino acid substitutions of amino acid residues within the aforementioned sequences. In particular embodiments, the capsid polypeptides comprise all or a portion of the VR-IV and VR-VII of C11.12, e.g. amino acid residues S451, Q456, G457, Q460, L462, A466, A469, N470, S472 and A473 (i.e. in and/or near the VR-IV of AAVC11.12) and/or amino acid residues T546, G547, T549, N550, K551, T552, T553, L554, E555, N556, L558, M559, N561, R566 and P567 (i.e. in or near the VR-VII of AAVC11.12); with numbering relative to SEQ ID NO:4. In some embodiments, the capsid polypeptides comprise the sequence of amino acids STGGTQGTQQLLFSQAGPANMSA (SEQ ID NO:36) at positions 451-473; the sequence of amino acids QSTGGTQGTQQLLFSQAGPANMSA (SEQ ID NO:41) at positions 450-473; the sequence of amino acids TGATNKTTLENVLMTNEEEIRP (SEQ ID NO:33) at positions 546-567; and/or the sequence of amino acids KTGATNKTTLENVLMTNEEEIRP (SEQ ID NO:39) at positions 545-567; with numbering relative to SEQ ID NO:4, or sequences having 1, 2, 3, 4 or 5 conservative amino acid substitutions of amino acid residues within the aforementioned sequences.

In another example, provided is a capsid polypeptide comprising all or a portion of the VP1 protein set forth in SEQ ID NO:53 (also referred to as C11.12.269R4.5) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-736 of SEQ ID NO: 53 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-736 of SEQ ID NO: 53 or a functional fragment thereof; and capsid polypeptides comprising all or a portion of the VP3 protein set forth as amino acids 204-736 of SEQ ID NO: 53 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 204-736 of SEQ ID NO:53 or a functional fragment thereof. In particular embodiments, the capsid polypeptide comprises the following amino acid modifications in the VR-VII or adjacent region, relative to C11.12: T546Q, G547S, A548S, T549S, T549_N550insG, N550A, T552V, T553N, N556G, M559L and N561G, i.e. the capsid polypeptide comprises amino acid residues Q546, S547, S548, S549, G550, A551, V553, N554, G557, L560 and G562 with numbering relative to SEQ ID NO:53. In one example, the capsid polypeptide comprises the sequence of amino acids QSSSGAKVNLEGVLLTG (SEQ ID NO:75) at positions 546-562, and/or KQSSSGAKVNLEGVLLTGEEEIRP (SEQ ID NO:76) at positions 545-568 with numbering relative to SEQ ID NO:53, or sequences having 1, 2, 3, 4 or 5 conservative amino acid substitutions of amino acid residues within the aforementioned sequences. In particular embodiments, the capsid polypeptides comprise all or a portion of the VR-IV and VR-VIII of C11.12, e.g. amino acid residues S451, Q456, G457, Q460, L462, A466, A469, N470, S472 and A473 (i.e. in and/or near the VR-IV of AAVC11.12) and/or amino acid residues S580, S581, A585, A586, A590, T592, Q593, V594, and N597 (i.e. in or near the VR-VIII of AAVC11.12); with numbering relative to SEQ ID NO:4. In some embodiments, the capsid polypeptides comprise the sequence of amino acids STGGTQGTQQLLFSQAGPANMSA (SEQ ID NO:36) at positions 451-473; the sequence of amino acids QSTGGTQGTQQLLFSQAGPANMSA (SEQ ID NO:41) at positions 450-473; and/or the sequence of amino acids SSNLQAANTAAQTQVVNN (SEQ ID NO:34) at positions 580-597, with numbering relative to SEQ ID NO:4, or sequences having 1, 2, 3, 4 or 5 conservative amino acid substitutions of amino acid residues within the aforementioned sequences.

Also provided is a capsid polypeptide comprising all or a portion of the VP1 protein set forth in SEQ ID NO:54 (also referred to as C11.12.269R4.6) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO: 54 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO: 54 or a functional fragment thereof; and capsid polypeptides comprising all or a portion of the VP3 protein set forth as amino acids 204-735 of SEQ ID NO: 54 or comprising a sequence having at least or about 85%, 86%, 87%, 88%, 89%, 90%, 91%, 92%, 93%, 940%, 95%, 96%, 97%, 98%, or 99% sequence identity to the VP3 protein set forth as amino acids 204-735 of SEQ ID NO:54 or a functional fragment thereof. In particular embodiments, the capsid polypeptide comprises the following amino acid substitutions in the VR-IV, relative to C11.12: R448K, S451A, G453S, Q456K, G457D and Q459N, i.e. the polypeptide comprises amino acid residues K448, A451, S453, K456, D457 and N459, with numbering relative to SEQ ID NO:54. In one example, the capsid polypeptide comprises the sequence of amino acids KTQATSGTKDTN (SEQ ID NO:77) at positions 448-459, and/or KTQATSGTKDTNQLLFSQAGPANMSA (SEQ ID NO:78) at positions 448-473 with numbering relative to SEQ ID NO:54, or sequences having 1, 2, 3, 4 or 5 conservative amino acid substitutions of amino acid residues within the aforementioned sequences. In particular embodiments, the capsid polypeptides comprise all or a portion of the VR-VII and VR-VIII of C11.12, e.g. amino acid residues T546, G547, T549, N550, K551, T552, T553, L554, E555, N556, L558, M559, N561, R566 and P567 (i.e. in or near the VR-VII of AAVC11.12); and/or amino acid residues S580, S581, A585, A586, A590, T592, Q593, V594, and N597 (i.e. in or near the VR-VIII of AAVC11.12) with numbering relative to SEQ ID NO:4. In some embodiments, the capsid polypeptides comprise the sequence of amino acids TGATNKTTLENVLMTNEEEIRP (SEQ ID NO:33) at positions 546-567; the sequence of amino acids KTGATNKTTLENVLMTNEEEIRP (SEQ ID NO:39) at positions 545-567; and/or the sequence of amino acids SSNLQAANTAAQTQVVNN (SEQ ID NO:34) at positions 580-597, with numbering relative to SEQ ID NO:4, or sequences having 1, 2, 3, 4 or 5 conservative amino acid substitutions of amino acid residues within the aforementioned sequences.

In another example, provided is a capsid polypeptide comprising all or a portion of the VP1 protein set forth in SEQ ID NO:55 (also referred to as C11.12.269R4.20) or a polypeptide having at least or about 85%, 86%, 87%, 88%, 89%, 90%, 91%, 92%, 93%, 94%, 95%, 96%, 97%, 98%, or 99% sequence identity thereto. Thus, also included in the present disclosure are capsid polypeptides comprising all or a portion of the VP2 protein set forth as amino acids 138-735 of SEQ ID NO: 55 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO: 55 or a functional fragment thereof; and capsid polypeptides comprising all or a portion of the VP3 protein set forth as amino acids 204-735 of SEQ ID NO: 55 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 204-735 of SEQ ID NO:55 or a functional fragment thereof. In particular embodiments, the capsid polypeptide comprises the following amino acid substitutions in the VR-VIII, relative to C11.12: A585T, A586G, A590P, Q591E, Q593L, V594I and N597H, i.e. the capsid polypeptide comprises amino acid residues T585, G586, P590, E591, L593, 1594 and H597 with numbering relative to SEQ ID NO:55. In one example, the capsid polypeptide comprises the sequence of amino acids TGNTAPETLIVNH (SEQ ID NO:79) at positions 585-597, and/or SSNLQTGNTAPETLIVNH (SEQ ID NO:80) at positions 580-597, with numbering relative to SEQ ID NO:55, or sequences having 1, 2, 3, 4 or 5 conservative amino acid substitutions of amino acid residues within the aforementioned sequences. In particular embodiments, the capsid polypeptides comprise all or a portion of the VR-IV and VR-VII of C11.12, e.g. amino acid residues S451, Q456, G457, Q460, L462, A466, A469, N470, S472 and A473 (i.e. in and/or near the VR-IV of AAVC11.12) and/or amino acid residues T546, G547, T549, N550, K551, T552, T553, L554, E555, N556, L558, M559, N561, R566 and P567 (i.e. in or near the VR-VII of AAVC11.12); with numbering relative to SEQ ID NO:4. In some embodiments, the capsid polypeptides comprise the sequence of amino acids STGGTQGTQQLLFSQAGPANMSA (SEQ ID NO:36) at positions 451-473; the sequence of amino acids QSTGGTQGTQQLLFSQAGPANMSA (SEQ ID NO:41) at positions 450-473; the sequence of amino acids TGATNKTTLENVLMTNEEEIRP (SEQ ID NO:33) at positions 546-567; and/or the sequence of amino acids KTGATNKTTLENVLMTNEEEIRP (SEQ ID NO:39) at positions 545-567; with numbering relative to SEQ ID NO:4, or sequences having 1, 2, 3, 4 or 5 conservative amino acid substitutions of amino acid residues within the aforementioned sequences.

Also provided are nucleic acid molecules, including isolated nucleic acid molecules, encoding a capsid polypeptide described herein. Thus, for example, amongst the nucleic acid molecules provided herein are those encoding the VP1, VP2 and/or VP3 of any one of the capsid polypeptides described herein. Non-limiting examples of nucleic acid molecules therefore include those set forth in SEQ ID NOs:20-31 and 56-64, those having at least or about 85%, 86%, 87%, 88%, 89%, 90%, 91%, 92%, 93%, 94%, 95%, 96%, 97%, 98%, or 99% sequence identity thereto, and those that hybridize with medium or high stringency to nucleic acid molecules comprising a sequence set forth in any one of SEQ ID NOs:20-31 and 56-64.

### Vectors

The present disclosure also provides vectors comprising a nucleic acid molecule that encodes a capsid polypeptide described herein, and vectors comprising a capsid polypeptide described herein. The vectors include nucleic acid vectors that comprise a nucleic acid molecule that encodes a capsid polypeptide described herein, and AAV vectors that have a capsid comprising a capsid polypeptide described herein.

### Nucleic Acid Vectors

Vectors of the present disclosure include nucleic acid vectors that comprise a polynucleotide that encodes all or a portion of a capsid polypeptide described herein, e.g. that encodes a polypeptide comprising an amino acid sequence set forth in any one of SEQ ID NOs:5-16 or an amino acid sequence having at least or about 85%, 86%, 87%, 88%, 89%, 90%, 91%, 92%, 93%, 94%, 95%, 96%, 97%, 98% or 99% sequence identity to a sequence set forth in any one of SEQ ID NOs:5-16 and 47-55, or a fragment thereof (e.g. all or a portion of the VP2 or VP3 protein), as described above. The vectors can be episomal vectors (i.e., that do not integrate into the genome of a host cell) or can be vectors that integrate into the host cell genome. Exemplary vectors that comprise a nucleic acid molecule encoding a capsid polypeptide include, but are not limited to, plasmids, cosmids, transposons and artificial chromosomes. In particular examples, the vectors are plasmids.

Vectors, such as plasmids, suitable for use in bacterial, insect and mammalian cells are widely described and well-known in the art. Those skilled in the art would appreciate that vectors of the present disclosure may also contain additional sequences and elements useful for the replication of the vector in prokaryotic and/or eukaryotic cells, selection of the vector and the expression of a heterologous sequence in a variety of host cells. For example, the vectors of the present disclosure can include a prokaryotic replicon (that is, a sequence having the ability to direct autonomous replication and maintenance of the vector extra-chromosomally in a prokaryotic host cell, such as a bacterial host cell. Such replicons are well known in the art. In some embodiments, the vectors can include a shuttle element that makes the vectors suitable for replication and integration in both prokaryotes and eukaryotes. In addition, vectors may also include a gene whose expression confers a detectable marker such as a drug resistance gene, which allows for selection and maintenance of the host cells. Vectors may also have a reportable marker, such as gene encoding a fluorescent or other detectable protein. The nucleic acid vectors will likely also comprise other elements, including any one or more of those described below. Most typically, the vectors will comprise a promoter operably linked to the nucleic acid encoding the capsid protein.

The nucleic acid vectors of the present disclosure can be constructed using known techniques, including, without limitation, the standard techniques of restriction endonuclease digestion, ligation, transformation, plasmid purification, in vitro or chemical synthesis of DNA, and DNA sequencing. The vectors of the present disclosure may be introduced into a host cell using any method known in the art. Accordingly, the present disclosure is also directed to host cells comprising a vector or nucleic acid described herein.

### AAV Vectors

Provided herein are AAV vectors comprising a capsid polypeptide described herein, such as a polypeptide comprising all or a portion of an AAV capsid protein (e.g. a polypeptide comprising the amino acid sequence set forth in any one of SEQ ID NOs:5-16 and 47-55 or an amino acid sequence having at least or about 85%, 86%, 87%, 88%, 89%, 90%, 91%, 92%, 93%, 94%, 95%, 96%, 97%, 98% or 99% sequence identity to a sequence set forth in any one of SEQ ID NOs:5-16, or a fragment thereof (e.g. all or a portion of the VP2 or VP3 protein).

Methods for vectorizing a capsid protein are well known in the art and any suitable method can be employed for the purposes of the present disclosure. For example, the cap gene can be recovered (e.g. by PCR or digest with enzymes that cut upstream and downstream of cap) and cloned into a packaging construct containing rep. Any AAV rep gene may be used, including, for example, a rep gene is from AAV1, AAV2, AAV3, AAV4, AAV5, AAV6, AAV7, AAV8, AAV9, AAV10, AAV11, AAV12 or AAV13 and any variants thereof. Typically, the cap gene is cloned downstream of rep so the rep p40 promoter can drive cap expression. This construct does not contain ITRs. This construct is then introduced into a packaging cell line with a second construct containing ITRs, typically flanking a heterologous coding sequence. Helper function or a helper virus are also introduced, and recombinant AAV comprising a capsid generated from capsid proteins expressed from the cap gene, and encapsidating a genome comprising the transgene flanked by the ITRs, is recovered from the supernatant of the packaging cell line. Various types of cells can be used as the packaging cell line. For example, packaging cell lines that can be used include, but are not limited to, HEK293 cells, HeLa cells, and Vero cells, for example as disclosed in US20110201088. The helper functions may be provided by one or more helper plasmids or helper viruses comprising adenoviral helper genes. Non-limiting examples of the adenoviral helper genes include E1A, E1B, E2A, E4 and VA, which can provide helper functions to AAV packaging. Helper viruses of AAV are known in the art and include, for example, viruses from the family Adenoviridae and the family Herpesviridae. Examples of helper viruses of AAV include, but are not limited to, SAdV-13 helper virus and SAdV-13-like helper virus described in US20110201088, helper vectors pHELP (Applied Viromics). A skilled artisan will appreciate that any helper virus or helper plasmid of AAV that can provide adequate helper function to AAV can be used herein.

In some instances, rAAV virions are produced using a cell line that stably expresses some of the necessary components for AAV virion production. For example, a plasmid (or multiple plasmids) comprising the nucleic acid containing a cap gene identified as described herein and a rep gene, and a selectable marker, such as a neomycin resistance gene, can be integrated into the genome of a cell (the packaging cells). The packaging cell line can then be transfected with an AAV vector and a helper plasmid or transfected with an AAV vector and co-infected with a helper virus (e.g., adenovirus providing the helper functions). The advantages of this method are that the cells are selectable and are suitable for large-scale production of the recombinant AAV. As another non-limiting example, adenovirus or baculovirus rather than plasmids can be used to introduce the nucleic acid encoding the capsid polypeptide, and optionally the rep gene, into packaging cells. As yet another non-limiting example, the AAV vector is also stably integrated into the DNA of producer cells, and the helper functions can be provided by a wild-type adenovirus to produce the recombinant AAV.

In still further instances, the AAV vectors are produced synthetically, by synthesising AAV capsid proteins and assembling and packaging the capsids in vitro.

Typically, the AAV vectors of the present disclosure also comprise a heterologous coding sequence. The heterologous coding sequence may be operably linked to a promoter to facilitate expression of the sequence. The heterologous coding sequence can encode a peptide or polypeptide, such as a therapeutic peptide or polypeptide, or can encode a polynucleotide or transcript that itself has a function or activity, such as an antisense or inhibitory oligonucleotide, including antisense DNA and RNA (e.g. miRNA, siRNA, and shRNA). In some examples, the heterologous coding sequence is a stretch of nucleic acids that is essentially homologous to a stretch of nucleic acids in the genomic DNA of an animal, such that when the heterologous coding sequence is introduced into a cell of the animal, homologous recombination between the heterologous coding sequence and the genomic DNA can occur. As would be appreciated, the nature of the heterologous coding sequence is not essential to the present disclosure. In particular embodiments, the vectors comprising the heterologous coding sequence(s) will be used in gene therapy.

In particular examples, the heterologous coding sequence encodes a peptide or polypeptide, or polynucleotide, whose expression is of therapeutic use, such as, for example, for the treatment of a disease or disorder. For example, expression of a therapeutic peptide or polypeptide may serve to restore or replace the function of the endogenous form of the peptide or polypeptide that is defective (i.e. gene replacement therapy). In other examples, expression of a therapeutic peptide or polypeptide, or polynucleotide, from the heterologous sequence serves to alter the levels and/or activity of one or more other peptides, polypeptides or polynucleotides in the host cell. Thus, according to particular embodiments, the expression of a heterologous coding sequence introduced by a vector described herein into a host cell can be used to provide a therapeutic amount of a peptide, polypeptide or polynucleotide to ameliorate the symptoms of a disease or disorder. In other instance, the heterologous coding sequence is a stretch of nucleic acids that is essentially homologous to a stretch of nucleic acids in the genomic DNA of an animal, such that when the heterologous sequence is introduced into a cell of the animal, homologous recombination between the heterologous coding sequence and the genomic DNA can occur. Accordingly, the introduction of a heterologous sequence by an AAV vector described herein into a host cell can be used to correct mutations in genomic DNA, which in turn can ameliorate the symptoms of a disease or disorder.

In non-limiting examples, the heterologous coding sequence encodes an expression product that, when delivered to a subject, and in particular the liver of a subject, treats a liver-associated disease or condition. In illustrative embodiments, the liver-associated disease or condition is selected from among a urea cycle disorder (UCD; including N-acetylglutamate synthase deficiency (NAGSD), carbamylphosphate synthetase 1 deficiency (CPS1D), ornithine transcarbamylase deficiency (OTCD), argininosuccinate synthetase deficiency (ASSD), argininosuccinate lyase (ASLD), arginase 1 deficiency (ARG1D), citrin or aspartate/glutamate carrier deficiency and the mitochondrial ornithine transporter 1 deficiency causing hyperornithinemia-hyperammonemia-homocitrullinuria syndrome (HHH syndrome)), organic acidopathy (or organic academia, including methylmalonic acidemia, propionic acidemia, isovaleric acidemia, and maple syrup urine disease), aminoacidopathy, glycogenoses (Types I, III and IV), Wilson's disease, Progressive Familial Intrahepatic Cholestasis, primary hyperoxaluria, complementopathy, coagulopathy (e.g. hemophilia A, hemophilia B, von Willebrand disease (VWD)), Crigler Najjar syndrome, familial hypercholesterolaemia, α-1-antitrypsin deficiency, mitochondria respiratory chain hepatopathy, and citrin deficiency. Those skilled in the art would readily be able to select an appropriate heterologous coding sequence useful for treating such diseases. In some examples, the heterologous coding sequence comprises all or a part of a gene that is associated with the disease, such as all or a part of a gene set forth in Table 2. Introduction of such a sequence to the liver can be used for gene replacement or gene editing/correction, e.g. using CRISPR-Cas9. In particular examples, the heterologous coding sequence encodes a protein encoded by a gene that is associated with the disease, such as a gene set forth in Table 2.

TABLE 2

| Exemplary liver-associated diseases | Exemplary associated genes |
|---|---|
| Urea cycle disorders (UCDs) | OTC, ASS, CPS1, ASL, ARG1 |
| Organic acidopathies | PCCA, PCCB, MMUT |
| Aminoacidopathies | PAH, FAH |
| Glycogenoses (Types I, III and IV) | SLC37A4 |
| Wilson's Disease | ATP7B |
| Progressive Familial Intrahepatic | ABCB4, ABCB11, ATP8B1 |
| Cholestasis | |
| Primary Hyperoxaluria | AGXT |
| Complementopathies | CFH, CFI |
| Coagulopathies | F8, F9, VWF |
| Crigler Najjar syndrome | UGT1A1 |
| Familial Hypercholesterolaemia | LDLR |
| α-1-antitrypsin Deficiency | SERPINA1 |
| Mitochondria Respiratory Chain | POLG |
| Hepatopathies | |
| Citrin Deficiency | SLC25A13 |

The heterologous coding sequence in the AAV vector is flanked by 3′ and 5′ AAV ITRs. AAV ITRs used in the vectors of the disclosure need not have a wild-type nucleotide sequence, and may be altered, e.g., by the insertion, deletion or substitution of nucleotides. Additionally, AAV ITRs may be derived from any of several AAV serotypes, including without limitation, AAV1, AAV2, AAV3, AAV4, AAV5, AAV6, AAV7, AAV8, AAV9, AAV10, AAV11, AAV12 or AAV13. Such ITRs are well known in the art.

As will be appreciated by a skilled artisan, any method suitable for purifying AAV can be used in the embodiments described herein to purify the AAV vectors, and such methods are well known in the art. For example, the AAV vectors can be isolated and purified from packaging cells and/or the supernatant of the packaging cells. In some embodiments, the AAV is purified by separation method using a CsCl or iodixanol gradient centrifugation. In other embodiments, AAV is purified as described in US20020136710 using a solid support that includes a matrix to which an artificial receptor or receptor-like molecule that mediates AAV attachment is immobilized.

### Additional Elements in the Vectors

The vectors of the present disclosure can comprise promoters. In instances where the vector is a nucleic acid vector comprising nucleic acid encoding the capsid polypeptide, the promoter may facilitate expression of the nucleic acid encoding the capsid polypeptide. In instances where the vector is an AAV vector, the promoter may facilitate expression of a heterologous coding sequence, as described above.

In some examples, the promoters are AAV promoters, such as the p5, p19 or p40 promoter. In other examples, the promoters are derived from other sources. Examples of constitutive promoters include, without limitation, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, the human alpha 1-antitrypsin (hAAT) promoter and the EF1α promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Non-limiting examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system; the ecdysone insect promoter, the tetracycline-repressible system, the tetracycline-inducible system, the RU486-inducible system and the rapamycin-inducible system. Still other types of inducible promoters which may be useful in this context are those which are regulated by a specific physiological state, e.g., temperature, acute phase, a particular differentiation state of the cell, or in replicating cells only. In some embodiments, tissue specific promoters are used. Non-limiting examples of such promoters include the liver-specific thyroxin binding globulin (TBG) promoter, insulin promoter, glucagon promoter, somatostatin promoter, pancreatic polypeptide (PPY) promoter, synapsin-1 (Syn) promoter, creatine kinase (MCK) promoter, mammalian desmin (DES) promoter, a α-myosin heavy chain (a-MHC) promoter, a cardiac Troponin T (cTnT) promoter, beta-actin promoter, and hepatitis B virus core promoter. The selection of an appropriate promoter is well within the ability of one of ordinary skill in the art.

The vectors can also include transcriptional enhancers (e.g., an ApoE enhancer, translational signals, and transcriptional and translational termination signals. Examples of transcriptional termination signals include, but are not limited to, polyadenylation signal sequences, such as bovine growth hormone (BGH) poly(A), SV40 late poly(A), rabbit beta-globin (RBG) poly(A), thymidine kinase (TK) poly(A) sequences, and any variants thereof. In some embodiments, the transcriptional termination region is located downstream of the posttranscriptional regulatory element. In some embodiments, the transcriptional termination region is a polyadenylation signal sequence.

The vectors can include various posttranscriptional regulatory elements. In some embodiments, the posttranscriptional regulatory element can be a viral posttranscriptional regulatory element. Non-limiting examples of viral posttranscriptional regulatory element include woodchuck hepatitis virus posttranscriptional regulatory element (WPRE), hepatitis B virus posttranscriptional regulatory element (HBVPRE), RNA transport element, and any variants thereof. The RTE can be a rev response element (RRE), for example, a lentiviral RRE. A non-limiting example is bovine immunodeficiency virus rev response element (RRE). In some embodiments, the RTE is a constitutive transport element (CTE). Examples of CTE include, but are not limited to, Mason-Pfizer Monkey Virus CTE and Avian Leukemia Virus CTE.

A signal peptide sequence can also be included in the vector to provide for secretion of a polypeptide from a mammalian cell. Examples of signal peptides include, but are not limited to, the endogenous signal peptide for HGH and variants thereof; the endogenous signal peptide for interferons and variants thereof, including the signal peptide of type I, II and III interferons and variants thereof; and the endogenous signal peptides for known cytokines and variants thereof, such as the signal peptide of erythropoietin (EPO), insulin, TGF-β1, TNF, IL1-α, and IL1-β, and variants thereof. Typically, the nucleotide sequence of the signal peptide is located immediately upstream of the heterologous sequence (e.g., fused at the 5′ of the coding region of the protein of interest) in the vector.

In further examples, the vectors can contain a regulatory sequence that allows, for example, the translation of multiple proteins from a single mRNA. Non-limiting examples of such regulatory sequences include internal ribosome entry site (IRES) and 2A self-processing sequence, such as a 2A peptide site from foot-and-mouth disease virus (F2A sequence).

### Host Cells

Also provided herein are host cells comprising a nucleic acid molecule or vector or of the present disclosure. In some instances, the host cells are used to amplify, replicate, package and/or purify a polynucleotide or vector. In other examples, the host cells are used to express a heterologous sequence, such as one packaged within AAV vector. Exemplary host cells include prokaryotic and eukaryotic cells. In some instances, the host cell is a mammalian host cell. It is well within the skill of a skilled artisan to select an appropriate host cell for the expression, amplification, replication, packaging and/or purification of a polynucleotide, vector or rAAV virion of the present disclosure. Exemplary mammalian host cells include, but are not limited to, HEK293 cells, HeLa cells, Vero cells, HuH-7 cells, and HepG2 cells. In particular examples, the host cell is a hepatocyte or cell-line derived from a hepatocyte.

### Compositions

Also provided are compositions comprising the nucleic acid molecules, polypeptides and/or vectors of the present disclosure. In particular examples, provided are pharmaceutical compositions comprising the AAV vectors disclosed herein and a pharmaceutically acceptable carrier. The compositions can also comprise additional ingredients such as diluents, stabilizers, excipients, and adjuvants.

The carriers, diluents and adjuvants can include buffers such as phosphate, citrate, or other organic acids; antioxidants such as ascorbic acid; low molecular weight polypeptides (e.g., less than about 10 residues); proteins such as serum aAAVC.umin, gelatin or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). In some embodiments, the physiologically acceptable carrier is an aqueous pH buffered solution.

### Methods

The AAV vectors of the present disclosure, and compositions containing the AAV vectors, may be used in methods for the introduction of a heterologous coding sequence into a host cell. Such methods involve contacting the host cell with the AAV vector. This may be performed in vitro, ex vivo or in vivo. In particular embodiments, the host cell is a hepatocyte (e.g. a human hepatocyte).

When the methods are performed ex vivo or in vivo, typically the introduction of the heterologous sequence into the host cell is for therapeutic purposes, whereby expression of the heterologous sequence results in the treatment of a disease or condition. Thus, the AAV vectors disclosed herein can be administered to a subject (e.g., a human) in need thereof, such as subject with a disease or condition amendable to treatment with a protein, peptide or polynucleotide encoded by a heterologous sequence described herein.

When used in vivo, titers of AAV vectors to be administered to a subject will vary depending on, for example, the particular recombinant virus, the disease or disorder to be treated, the mode of administration, the treatment goal, the individual to be treated, and the cell type(s) being targeted, and can be determined by methods well known to those skilled in the art. Although the exact dosage will be determined on an individual basis, in most cases, typically, recombinant viruses of the present disclosure can be administered to a subject at a dose of between 1×10¹⁰ genome copies of the recombinant virus per kg of the subject and 1×10¹⁴ genome copies per kg. In other examples, less than 1×10¹⁰ genome copies may be sufficient for a therapeutic effect. In other examples, more than 1×10¹⁴ genome copies may be required for a therapeutic effect.

The route of the administration is not particularly limited. For example, a therapeutically effective amount of the AAV vector can be administered to the subject via, for example, intramuscular, intravaginal, intravenous, intraperitoneal, subcutaneous, epicutaneous, intradermal, rectal, intraocular, pulmonary, intracranial, intraosseous, oral, buccal, or nasal routes. The AAV vector can be administrated as a single dose or multiple doses, and at varying intervals.

Also provided are methods for producing an AAV vector described above and herein, i.e. one comprising a capsid polypeptide of the present disclosure. Such methods comprise culturing a host cell comprising a nucleic acid molecule encoding a capsid polypeptide the present disclosure, an AAV rep gene, a heterologous coding sequence flanked by AAV inverted terminal repeats, and helper functions for generating a productive AAV infection, under conditions suitable to facilitate assembly of an AAV vector comprising a capsid polypeptide of the present disclosure, wherein the capsid encapsidates the heterologous coding sequence.

In further aspects, provided are methods for enhancing the in vivo human hepatocyte transduction efficiency of an AAV vector. As demonstrated herein, some variable regions, and combinations of capsid variable regions, are important for efficient transduction of human hepatocytes by an AAV vector. For example, the VR-I from AAV2 and VR-VII and VR-VIII from AAV7 can impart efficient human hepatocyte vector uptake, while the VR-I from AAV8, the VR-IV from AAV10 and VR-VII from AAV7 can impart enhanced DNA to RNA conversion (i.e. efficient expression despite less efficient vector uptake).

Thus, provided herein are methods for producing a modified AAV vector that exhibits enhanced transgene expression in a human hepatocyte, where the methods include the steps of modifying the sequence of a reference capsid polypeptide at one or more of positions 263, 264, 265, 266, 267, 270, 274, 453, 458, 459, 462, 464, 468, 471, 472, 474, 475, 548, 549, 551, 552, 553, 554, 555, 556, 557, 558, 560, 561, 563 and 568, with numbering relative to SEQ ID NO:7, to thereby produce a modified capsid polypeptide that comprises a) amino acid residues N263, G264, T265, S266, G267, T270 and T274; b) amino acid residues S453, Q458, G459, Q462, L464, A468, A471, N472, S474 and A475; and c) amino acid residues T548, G549, T551, N552, K553, T554, T555, L556, E557, N558, L560, M561, N563, R568 and P569, with numbering relative to SEQ ID NO:7.

Methods for producing a modified AAV vector that exhibits enhanced transgene expression in a human hepatocyte also include those methods that include the steps of modifying the sequence of a reference capsid polypeptide at one or more of positions 263-274, 453-475 and 548-569 with numbering relative to SEQ ID NO:7, to thereby produce a modified capsid polypeptide that comprises: i) the sequence of amino acids NGTSGGATNDNT (SEQ ID NO:44) at positions 263-274, ii) the sequence of amino acids STGGTQGTQQLLFSQAGPANMSA (SEQ ID NO:36) at positions 453-475, and (iii) the sequence of amino acids TGATNKTTLENVLMTNEEEIRP (SEQ ID NO:33) at positions 548-569, with numbering relative to SEQ ID NO:7. In some examples, the modified capsid polypeptide that comprises i) the sequence of amino acids ISNGTSGGATNDNT (SEQ ID NO:46) at positions 261-274, ii) the sequence of amino acids QSTGGTQGTQQLLFSQAGPANMSA (SEQ ID NO:41) at positions 452-475, and (iii) the sequence of amino acids KTGATNKTTLENVLMTNEEEIRP (SEQ ID NO:39) at positions 547-569, with numbering relative to SEQ ID NO:7.

It will be understood that any modification or combination of modifications, e.g. amino acid replacement or substitution, amino acid deletion and/or amino acid insertion, will result in a change of amino acid sequence in the modified capsid polypeptide compared to the reference capsid polypeptide. Thus, for example, reference to modification does not include within its scope amino acid substitutions where one amino acid residue is substituted with the same amino acid residue, or modifications when an amino acid deletion is accompanied by an insertion of that deleted amino acid, such that there is no difference in the amino acid sequence of the modified capsid polypeptide compared to the reference capsid polypeptide sequence, i.e. the amino acid sequence of the modified capsid polypeptide can not be the same as (or must be different to) the amino acid sequence of the reference capsid polypeptide sequence.

Typically, the methods include an initial step of first identifying a reference capsid polypeptide for transducing human hepatocytes in vivo. The reference capsid polypeptide may be any AAV polypeptide, such as an AAV1, AAV2, AAV3, AAV3B, AAV4, AAV5, AAV6, AAV7, AAV8, AAV9, AAV10, AAV11, AAV12 or AAV13 capsid polypeptide, or a synthetic or chimeric capsid polypeptide. In illustrative embodiments, the reference polypeptide comprises at least or about 85%, 86%, 87%, 88%, 89%, 90%, 91%, 92%, 93%, 94%, 95%, 96%, 97%, 98%, or 99% sequence identity to the sequence set forth in SEQ ID NO:4. Reference capsid polypeptides include those comprising all or a portion of the VP1 protein, VP2 protein or VP3 protein. Thus, in some embodiments, the reference capsid polypeptide comprises all or a portion of a VP1 protein having at least or about 85%, 86%, 87%, 88%, 89%, 90%, 91%, 92%, 93%, 94%, 95%, 96%, 97%, 98%, or 99% sequence identity to the sequence set forth in SEQ ID NO:4 (also referred to as AAVC11.12); all or a portion of a VP2 protein having at least or about 85%, 86%, 87%, 88%, 89%, 90%, 91%, 92%, 93%, 94%, 95%, 96%, 97%, 98%, or 99% sequence identity to the VP2 protein set forth as amino acids 138-735 of SEQ ID NO:4; and all or a portion of a VP3 protein having at least or about 85%, 86%, 87%, 88%, 89%, 90%, 91%, 92%, 93%, 94%, 95%, 96%, 97%, 98%, or 99% sequence identity to the VP3 protein set forth as amino acids 204-735 of SEQ ID NO:4.

Methods for modifying the sequence of a reference capsid polypeptide or polynucleotide so as to produce a modified capsid polypeptide or polynucleotide are well known in the art, and any such method can be utilised so as to perform the methods of the present disclosure. For example, the modification of the sequence of the reference capsid polynucleotide to produce a modified capsid polynucleotide can be performed using any method known in the art, including recombinant and synthetic methods, performed (either in part or in whole) in silico and/or in vitro. In a particular example, the modification of the sequence is performed in silico, followed by de novo synthesis of the modified capsid polynucleotide having the modified sequence (e.g. by gene synthesis methods such as those involving the chemical synthesis of overlapping oligonucleotides following by gene assembly).

The modified capsid polynucleotides may be contained in nucleic acid vector, such as a plasmid, for subsequent expression, replication, amplification and/or manipulation. Vectors suitable for use in bacterial, insect and mammalian cells are widely described and well-known in the art. Those skilled in the art would appreciate that the vectors may also contain additional sequences and elements useful for the replication of the vector in prokaryotic and/or eukaryotic cells, selection of the vector and the expression of a heterologous sequence in a variety of host cells. For example, the vectors can include a prokaryotic replicon, which is a sequence having the ability to direct autonomous replication and maintenance of the vector extrachromosomally in a prokaryotic host cell, such as a bacterial host cell. Such replicons are well known in the art. In some embodiments, the vectors can include a shuttle element that makes the vectors suitable for replication and integration in both prokaryotes and eukaryotes. In addition, vectors may also include a gene whose expression confers a detectable marker such as a drug resistance gene, which allows for selection and maintenance of the host cells. Vectors may also have a reportable marker, such as gene encoding a fluorescent or other detectable protein. The nucleic acid vectors will likely also comprise other elements, including any one or more of those described below. Most typically, the vectors will comprise a promoter operably linked to the nucleic acid encoding the capsid protein.

The nucleic acid vectors can be constructed using known techniques, including, without limitation, the standard techniques of restriction endonuclease digestion, ligation, transformation, plasmid purification, in vitro or chemical synthesis of DNA, and DNA sequencing. The vectors comprising a modified capsid polynucleotide may be introduced into a host cell using any method known in the art.

Following modification, the modified capsid are then vectorised. Methods for vectorising a capsid polypeptide are well known in the art and non-limiting examples are described above.

The AAV vector produced by these methods typically has a level of transgene expression per cell that is enhanced compared to a reference AAV vector having a capsid comprising the reference capsid polypeptide. The level of transgene expression can be enhanced by at least or about, 10%, 20%, 30%, 40%, 50%, 60%, 70%, 80%, 90%, 100%, 200%, 300%, 400%, 500%, 600%, 700%, 800%, 900% 1000%, or more, e.g. the transgene expression of the AAV vector can be at least or about 2×, 3×, 4×, 5×, 6×, 7×, 8×, 9×, 10×, 12×, 13×, 14×, 15×, 16×, 17×, 18×, 19×, 20×, 30×, 40×, 50×, 60×, 70×, 80×, 90×, 100× or more compared to transgene expression from an unmodified AAV vector (i.e. an AAV vector comprising the reference capsid) in vivo. In some examples, this is assessed in an in vivo system that utilises human hepatocytes, such as a small animal (e.g. a mouse) with a chimeric liver comprising human hepatocytes (e.g. the humanised FRG mouse).

Thus, also provided are AAV vectors produced by the methods of the present disclosure.

In order that the invention may be readily understood and put into practical effect, particular preferred embodiments will now be described by way of the following non-limiting examples.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### EXAMPLES

### Example 1. Materials and Methods

### Adeno-Associated Virus Transgene Constructs

All vectors used in the study contain AAV2 ITR sequences. The AAV construct pLSP1-EGFP-WPRE-BGHpA, which encodes EGFP under the transcriptional control of a heterologous promoter containing one copy of the SERPINA1 (hAAT) promoter and two copies of the APOE enhancer element, has been previously reported (Dane et al., 2009, Mol Ther 17, 1548-1554) The barcoded versions of this construct include a 6-mer barcode between eGFP and WPRE (Cabanes-Creus, 2020, Sci Transl Med 12). The Venus and Cerulean versions were generated by substituting EGFP with either transgene encoding for the particular fluorophore.

DNA and RNA Isolation and cDNA Synthesis

Isolation of DNA and RNA and cDNA synthesis was performed as described in detail before (Cabanes-Creus, 2020, Sci Transl Med 12) without modifications. Briefly, DNA was extracted using a standard phenol:chloroform protocol and RNA with the Direct-Zol kit (Zymogen Cat #R2062).

### AAV Vector Packaging and Viral Production

AAV constructs were packaged into AAV capsids using HEK293 cells and a helper-virus-free system, as described previously (Xiao et al. 1998, J Virol 72, 2224-2232). Genomes were packaged in capsid variants using packaging plasmid constructs harbouring rep genes from AAV2 and a specific cap. Packaging of multiple barcoded ss-LSP1-EGFP-BC-WPRE-BGHpA at increasing concentrations was achieved as described recently (Cabanes-Creus et al., 2020, Mol Ther Methods Clin Dev 17, 1139-1154). All vectors were purified using iodixanol gradient ultracentrifugation, as previously described (Khan et al. 2011, Nat Protoc 6, 482-501). AAV preparations were tittered using droplet-digital PCR (see AAV titration section for details).

### Mouse Studies

All animal experimental procedures and care were approved by the joint Children's Medical Research Institute (CMRI) and The Children's Hospital at Westmead Animal Care and Ethics Committee. Fah^{-/-}Rag2^{-/-}Il2rg^{-/-} (FRG) mice (Azuma et al., 2007, Nat Biotechnol 25, 903-910) were bred, housed, engrafted, and monitored as recently described (Cabanes-Creus et al., 2020, Mol Ther Methods Clin Dev 17, 1139-1154). Levels of human cell engraftment were estimated by measuring the presence of human albumin in peripheral blood, using the human albumin ELISA quantitation kit (Bethyl Laboratories, catalogue no. E80-129). To evaluate the AAV transduction potential, mice were placed on 10% NTBC and were maintained in this condition until harvest. Mice were randomly assigned to experiments and transduced via intravenous injection (lateral tail vein) with the indicated vector doses. Mice were euthanised by CO2 inhalation either 1 or 2 weeks after transduction for barcoded NGS analysis or immunohistochemistry studies, respectively. To obtain murine and human single-cell suspensions from xenografted murine livers, the same collagenase perfusion procedure as recently described was followed (Cabanes-Creus et al., 2020, Sci Transl Med 12). For all experiments, cells were labelled with phycoerythrin (PE)-conjugated anti-human-HLA-ABC (clone W6/32, Invitrogen 12-9983-42; 1:20), biotin-conjugated anti-mouse-H-2Kb (clone AF6-88.5, BD Pharmigen 553568; 1:100), and allophycocyanin (APC)-conjugated-streptavidin (eBioscience 17-4317-82; 1:500). GFP-positive-labelled samples were sorted to a minimal 95% purity using a BD Influx Cell sorter. Flow cytometry was performed in the Flow Cytometry Facility, Westmead Institute for Medical Research (WIMR), Westmead, NSW, Australia. The data were analysed using FlowJo 7.6.1 (FlowJo LLC).

### Immunohistochemical Analysis of Mouse Livers

Immunohistochemistry was performed as described recently in detail (Cabanes-Creus et al. 2020, Mol Ther Methods Clin Dev 17, 1139-1154 (2020)) without modifications. Briefly, mouse livers were fixed with paraformaldehyde, cryo-protected in sucrose and froze in O.C.T (Tissue-Tek). Frozen liver sections (5 μm) were permeabilised in ice-cold methanol, then room temperature 0.1% Triton X-100, and then reacted with anti-human GAPDH antibody (Abcam, Cat #ab215227, Clone AF674), and DAPI (Invitrogen, D1306) at 0.08 ng/mL. Anti-Glutamine Synthetase (Abcam, Cat #ab73593) was used. After immunolabelling, the images were captured and analysed on a LSM800-Airyscan microscope using ZEN Black software.

### Barcode Amplification, NGS, and Distribution Analysis

150-base pair region surrounding the 6-mer barcode was amplified with Q5 High-Fidelity DNA Polymerase (NEB, catalog no. M0491L) using a barcode-specific forward primer (barcoded to allow multiplexing of different samples) and a common barcode reverse primer. NGS library preparations and sequencing using a 2×150 paired-end configurations were performed by Genewiz (Suzhou, China) using an Illumina MiSeq instrument. A workflow was written in Snakemake (5.6) (Koster et al., 2018, Bioinformatics 34, 3600) to process reads and count barcodes. Paired reads were merged using BBMerge and then filtered for reads of the expected length in a second pass through BBDuk, both from BBTools 38.68 (https://sourceforge.net/projects/bbmap/). The merged, filtered fastq files were passed to a Python (3.7) script that identified barcodes corresponding to AAV variants. NGS reads from the DNA and cDNA populations were normalized to the reads from the pre-injection mix.

Production of rAAV Crude Lysates

Production of rAAV crude lysates was performed as described before (Cabanes-Creus et al., 2019, Mol Ther Methods Clin Dev 12, 71-84) with a few modifications. The cell pellet was resuspended in 600 μL of Resuspension buffer (PBS, 10 mM Tris-HCl pH 8.5, 2 mM MgCl2) and subjected to three freeze-thaw cycles. Genomic and free plasmid DNA was removed by incubating with 200 U/mL at 37° C. for 1 hr of Benzonase (Merck KGaA, Cat #1.101695.0002; EMD Chemicals). 10% sodium deoxycholate was added to a final concentration of 0.5%, and subsequently added ¼ volume of 5M NaCl. The solution was then incubated at 37° C. for 30 minutes and cellular debris was removed by centrifugation for 30 min at 5,250×g at 4° C. The supernatant was then used to assess vector genome yield per 15-cm HEK293T plate.

### AAV Titration

AAV titration was performed via digital droplet PCR (ddPCR, Bio-Rad, Berkeley, CA, USA) using EvaGreen supermix (Bio-Rad, catalogue no. 1864034) and following the manufacturer's instructions. To detect AAV genomes on vectors, GFP primers were used. To detect the transgene encoding for Venus and Cerulean, respective matching primers were used.

### Example 2. Generation of Modified AAV Capsids Containing VR-I from AAV2, and VR-VII and VR-VIII from AAV7

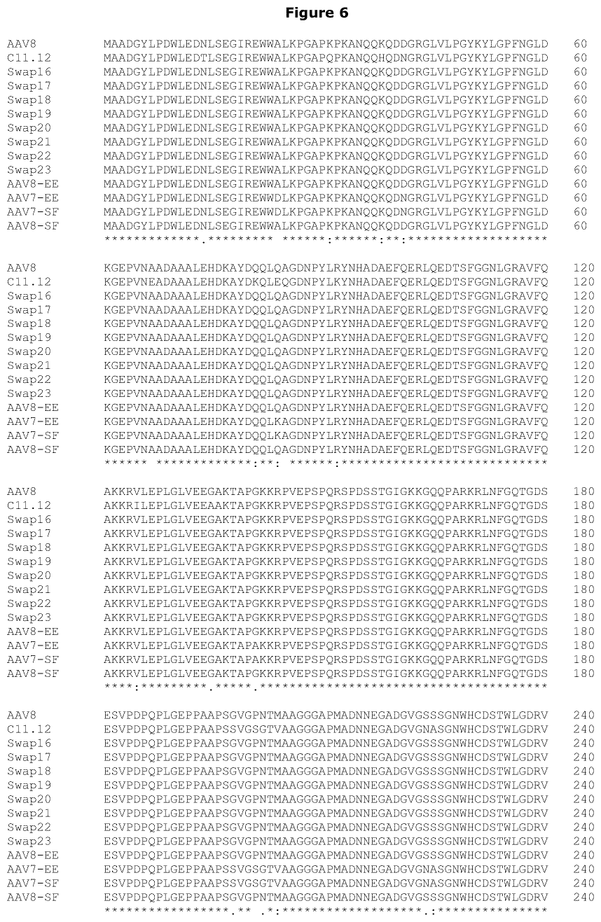

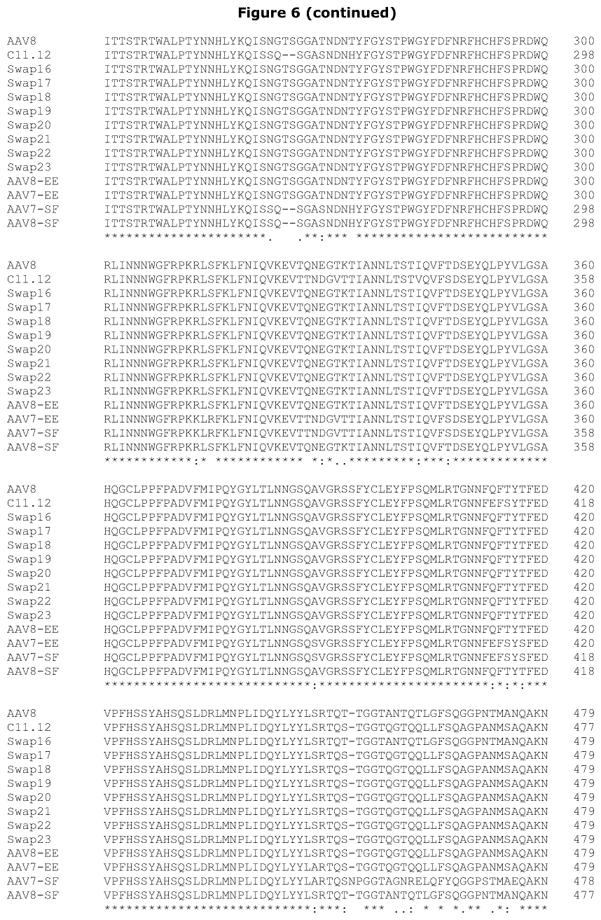

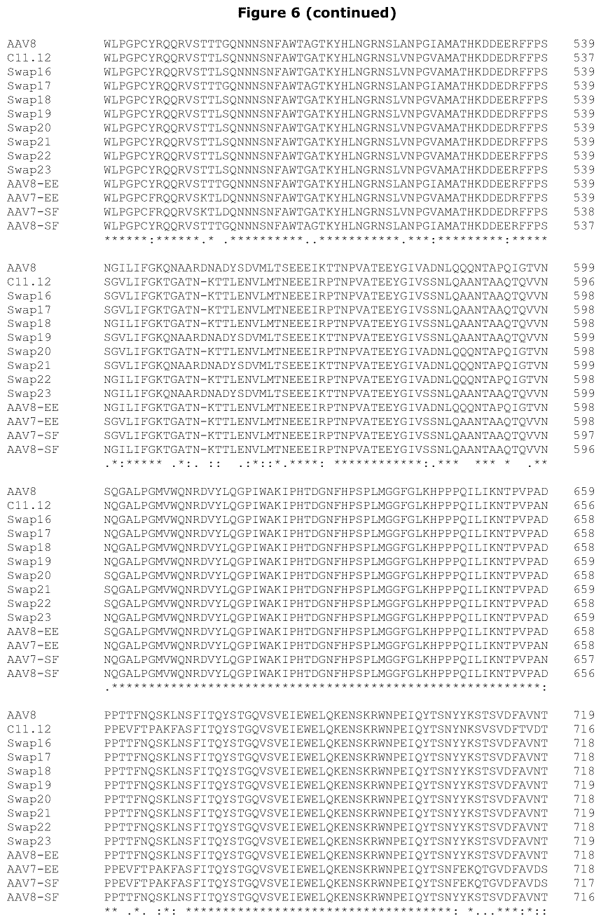

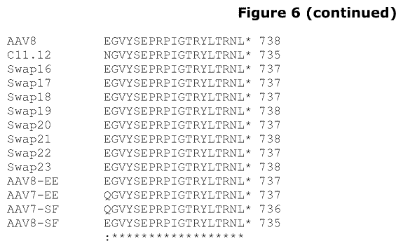

A previously-developed modified AAV, AAVC11.12, having a capsid sequence set forth in SEQ ID NO:4, exhibits superior transduction of human hepatocytes in vivo (International patent application number PCT/AU2021/050158). AAVC11.12 was developed using a shuffled DNA library, and upon analysis, it was observed that the capsid contained Variable Region (VR)-I from AAV2, VR-IV and VR-V from AAV10, and VR-VI to VR-VIII from AAV7. To help understand the difference in function between AAVC11.12 (SEQ ID NO:4) and AAV8 (SEQ ID NO:3), the latter of which does not transduce human hepatocytes efficiently, a series of domain swaps between the two AAV was generated (International patent application number PCT/AU2021/050158). In an extension of this work, a further modified AAV was produced in which the VR-I from AAV2, and VR-VII and VR-VIII from AAV7, was cloned into the AAV8 capsid (SEQ ID NO:3). The resulting capsid was named AAV8-SF and has the sequence set forth in SEQ ID NO:5. Table 3 below shows the specific amino acid changes between AAV8 and AAV8-SF (see also FIGS. 3 and 6).

TABLE 3
Amino acid changes between AAV8 and AAV8-SF

| Changes = 32 | AAV8 | AAV8-SF |
|---|---|---|
| 1 | N263 | del |
| 2 | G264 | del |
| 3 | T265 | S |
| 4 | S266 | Q |
| 5 | G267 | S |
| 6 | T270 | S |
| 7 | T274 | H |
| 8 | Q548 | T |
| 9 | N549 | G |
| 10 | A551 | T |
| 11 | R552 | del |
| 12 | D553 | N |
| 13 | N554 | K |
| 14 | A555 | T |
| 15 | D556 | T |
| 16 | Y557 | L |
| 17 | S558 | E |
| 18 | D559 | N |
| 19 | M561 | L |
| 20 | L562 | M |
| 21 | S564 | N |
| 22 | K569 | R |
| 23 | T570 | P |
| 24 | A583 | S |
| 25 | D584 | S |
| 26 | Q588 | A |
| 27 | Q589 | A |
| 28 | P593 | A |
| 29 | I595 | T |
| 30 | G596 | Q |
| 31 | T597 | V |
| 32 | S600 | N |

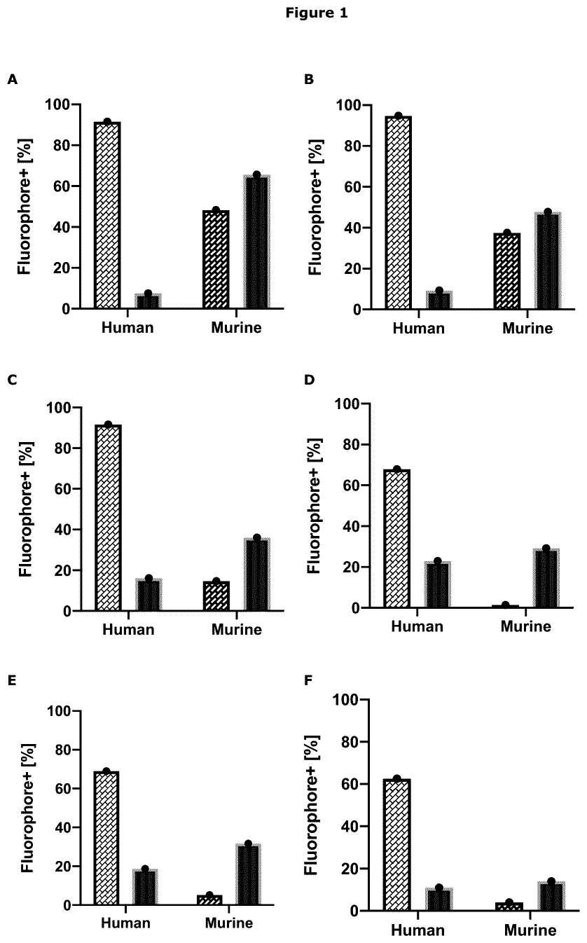

The performance of the AAV8-SF vector (AAV8-SF-Venus) was compared to that of parental AAV8 (AAV8-Cerulean) in a humanised FRG mouse with a replacement Index (RI) of 1.5%. The RI refers to the degree of endogenous hepatocyte replacement with xenotransplanted primary human hepatocytes within the liver of the host mouse, and can be estimated by measuring the human albumin levels in the blood (Sugahara et al. 2020, Semin Liver Dis 40, 189-212). As shown in FIG. 1B, the introduced changes resulted in significantly improved in vivo targeting of human hepatocytes by AAV8-SF when compared to parental AAV8. Given the hypothesised involvement of AAV7's VR-VII and VIII in enhanced human hepatocyte uptake of the vector in the context of competing murine hepatocytes, the inventors studied whether the incorporation of AAV2's VR-I in AAV7 would be sufficient to improve AAV7's performance in this model. Consequently, VR-I from AAV2 was cloned into the AAV7 capsid (SEQ ID NO:2) to generate AAV7-SF (SEQ ID NO:6) and the performance of AAV7-SF was compared to parental AAV7 in a humanised FRG mouse (RI=4.75%). As shown in FIG. 1C, AAV7-SF (AAV7-SF-Venus) presented marked human specificity when compared to the parental AAV7 (AAV7-Cerulean). Comparable studies using AAVC11.12 and AAV8 were also performed (FIG. 1A).

It was then investigated whether this observed phenotype would also be retained in mice presenting higher RI. The same Venus/Cerulean mixes were injected into three engrafted humanised FRG mice presenting an average RI of 84%, and similar results were observed (FIGS. 1D-F). As observed, AAVC11.12, AAV8-SF and AAV7-SF show superior human transduction than the respective controls. In all three cases, a reduction of Venus positive human hepatocytes (average of 92.65% in low; average of 66.5% in high RI) was observed. Interestingly, a reduction of Venus positive murine cells (average of 33.5% in low; average of 3.5% in high RI) was also observed, suggesting a marked preferential transduction of human over murine hepatocytes for AAVC11.12, AAV8-SF and AAV7-SF.

### Example 3. Generation of Additional Modified AAV Capsids

In order to further elucidate the effect of various capsid variable regions on the phenotype of an AAV vector, combinations of variables regions IV-V (AAV10 origin), and VI-VIII (AAV7 origin) from AAVC11.12 were systematically cloned into the AAV8 capsid scaffold to generate AAV8-Swap06 (also described in International patent application number PCT/AU2021/050158) and AAV8-Swap16-23 (shown schematically in FIG. 2). Specific amino acid changes between AAV8 and the swapped variants are shown in Table 4. FIG. 3 provides an alignment between AAVC11.12 (SEQ ID NO:4) and AAV8 (SEQ ID NO:3), also showing the residues from AAVC11.12 that were substituted into the AAV8 backbone. The amino acid and nucleic acid sequences of the resulting capsid polypeptides (i.e. Swap16-Swap23) are provided in Table 6.

TABLE 4
Amino acid changes between AAV8
and the Variable Region swaps.

| Changes = 44 | AAV8 | Swap6 |
|---|---|---|
| 1 | T453 | S |
| 2 | A458 | Q |
| 3 | N459 | G |
| 4 | T462 | Q |
| 5 | G464 | L |
| 6 | G468 | A |
| 7 | N471 | A |
| 8 | T472 | N |
| 9 | A474 | S |
| 10 | N475 | A |
| 11 | T495 | L |
| 12 | G496 | S |
| 13 | A507 | G |
| 14 | G508 | A |
| 15 | A520 | V |
| 16 | I524 | V |
| 17 | E534 | D |
| 18 | N540 | S |
| 19 | I542 | V |
| 20 | Q548 | T |
| 21 | N549 | G |
| 22 | A551 | T |
| 23 | R552 | del |
| 24 | D553 | N |
| 25 | N554 | K |
| 26 | A555 | T |
| 27 | D556 | T |
| 28 | Y557 | L |
| 29 | S558 | E |
| 30 | D559 | N |
| 31 | M561 | L |
| 32 | L562 | M |
| 33 | S564 | N |
| 34 | K569 | R |
| 35 | T570 | P |
| 36 | A583 | S |
| 37 | D584 | S |
| 38 | Q588 | A |
| 39 | Q589 | A |
| 40 | P593 | A |
| 41 | I595 | T |
| 42 | G596 | Q |
| 43 | T597 | V |
| 44 | S600 | N |
| Changes = 34 | AAV8 | Swap16 |
| 1 | T495 | L |
| 2 | G496 | S |
| 3 | A507 | G |
| 4 | G508 | A |
| 5 | A520 | V |
| 6 | I524 | V |
| 7 | E534 | D |
| 8 | N540 | S |
| 9 | I542 | V |
| 10 | Q548 | T |
| 11 | N549 | G |
| 12 | A551 | T |
| 13 | R552 | del |
| 14 | D553 | N |
| 15 | N554 | K |
| 16 | A555 | T |
| 17 | D556 | T |
| 18 | Y557 | L |
| 19 | S558 | E |
| 20 | D559 | N |
| 21 | M561 | L |
| 22 | L562 | M |
| 23 | S564 | N |
| 24 | K569 | R |
| 25 | T570 | P |
| 26 | A583 | S |
| 27 | D584 | S |
| 28 | Q588 | A |
| 29 | Q589 | A |
| 30 | P593 | A |
| 31 | I595 | T |
| 32 | G596 | Q |
| 33 | T597 | V |
| 34 | S600 | N |
| Changes = 38 | AAV8 | Swap17 |
| 1 | T453 | S |
| 2 | A458 | Q |
| 3 | N459 | G |
| 4 | T462 | Q |
| 5 | G464 | L |
| 6 | G468 | A |
| 7 | N471 | A |
| 8 | T472 | N |
| 9 | A474 | S |
| 10 | N475 | A |
| 11 | E534 | D |
| 12 | N540 | S |
| 13 | I542 | V |
| 14 | Q548 | T |
| 15 | N549 | G |
| 16 | A551 | T |
| 17 | R552 | del |
| 18 | D553 | N |
| 19 | N554 | K |
| 20 | A555 | T |
| 21 | D556 | T |
| 22 | Y557 | L |
| 23 | S558 | E |
| 24 | D559 | N |
| 25 | M561 | L |
| 26 | L562 | M |
| 27 | S564 | N |
| 28 | K569 | R |
| 29 | T570 | P |
| 30 | A583 | S |
| 31 | D584 | S |
| 32 | Q588 | A |
| 33 | Q589 | A |
| 34 | P593 | A |
| 35 | I595 | T |
| 36 | G596 | Q |
| 37 | T597 | V |
| 38 | S600 | N |
| Changes = 41 | AAV8 | Swap18 |
| 1 | T453 | S |
| 2 | A458 | Q |
| 3 | N459 | G |
| 4 | T462 | Q |
| 5 | G464 | L |
| 6 | G468 | A |
| 7 | N471 | A |
| 8 | T472 | N |
| 9 | A474 | S |
| 10 | N475 | A |
| 11 | T495 | L |
| 12 | G496 | S |
| 13 | A507 | G |
| 14 | G508 | A |
| 15 | A520 | V |
| 16 | I524 | V |
| 17 | Q548 | T |
| 18 | N549 | G |
| 19 | A551 | T |
| 20 | R552 | del |
| 21 | D553 | N |
| 22 | N554 | K |
| 23 | A555 | T |
| 24 | D556 | T |
| 25 | Y557 | L |
| 26 | S558 | E |
| 27 | D559 | N |
| 28 | M561 | L |
| 29 | L562 | M |
| 30 | S564 | N |
| 31 | K569 | R |
| 32 | T570 | P |
| 33 | A583 | S |
| 34 | D584 | S |
| 35 | Q588 | A |
| 36 | Q589 | A |
| 37 | P593 | A |
| 38 | I595 | T |
| 39 | G596 | Q |
| 40 | T597 | V |
| 41 | S600 | N |
| Changes = 28 | AAV8 | Swap19 |
| 1 | T453 | S |
| 2 | A458 | Q |
| 3 | N459 | G |
| 4 | T462 | Q |
| 5 | G464 | L |
| 6 | G468 | A |
| 7 | N471 | A |
| 8 | T472 | N |
| 9 | A474 | S |
| 10 | N475 | A |
| 11 | T495 | L |
| 12 | G496 | S |
| 13 | A507 | G |
| 14 | G508 | A |
| 15 | A520 | V |
| 16 | I524 | V |
| 17 | E534 | D |
| 18 | N540 | S |
| 19 | I542 | V |
| 20 | A583 | S |
| 21 | D584 | S |
| 22 | Q588 | A |
| 23 | Q589 | A |
| 24 | P593 | A |
| 25 | I595 | T |
| 26 | G596 | Q |
| 27 | T597 | V |
| 28 | S600 | N |
| Changes = 35 | AAV8 | Swap20 |
| 1 | T453 | S |
| 2 | A458 | Q |
| 3 | N459 | G |
| 4 | T462 | Q |
| 5 | G464 | L |
| 6 | G468 | A |
| 7 | N471 | A |
| 8 | T472 | N |
| 9 | A474 | S |
| 10 | N475 | A |
| 11 | T495 | L |
| 12 | G496 | S |
| 13 | A507 | G |
| 14 | G508 | A |
| 15 | A520 | V |
| 16 | I524 | V |
| 17 | E534 | D |
| 18 | N540 | S |
| 19 | I542 | V |
| 20 | Q548 | T |
| 21 | N549 | G |
| 22 | A551 | T |
| 23 | R552 | del |
| 24 | D553 | N |
| 25 | N554 | K |
| 26 | A555 | T |
| 27 | D556 | T |
| 28 | Y557 | L |
| 29 | S558 | E |
| 30 | D559 | N |
| 31 | M561 | L |
| 32 | L562 | M |
| 33 | S564 | N |
| 34 | K569 | R |
| 35 | T570 | P |
| Changes = 19 | AAV8 | Swap21 |
| 1 | T453 | S |
| 2 | A458 | Q |
| 3 | N459 | G |
| 4 | T462 | Q |
| 5 | G464 | L |
| 6 | G468 | A |
| 7 | N471 | A |
| 8 | T472 | N |
| 9 | A474 | S |
| 10 | N475 | A |
| 11 | T495 | L |
| 12 | G496 | S |
| 13 | A507 | G |
| 14 | G508 | A |
| 15 | A520 | V |
| 16 | I524 | V |
| 17 | E534 | D |
| 18 | N540 | S |
| 19 | I542 | V |
| Changes = 32 | AAV8 | Swap22 |
| 1 | T453 | S |
| 2 | A458 | Q |
| 3 | N459 | G |
| 4 | T462 | Q |
| 5 | G464 | L |
| 6 | G468 | A |
| 7 | N471 | A |
| 8 | T472 | N |
| 9 | A474 | S |
| 10 | N475 | A |
| 11 | T495 | L |
| 12 | G496 | S |
| 13 | A507 | G |
| 14 | G508 | A |
| 15 | A520 | V |
| 16 | I524 | V |
| 17 | Q548 | T |
| 18 | N549 | G |
| 19 | A551 | T |
| 20 | R552 | del |
| 21 | D553 | N |
| 22 | N554 | K |
| 23 | A555 | T |
| 24 | D556 | T |
| 25 | Y557 | L |
| 26 | S558 | E |
| 27 | D559 | N |
| 28 | M561 | L |
| 29 | L562 | M |
| 30 | S564 | N |
| 31 | K569 | R |
| 32 | T570 | P |
| Changes = 25 | AAV8 | Swap23 |
| 1 | T453 | S |
| 2 | A458 | Q |
| 3 | N459 | G |
| 4 | T462 | Q |
| 5 | G464 | L |
| 6 | G468 | A |
| 7 | N471 | A |
| 8 | T472 | N |
| 9 | A474 | S |
| 10 | N475 | A |
| 11 | T495 | L |
| 12 | G496 | S |
| 13 | A507 | G |
| 14 | G508 | A |
| 15 | A520 | V |
| 16 | I524 | V |
| 17 | A583 | S |
| 18 | D584 | S |
| 19 | Q588 | A |
| 20 | Q589 | A |
| 21 | P593 | A |
| 22 | I595 | T |
| 23 | G596 | Q |
| 24 | T597 | V |
| 25 | S600 | N |

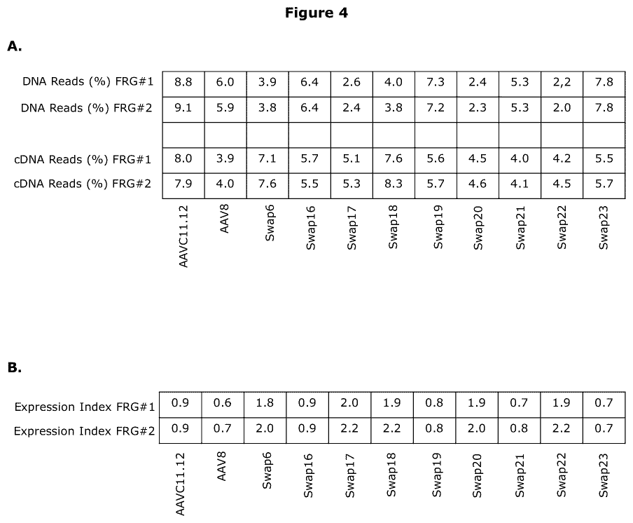

A barcoded vector mix that included control AAVC11.12, AAV8, AAV8-Swap6 and AAV8-Swap16-AAV8-Swap23 was generated and injected into two humanised FRG mice with high RI (average RI=89%) so as to facilitate comparison of the relative function of each vector. NGS results at the uptake (DNA) and functional (cDNA) levels were assessed and are shown in FIG. 4A. The expression index (ratio RNA reads:DNA reads) was also calculated and is shown in FIG. 4B. This is an indication of the vector's ability to drive transgene expression. It also likely reflects the timing of conversion from DNA to RNA. At 1 week post-injection, capsids that ‘express’ faster would represent a higher percentage of the total RNA reads, which would then result in a higher ‘expression index’. Comparing the performance of these AAV8-Swap16-Swap23 with that of AAV8-Swap6, the data suggested that, besides AAV8's VR-I, VR-IV (AAV10 origin) and VR-VII (AAV7 origin) were the main drivers of efficient transgene expression following human hepatocyte uptake. This can be inferred from the marked reduction in transgene expression of AAV8-Swap16 (reverted VR-IV) and the maintenance of this phenotype for AAV8-Swap22 (reverted VRs VI and VIII).

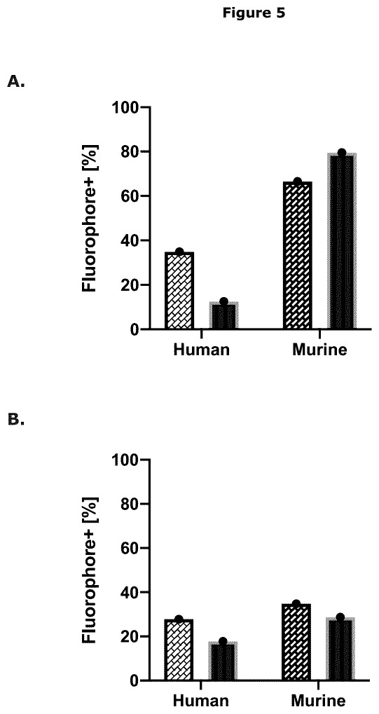

Parental AAV8 and AAV7 (SEQ ID NOs: 2 and 3, respectively) were then modified to contain the AAV8 VR-I, the AAV10 VR-IV and the AAV7 VR-VII, resulting in AAV8-EE and AAV7-EE, respectively (SEQ ID NOs: 7 and 8, respectively; see Table 5). Their performance was then assessed and compared to the parental AAV8 or parental AAV7 using a highly repopulated humanised FRG mouse with an RI of 97.5% and a vector dose of 3×10¹¹ vg per capsid. In both cases, an increase in human transduction was observed compared to the parental vector, although consistent with the previous observations both AAV8-EE and AAV7-EE retained their murine functional performance (FIG. 5).

### Example 4. Generation of AAV Capsids Having Further Modifications in the C11.12 Sequence

Three independent libraries using the C11.12 backbone were generated, based on naturally occurring amino acids in the VR-IV, VR-VII and VR-III in AAV-hu.Lvr02, AAV-hu.Lvr07, AAV7, AAV8 and AAV10. The libraries included amino acids at positions 446-461, 545-562 and 588-598, with numbering relative to C11.12 (SEQ ID NO:4) from AAV-hu.Lvr02, AAV-hu.Lvr07, AAV7, AAV8 and AAV10. The only exception made was at position Q450 (relative to C11.12), where only AAV-hu.Lvr06 harboured a different amino acid (N), which was not included in the library to minimize complexity. Insertions present in the serotypes were also included in the library. Amino acids that were conserved between these serotypes and C11.12 were not mutagenized.

The libraries were produced independently with PCR and Gibson Assembly. To do so, primers with degenerate codons at each of the mutagenized positions were used. For VR-IV and VR-VII, 3 extra primers were used to accommodate for the insertions at position 453 and 550, respectively. The libraries were built independently and cloned independently into the Functional Transduction Platform (harbouring the Liver-Specific Promoter (LSP)) and packaged, as described previously (Cabanes-Creus et al., 2020, Methods Clin Dev, 17:1139-1154). The libraries were then mixed and injected into two independent humanised FRG mice, one of which had received 5 mg of IVIg 24 h prior to library injection. After two weeks, the mice were super-infected with Adenovirus 5 to allow for replication in the human liver cells. Human hepatocytes with the replicated AAV were then harvested 4 days later. This process was repeated into three additional sets of humanised FRG mice in parallel, with one of them always receiving Intravenous immune globulin (IVIG) prior to injection, and the other always without IVIG.

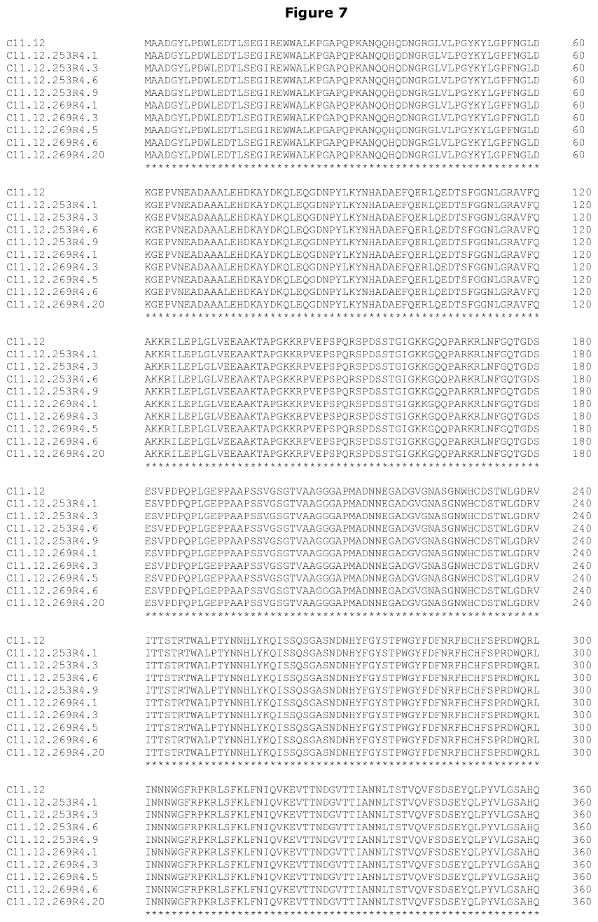

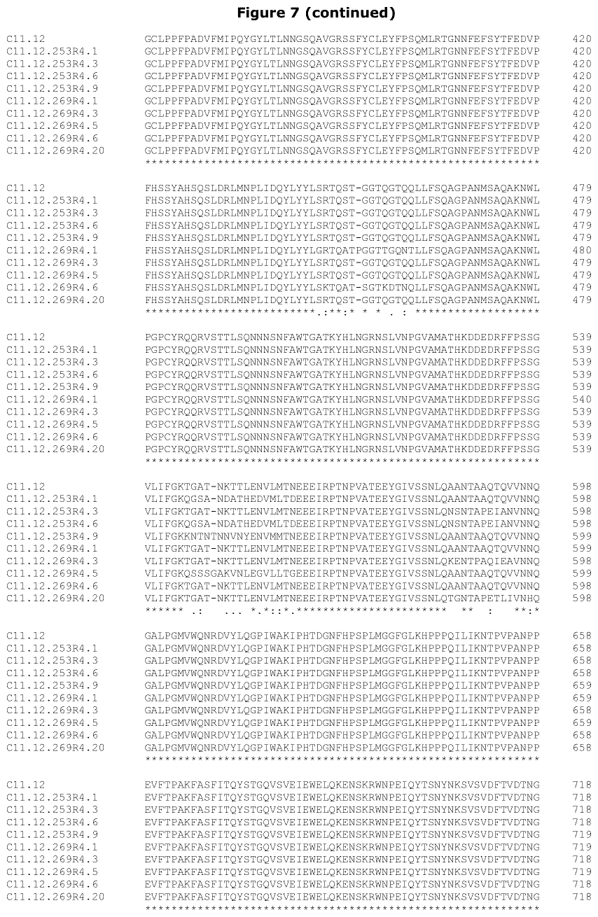

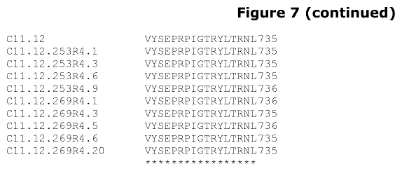

Variants were then isolated from the final (fourth) round of selection. Four variants were isolated from the fourth-round humanised FRG mouse that did not receive IVIG. These included C11.12.253R4.1 (amino acid sequence set forth in SEQ ID NO:47, nucleotide sequence set forth in SEQ ID NO: 56); C11.12.253R4.3 (amino acid sequence set forth in SEQ ID NO:48, nucleotide sequence set forth in SEQ ID NO: 57); C11.12.253R4.6 (amino acid sequence set forth in SEQ ID NO:49, nucleotide sequence set forth in SEQ ID NO: 58); and C11.12.253R4.9 (amino acid sequence set forth in SEQ ID NO:50, nucleotide sequence set forth in SEQ ID NO: 59). Five variants were isolated from the fourth-round humanised FRG mouse that did receive IVIG. These included C11.12.269R4.1 (amino acid sequence set forth in SEQ ID NO:51, nucleotide sequence set forth in SEQ ID NO: 60); C11.12.269R4.3 (amino acid sequence set forth in SEQ ID NO:52, nucleotide sequence set forth in SEQ ID NO: 61); C11.12.269R4.5 (amino acid sequence set forth in SEQ ID NO:53, nucleotide sequence set forth in SEQ ID NO: 62). C11.12.269R4.6 (amino acid sequence set forth in SEQ ID NO:54, nucleotide sequence set forth in SEQ ID NO: 63); and C11.12.269R4.20 (amino acid sequence set forth in SEQ ID NO:55, nucleotide sequence set forth in SEQ ID NO: 64). FIG. 7 provides an alignment of C11.12 and each of the variants.

C11.12.253R4.1 contains the following amino acid substitutions in the VR-VII, relative to C11.12: T546Q, A548S, T549A, K551D, T552A, L554H, N556D, L558M, M559L, and N561D.

C11.12.253R4.3 contains the following amino acid substitutions in the VR-VIII, relative to C11.12: A585N, A586S, A590P, Q591E, T592I, Q593A, V594N.

C11.12.253R4.6 contains the following amino acid substitutions in the VR-VII and VR-VIII, relative to C11.12: T546Q, A548S, T549A, K551D, T552A, L554H, N556D, L558M, M559L, N561D, A585N, A586S, A590P, Q591E, T592I, Q593A, V594N.

C11.12.253R4.9 contains the following amino acid substitutions in the VR-VII, relative to C11.12: T546K, G547N, A548T, T549N, T549_N550insT, K551N, T552V, T553N, L554Y and L558M.

C11.12.269R4.1 contains the following amino acid substitutions in the VR-IV, relative to C11.12: S447G, S451A, T452_G453insP, Q456T, T458Q, Q459N and Q460T.

C11.12.269R4.3 contains the following amino acid substitutions in the VR-VIII, relative to C11.12: A585K, A586E, A589P, T592I, Q593E and V594A.

C11.12.269R4.5 contains the following amino acid substitutions in the VR-VII, relative to C11.12: T546Q, G547S, A548S, T549S, T549_N550insG, N550A, T552V, T553N, N556G, M559L and N561G.

C11.12.269R4.6 contains the following amino acid substitutions in the VR-IV, relative to C11.12: R448K, S451A, G453S, Q456K, G457D and Q459N.

C11.12.269R4.20 contains the following amino acid substitutions in the VR-VIII, relative to C11.12: A585T, A586G, A590P, Q591E, Q593L, V594I and N597H.

In order to validate the above-described C11.12-derived variants, each (and C11.12) was packaged with 5 compatible barcoded transgenes encoding ITR2-LSP1-eGFP-Barcode-WPRE-ITR2, as shown in Table 5 below.

TABLE 5
Variant

| | |
|---|---|
| C11.12.253R4.1 | LSP-GFP-BC1_BC2_BC3_BC4_BC5 |
| C11.12.253R4.3 | LSP-GFP-BC6_BC7_BC8_BC9_BC10 |
| C11.12.253R4.6 | LSP-GFP-BC11_BC12_BC13_BC14_BC15 |
| C11.12.253R4.9 | LSP-GFP-BC16_BC17_BC18_BC19_BC20 |
| C11.12.269R4.1 | LSP-GFP-BC36_BC37_BC38_BC39_BC40 |
| C11.12.269R4.3 | LSP-GFP-BC21_BC22_BC23_BC24_BC25 |
| C11.12.269R4.5 | LSP-GFP-BC26_BC27_BC28_BC29_BC30 |
| C11.12.269R4.6 | LSP-GFP-BC31_BC32_BC33_BC34_BC35 |
| C11.12.269R4.20 | LSP-GFP-BC41_BC42_BC43_BC44_BC45 |
| C11.12 | LSP-GFP-BC46_BC47_BC48_BC49_BC50 |

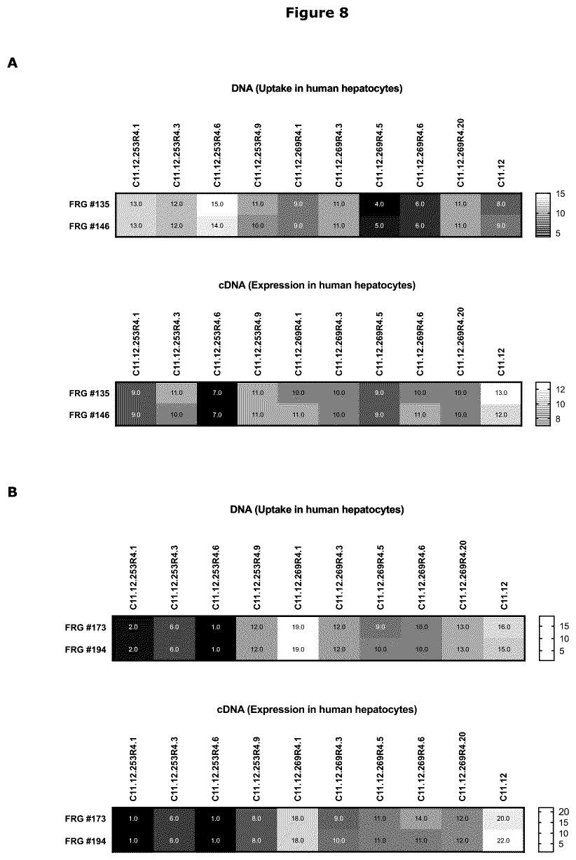

An equimolar vector mix of the 10 capsids was prepared, containing 5×10¹⁰ vector genomes per capsid (total of 5×10¹¹ vector genomes). One fifth of the mix (total of 1×10¹¹ vector genomes/mouse) was injected into four different humanized FRG mice, two of which were passively immunized with 5 mg of IVIg 24 hours pre-vector injection. The mg of albumin per mL of blood for the four FRG mice were 13.681 (FRG #135), 10.682 (FRG #146), 12.069 (FRG #173+IVIg) and 13.231 (FRG #194+IVIg), where FRG #'s correspond to those shown in FIG. 8. This corresponds to a replacement index (RI) of >80%. Three weeks after injection, the animals were perfused, the human hepatocytes isolated with FACS, DNA and RNA extracted, the barcoded region of the transgene from DNA and cDNA recovered with PCR, and the PCR amplicons sequenced by next-generation sequencing (NGS) in order to de-multiplex the barcode composition.

TABLE 6
Capsid Sequences

| SEQ | | |
|---|---|---|
| ID | | |
| NO | Name | Sequence |
| 1 | AAV2 | MAADGYLPDWLEDTLSEGIRQWWKLKPGPPPPKPAERHKDDSRGLVLPGYKYLGPFNGLD |
| | prototypic | KGEPVNEADAAALEHDKAYDRQLDSGDNPYLKYNHADAEFQERLKEDTSFGGNLGRAVFQ |
| | capsid-VP1 | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQRGNRQAATADVNTQGVLPG |
| | | MVWQDRDVYLQGPIWAKIPHTDGHFHPSPLMGGFGLKHPPPQILIKNTPVPANPSTTFSAA |
| | | KFASFITQYSTGQVSVEIEWELQKENSKRWNPEIQYTSNYNKSVNVDFTVDTNGVYSEPRP |
| | | IGTRYLTRNL |
| 2 | AAV7 | MAADGYLPDWLEDNLSEGIREWWDLKPGAPKPKANQQKQDNGRGLVLPGYKYLGPFNGL |
| | prototypic | DKGEPVNAADAAALEHDKAYDQQLKAGDNPYLRYNHADAEFQERLQEDTSFGGNLGRAVF |
| | capsid-VP1 | QAKKRVLEPLGLVEEGAKTAPAKKRPVEPSPQRSPDSSTGIGKKGQQPARKRLNFGQTGDS |
| | | ESVPDPQPLGEPPAAPSSVGSGTVAAGGGAPMADNNEGADGVGNASGNWHCDSTWLGD |
| | | RVITTSTRTWALPTYNNHLYKQISSETAGSTNDNTYFGYSTPWGYFDFNRFHCHFSPRDWQ |
| | | RLINNNWGFRPKKLRFKLFNIQVKEVTTNDGVTTIANNLTSTIQVFSDSEYQLPYVLGSAHQ |
| | | GCLPPFPADVFMIPQYGYLTLNNGSQSVGRSSFYCLEYFPSQMLRTGNNFEFSYSFEDVPFH |
| | | SSYAHSQSLDRLMNPLIDQYLYYLARTQSNPGGTAGNRELQFYQGGPSTMAEQAKNWLPG |
| | | PCFRQQRVSKTLDQNNNSNFAWTGATKYHLNGRNSLVNPGVAMATHKDDEDRFFPSSGV |
| | | LIFGKTGATNKTTLENVLMTNEEEIRPTNPVATEEYGIVSSNLQAANTAAQTQVVNNQGALP |
| | | GMVWQNRDVYLQGPIWAKIPHTDGNFHPSPLMGGFGLKHPPPQILIKNTPVPANPPEVFTP |
| | | AKFASFITQYSTGQVSVEIEWELQKENSKRWNPEIQYTSNFEKQTGVDFAVDSQGVYSEPR |
| | | PIGTRYLTRNL |
| 3 | AAV8 | MAADGYLPDWLEDNLSEGIREWWALKPGAPKPKANQQKQDDGRGLVLPGYKYLGPFNGL |
| | prototypic | DKGEPVNAADAAALEHDKAYDQQLQAGDNPYLRYNHADAEFQERLQEDTSFGGNLGRAVF |
| | capsid-VP1 | QAKKRVLEPLGLVEEGAKTAPGKKRPVEPSPQRSPDSSTGIGKKGQQPARKRLNFGQTGDS |
| | | ESVPDPQPLGEPPAAPSGVGPNTMAAGGGAPMADNNEGADGVGSSSGNWHCDSTWLGD |
| | | RVITTSTRTWALPTYNNHLYKQISNGTSGGATNDNTYFGYSTPWGYFDFNRFHCHFSPRD |
| | | WQRLINNNWGFRPKRLSFKLFNIQVKEVTQNEGTKTIANNLTSTIQVFTDSEYQLPYVLGSA |
| | | HQGCLPPFPADVFMIPQYGYLTLNNGSQAVGRSSFYCLEYFPSQMLRTGNNFQFTYTFEDVP |
| | | FHSSYAHSQSLDRLMNPLIDQYLYYLSRTQTTGGTANTQTLGFSQGGPNTMANQAKNWLP |
| | | GPCYRQQRVSTTTGQNNNSNFAWTAGTKYHLNGRNSLANPGIAMATHKDDEERFFPSNGI |
| | | LIFGKQNAARDNADYSDVMLTSEEEIKTTNPVATEEYGIVADNLQQQNTAPQIGTVNSQGA |
| | | LPGMVWQNRDVYLQGPIWAKIPHTDGNFHPSPLMGGFGLKHPPPQILIKNTPVPADPPTTF |
| | | NQSKLNSFITQYSTGQVSVEIEWELQKENSKRWNPEIQYTSNYYKSTSVDFAVNTEGVYSE |
| | | PRPIGTRYLTRNL |
| 4 | AAVC11.12 | MAADGYLPDWLEDTLSEGIREWWALKPGAPQPKANQQHQDNGRGLVLPGYKYLGPFNGL |
| | | DKGEPVNEADAAALEHDKAYDKQLEQGDNPYLKYNHADAEFQERLQEDTSFGGNLGRAVF |
| | | QAKKRILEPLGLVEEAAKTAPGKKRPVEPSPQRSPDSSTGIGKKGQQPARKRLNFGQTGDS |
| | | ESVPDPQPLGEPPAAPSSVGSGTVAAGGGAPMADNNEGADGVGNASGNWHCDSTWLGD |
| | | RVITTSTRTWALPTYNNHLYKQISSQSGASNDNHYFGYSTPWGYFDFNRFHCHFSPRDWQ |
| | | RLINNNWGFRPKRLSFKLFNIQVKEVTTNDGVTTIANNLTSTVQVFSDSEYQLPYVLGSAHQ |
| | | GCLPPFPADVFMIPQYGYLTLNNGSQAVGRSSFYCLEYFPSQMLRTGNNFEFSYTFEDVPFH |
| | | SSYAHSQSLDRLMNPLIDQYLYYLSRTQSTGGTQGTQQLLFSQAGPANMSAQAKNWLPGP |
| | | CYRQQRVSTTLSQNNNSNFAWTGATKYHLNGRNSLVNPGVAMATHKDDEDRFFPSSGVLI |
| | | FGKTGATNKTTLENVLMTNEEEIRPTNPVATEEYGIVSSNLQAANTAAQTQVVNNOGALPG |
| | | MVWQNRDVYLQGPIWAKIPHTDGNFHPSPLMGGFGLKHPPPQILIKNTPVPANPPEVFTPA |
| | | KFASFITQYSTGQVSVEIEWELQKENSKRWNPEIQYTSNYNKSVSVDFTVDTNGVYSEPRP |
| | | IGTRYLTRNL |
| 5 | AAV8-SF | MAADGYLPDWLEDNLSEGIREWWALKPGAPKPKANQQKQDDGRGLVLPGYKYLGPFNGL |
| | | DKGEPVNAADAAALEHDKAYDQQLQAGDNPYLRYNHADAEFQERLQEDTSFGGNLGRAVF |
| | | QAKKRVLEPLGLVEEGAKTAPGKKRPVEPSPQRSPDSSTGIGKKGQQPARKRLNFGQTGDS |
| | | ESVPDPQPLGEPPAAPSGVGPNTMAAGGGAPMADNNEGADGVGSSSGNWHCDSTWLGD |
| | | RVITTSTRTWALPTYNNHLYKQISSQSGASNDNHYFGYSTPWGYFDFNRFHCHFSPRDWQ |
| | | RLINNNWGFRPKRLSFKLFNIQVKEVTQNEGTKTIANNLTSTIQVFTDSEYQLPYVLGSAHQ |
| | | GCLPPFPADVFMIPQYGYLTLNNGSQAVGRSSFYCLEYFPSQMLRTGNNFQFTYTFEDVPFH |
| | | SSYAHSQSLDRLMNPLIDQYLYYLSRTQTTGGTANTQTLGFSQGGPNTMANQAKNWLPGP |
| | | CYRQQRVSTTTGQNNNSNFAWTAGTKYHLNGRNSLANPGIAMATHKDDEERFFPSNGILIF |
| | | GKTGATNKTTLENVLMTNEEEIRPTNPVATEEYGIVSSNLQAANTAAQTQVVNNQGALPGM |
| | | VWQNRDVYLQGPIWAKIPHTDGNFHPSPLMGGFGLKHPPPQILIKNTPVPADPPTTFNQSK |
| | | LNSFITQYSTGQVSVEIEWELQKENSKRWNPEIQYTSNYYKSTSVDFAVNTEGVYSEPRPIG |
| | | TRYLTRNL |
| 6 | AAV7-SF | MAADGYLPDWLEDNLSEGIREWWDLKPGAPKPKANQQKQDNGRGLVLPGYKYLGPFNGL |
| | | DKGEPVNAADAAALEHDKAYDQQLKAGDNPYLRYNHADAEFQERLQEDTSFGGNLGRAVF |
| | | QAKKRVLEPLGLVEEGAKTAPAKKRPVEPSPQRSPDSSTGIGKKGQQPARKRLNFGQTGDS |
| | | ESVPDPQPLGEPPAAPSSVGSGTVAAGGGAPMADNNEGADGVGNASGNWHCDSTWLGD |
| | | RVITTSTRTWALPTYNNHLYKQISSQSGASNDNHYFGYSTPWGYFDFNRFHCHFSPRDWQ |
| | | RLINNNWGFRPKKLRFKLFNIQVKEVTTNDGVTTIANNLTSTIQVFSDSEYQLPYVLGSAHQ |
| | | GCLPPFPADVFMIPQYGYLTLNNGSQSVGRSSFYCLEYFPSQMLRTGNNFEFSYSFEDVPFH |
| | | SSYAHSQSLDRLMNPLIDQYLYYLARTQSNPGGTAGNRELQFYQGGPSTMAEQAKNWLPG |
| | | PCFRQQRVSKTLDQNNNSNFAWTGATKYHLNGRNSLVNPGVAMATHKDDEDRFFPSSGV |
| | | LIFGKTGATNKTTLENVLMTNEEEIRPTNPVATEEYGIVSSNLQAANTAAQTQVVNNQGALP |
| | | GMVWQNRDVYLQGPIWAKIPHTDGNFHPSPLMGGFGLKHPPPQILIKNTPVPANPPEVFTP |
| | | AKFASFITQYSTGQVSVEIEWELQKENSKRWNPEIQYTSNFEKQTGVDFAVDSQGVYSEPR |
| | | PIGTRYLTRNL |
| 7 | AAV8-EE | MAADGYLPDWLEDNLSEGIREWWALKPGAPKPKANQQKQDDGRGLVLPGYKYLGPFNGL |
| | | DKGEPVNAADAAALEHDKAYDQQLQAGDNPYLRYNHADAEFQERLQEDTSFGGNLGRAVF |
| | | QAKKRVLEPLGLVEEGAKTAPGKKRPVEPSPQRSPDSSTGIGKKGQQPARKRLNFGQTGDS |
| | | ESVPDPQPLGEPPAAPSGVGPNTMAAGGGAPMADNNEGADGVGSSSGNWHCDSTWLGD |
| | | RVITTSTRTWALPTYNNHLYKQISNGTSGGATNDNTYFGYSTPWGYFDFNRFHCHFSPRD |
| | | WQRLINNNWGFRPKRLSFKLFNIQVKEVTQNEGTKTIANNLTSTIQVFTDSEYQLPYVLGSA |
| | | HQGCLPPFPADVFMIPQYGYLTLNNGSQAVGRSSFYCLEYFPSQMLRTGNNFQFTYTFEDVP |
| | | FHSSYAHSQSLDRLMNPLIDQYLYYLSRTQSTGGTQGTQQLLFSQAGPANMSAQAKNWLP |
| | | GPCYRQQRVSTTTGQNNNSNFAWTGATKYHLNGRNSLANPGIAMATHKDDEERFFPSNGI |
| | | LIFGKTGATNKTTLENVLMTNEEEIRPTNPVATEEYGIVADNLQQQNTAPQIGTVNSQGALP |
| | | GMVWQNRDVYLQGPIWAKIPHTDGNFHPSPLMGGFGLKHPPPQILIKNTPVPADPPTTFNQ |
| | | SKLNSFITQYSTGQVSVEIEWELQKENSKRWNPEIQYTSNYYKSTSVDFAVNTEGVYSEPR |
| | | PIGTRYLTRNL |
| 8 | AAV7-EE | MAADGYLPDWLEDNLSEGIREWWDLKPGAPKPKANQQKQDNGRGLVLPGYKYLGPFNGL |
| | | DKGEPVNAADAAALEHDKAYDQQLKAGDNPYLRYNHADAEFQERLQEDTSFGGNLGRAVF |
| | | QAKKRVLEPLGLVEEGAKTAPAKKRPVEPSPQRSPDSSTGIGKKGQQPARKRLNFGQTGDS |
| | | ESVPDPQPLGEPPAAPSSVGSGTVAAGGGAPMADNNEGADGVGNASGNWHCDSTWLGD |
| | | RVITTSTRTWALPTYNNHLYKQISNGTSGGATNDNTYFGYSTPWGYFDFNRFHCHFSPRD |
| | | WQRLINNNWGFRPKKLRFKLFNIQVKEVTTNDGVTTIANNLTSTIQVFSDSEYQLPYVLGSA |
| | | HQGCLPPFPADVFMIPQYGYLTLNNGSQSVGRSSFYCLEYFPSQMLRTGNNFEFSYSFEDVP |
| | | FHSSYAHSQSLDRLMNPLIDQYLYYLARTQSTGGTQGTQQLLFSQAGPANMSAQAKNWLP |
| | | GPCFRQQRVSKTLDQNNNSNFAWTGATKYHLNGRNSLVNPGVAMATHKDDEDRFFPSSG |
| | | VLIFGKTGATNKTTLENVLMTNEEEIRPTNPVATEEYGIVSSNLQAANTAAQTQVVNNQGAL |
| | | PGMVWQNRDVYLQGPIWAKIPHTDGNFHPSPLMGGFGLKHPPPQILIKNTPVPANPPEVFT |
| | | PAKFASFITQYSTGQVSVEIEWELQKENSKRWNPEIQYTSNFEKQTGVDFAVDSQGVYSEP |
| | | RPIGTRYLTRNL |
| 9 | AAV8 | MAADGYLPDWLEDNLSEGIREWWALKPGAPKPKANQQKQDDGRGLVLPGYKYLGPFNGL |
| | Swap16 | DKGEPVNAADAAALEHDKAYDQQLQAGDNPYLRYNHADAEFQERLQEDTSFGGNLGRAVF |
| | | QAKKRVLEPLGLVEEGAKTAPGKKRPVEPSPQRSPDSSTGIGKKGQQPARKRLNFGQTGDS |
| | | ESVPDPQPLGEPPAAPSGVGPNTMAAGGGAPMADNNEGADGVGSSSGNWHCDSTWLGD |
| | | RVITTSTRTWALPTYNNHLYKQISNGTSGGATNDNTYFGYSTPWGYFDFNRFHCHFSPRD |
| | | WQRLINNNWGFRPKRLSFKLFNIQVKEVTQNEGTKTIANNLTSTIQVFTDSEYQLPYVLGSA |
| | | HQGCLPPFPADVFMIPQYGYLTLNNGSQAVGRSSFYCLEYFPSQMLRTGNNFQFTYTFEDVP |
| | | FHSSYAHSQSLDRLMNPLIDQYLYYLSRTQTTGGTANTQTLGFSQGGPNTMANQAKNWLP |
| | | GPCYRQQRVSTTLSQNNNSNFAWTGATKYHLNGRNSLVNPGVAMATHKDDEDRFFPSSG |
| | | VLIFGKTGATNKTTLENVLMTNEEEIRPTNPVATEEYGIVSSNLQAANTAAQTQVVNNQGAL |
| | | PGMVWQNRDVYLQGPIWAKIPHTDGNFHPSPLMGGFGLKHPPPQILIKNTPVPADPPTTFN |
| | | QSKLNSFITQYSTGQVSVEIEWELQKENSKRWNPEIQYTSNYYKSTSVDFAVNTEGVYSEP |
| | | RPIGTRYLTRNL |
| 10 | AAV8 | MAADGYLPDWLEDNLSEGIREWWALKPGAPKPKANQQKQDDGRGLVLPGYKYLGPFNGL |
| | Swap17 | DKGEPVNAADAAALEHDKAYDQQLQAGDNPYLRYNHADAEFQERLQEDTSFGGNLGRAVF |
| | | QAKKRVLEPLGLVEEGAKTAPGKKRPVEPSPQRSPDSSTGIGKKGQQPARKRLNFGQTGDS |
| | | ESVPDPQPLGEPPAAPSGVGPNTMAAGGGAPMADNNEGADGVGSSSGNWHCDSTWLGD |
| | | RVITTSTRTWALPTYNNHLYKQISNGTSGGATNDNTYFGYSTPWGYFDFNRFHCHFSPRD |
| | | WQRLINNNWGFRPKRLSFKLFNIQVKEVTQNEGTKTIANNLTSTIQVFTDSEYQLPYVLGSA |
| | | HQGCLPPFPADVFMIPQYGYLTLNNGSQAVGRSSFYCLEYFPSQMLRTGNNFQFTYTFEDVP |
| | | FHSSYAHSQSLDRLMNPLIDQYLYYLSRTQSTGGTQGTQQLLFSQAGPANMSAQAKNWLP |
| | | GPCYRQQRVSTTTGQNNNSNFAWTAGTKYHLNGRNSLANPGIAMATHKDDEDRFFPSSG |
| | | VLIFGKTGATNKTTLENVLMTNEEEIRPTNPVATEEYGIVSSNLQAANTAAQTQVVNNQGAL |
| | | PGMVWQNRDVYLQGPIWAKIPHTDGNFHPSPLMGGFGLKHPPPQILIKNTPVPADPPTTFN |
| | | QSKLNSFITQYSTGQVSVEIEWELQKENSKRWNPEIQYTSNYYKSTSVDFAVNTEGVYSEP |
| | | RPIGTRYLTRNL |
| 11 | AAV8 | MAADGYLPDWLEDNLSEGIREWWALKPGAPKPKANQQKQDDGRGLVLPGYKYLGPFNGL |
| | Swap18 | DKGEPVNAADAAALEHDKAYDQQLQAGDNPYLRYNHADAEFQERLQEDTSFGGNLGRAVF |
| | | QAKKRVLEPLGLVEEGAKTAPGKKRPVEPSPQRSPDSSTGIGKKGQQPARKRLNFGQTGDS |
| | | ESVPDPQPLGEPPAAPSGVGPNTMAAGGGAPMADNNEGADGVGSSSGNWHCDSTWLGD |
| | | RVITTSTRTWALPTYNNHLYKQISNGTSGGATNDNTYFGYSTPWGYFDFNRFHCHFSPRD |
| | | WQRLINNNWGFRPKRLSFKLFNIQVKEVTQNEGTKTIANNLTSTIQVFTDSEYQLPYVLGSA |
| | | HQGCLPPFPADVFMIPQYGYLTLNNGSQAVGRSSFYCLEYFPSQMLRTGNNFQFTYTFEDVP |
| | | FHSSYAHSQSLDRLMNPLIDQYLYYLSRTQSTGGTQGTQQLLFSQAGPANMSAQAKNWLP |
| | | GPCYRQQRVSTTLSQNNNSNFAWTGATKYHLNGRNSLVNPGVAMATHKDDEERFFPSNGI |
| | | LIFGKTGATNKTTLENVLMTNEEEIRPTNPVATEEYGIVSSNLQAANTAAQTQVVNNQGALP |
| | | GMVWQNRDVYLQGPIWAKIPHTDGNFHPSPLMGGFGLKHPPPQILIKNTPVPADPPTTFNQ |
| | | SKLNSFITQYSTGQVSVEIEWELQKENSKRWNPEIQYTSNYYKSTSVDFAVNTEGVYSEPR |
| | | PIGTRYLTRNL |
| 12 | AAV8 | MAADGYLPDWLEDNLSEGIREWWALKPGAPKPKANQQKQDDGRGLVLPGYKYLGPFNGL |
| | Swap19 | DKGEPVNAADAAALEHDKAYDQQLQAGDNPYLRYNHADAEFQERLQEDTSFGGNLGRAVF |
| | | QAKKRVLEPLGLVEEGAKTAPGKKRPVEPSPQRSPDSSTGIGKKGQQPARKRLNFGQTGDS |
| | | ESVPDPQPLGEPPAAPSGVGPNTMAAGGGAPMADNNEGADGVGSSSGNWHCDSTWLGD |
| | | RVITTSTRTWALPTYNNHLYKQISNGTSGGATNDNTYFGYSTPWGYFDFNRFHCHFSPRD |
| | | WQRLINNNWGFRPKRLSFKLFNIQVKEVTQNEGTKTIANNLTSTIQVFTDSEYQLPYVLGSA |
| | | HQGCLPPFPADVFMIPQYGYLTLNNGSQAVGRSSFYCLEYFPSQMLRTGNNFQFTYTFEDVP |
| | | FHSSYAHSQSLDRLMNPLIDQYLYYLSRTQSTGGTQGTQQLLFSQAGPANMSAQAKNWLP |
| | | GPCYRQQRVSTTLSQNNNSNFAWTGATKYHLNGRNSLVNPGVAMATHKDDEDRFFPSSG |
| | | VLIFGKQNAARDNADYSDVMLTSEEEIKTTNPVATEEYGIVSSNLQAANTAAQTQVVNNQG |
| | | ALPGMVWQNRDVYLQGPIWAKIPHTDGNFHPSPLMGGFGLKHPPPQILIKNTPVPADPPTT |
| | | FNQSKLNSFITQYSTGQVSVEIEWELQKENSKRWNPEIQYTSNYYKSTSVDFAVNTEGVYS |
| | | EPRPIGTRYLTRNL |
| 13 | AAV8 | MAADGYLPDWLEDNLSEGIREWWALKPGAPKPKANQQKQDDGRGLVLPGYKYLGPFNGL |
| | Swap20 | DKGEPVNAADAAALEHDKAYDQQLQAGDNPYLRYNHADAEFQERLQEDTSFGGNLGRAVF |
| | | QAKKRVLEPLGLVEEGAKTAPGKKRPVEPSPQRSPDSSTGIGKKGQQPARKRLNFGQTGDS |
| | | ESVPDPQPLGEPPAAPSGVGPNTMAAGGGAPMADNNEGADGVGSSSGNWHCDSTWLGD |
| | | RVITTSTRTWALPTYNNHLYKQISNGTSGGATNDNTYFGYSTPWGYFDFNRFHCHFSPRD |
| | | WQRLINNNWGFRPKRLSFKLFNIQVKEVTQNEGTKTIANNLTSTIQVFTDSEYQLPYVLGSA |
| | | HQGCLPPFPADVFMIPQYGYLTLNNGSQAVGRSSFYCLEYFPSQMLRTGNNFQFTYTFEDVP |
| | | FHSSYAHSQSLDRLMNPLIDQYLYYLSRTQSTGGTQGTQQLLFSQAGPANMSAQAKNWLP |
| | | GPCYRQQRVSTTLSQNNNSNFAWTGATKYHLNGRNSLVNPGVAMATHKDDEDRFFPSSG |
| | | VLIFGKTGATNKTTLENVLMTNEEEIRPTNPVATEEYGIVADNLQQQNTAPQIGTVNSQGAL |
| | | PGMVWQNRDVYLQGPIWAKIPHTDGNFHPSPLMGGFGLKHPPPQILIKNTPVPADPPTTFN |
| | | QSKLNSFITQYSTGQVSVEIEWELQKENSKRWNPEIQYTSNYYKSTSVDFAVNTEGVYSEP |
| | | RPIGTRYLTRNL |
| 14 | AAV8 | MAADGYLPDWLEDNLSEGIREWWALKPGAPKPKANQQKQDDGRGLVLPGYKYLGPFNGL |
| | Swap21 | DKGEPVNAADAAALEHDKAYDQQLQAGDNPYLRYNHADAEFQERLQEDTSFGGNLGRAVF |
| | | QAKKRVLEPLGLVEEGAKTAPGKKRPVEPSPQRSPDSSTGIGKKGQQPARKRLNFGQTGDS |
| | | ESVPDPQPLGEPPAAPSGVGPNTMAAGGGAPMADNNEGADGVGSSSGNWHCDSTWLGD |
| | | RVITTSTRTWALPTYNNHLYKQISNGTSGGATNDNTYFGYSTPWGYFDFNRFHCHFSPRD |
| | | WQRLINNNWGFRPKRLSFKLFNIQVKEVTQNEGTKTIANNLTSTIQVFTDSEYQLPYVLGSA |
| | | HQGCLPPFPADVFMIPQYGYLTLNNGSQAVGRSSFYCLEYFPSQMLRTGNNFQFTYTFEDVP |
| | | FHSSYAHSQSLDRLMNPLIDQYLYYLSRTQSTGGTQGTQQLLFSQAGPANMSAQAKNWLP |
| | | GPCYRQQRVSTTLSQNNNSNFAWTGATKYHLNGRNSLVNPGVAMATHKDDEDRFFPSSG |
| | | VLIFGKQNAARDNADYSDVMLTSEEEIKTTNPVATEEYGIVADNLQQQNTAPQIGTVNSQG |
| | | ALPGMVWQNRDVYLQGPIWAKIPHTDGNFHPSPLMGGFGLKHPPPQILIKNTPVPADPPTT |
| | | FNQSKLNSFITQYSTGQVSVEIEWELQKENSKRWNPEIQYTSNYYKSTSVDFAVNTEGVYS |
| | | EPRPIGTRYLTRNL |
| 15 | AAV8 | MAADGYLPDWLEDNLSEGIREWWALKPGAPKPKANQQKQDDGRGLVLPGYKYLGPFNGL |
| | Swap22 | DKGEPVNAADAAALEHDKAYDQQLQAGDNPYLRYNHADAEFQERLQEDTSFGGNLGRAVF |
| | | QAKKRVLEPLGLVEEGAKTAPGKKRPVEPSPQRSPDSSTGIGKKGQQPARKRLNFGQTGDS |
| | | ESVPDPQPLGEPPAAPSGVGPNTMAAGGGAPMADNNEGADGVGSSSGNWHCDSTWLGD |
| | | RVITTSTRTWALPTYNNHLYKQISNGTSGGATNDNTYFGYSTPWGYFDFNRFHCHFSPRD |
| | | WQRLINNNWGFRPKRLSFKLFNIQVKEVTQNEGTKTIANNLTSTIQVFTDSEYQLPYVLGSA |
| | | HQGCLPPFPADVFMIPQYGYLTLNNGSQAVGRSSFYCLEYFPSQMLRTGNNFQFTYTFEDVP |
| | | FHSSYAHSQSLDRLMNPLIDQYLYYLSRTQSTGGTQGTQQLLFSQAGPANMSAQAKNWLP |
| | | GPCYRQQRVSTTLSQNNNSNFAWTGATKYHLNGRNSLVNPGVAMATHKDDEERFFPSNGI |
| | | LIFGKTGATNKTTLENVLMTNEEEIRPTNPVATEEYGIVADNLQQQNTAPQIGTVNSQGALP |
| | | GMVWQNRDVYLQGPIWAKIPHTDGNFHPSPLMGGFGLKHPPPQILIKNTPVPADPPTTFNQ |
| | | SKLNSFITQYSTGQVSVEIEWELQKENSKRWNPEIQYTSNYYKSTSVDFAVNTEGVYSEPR |
| | | PIGTRYLTRNL |
| 16 | AAV8 | MAADGYLPDWLEDNLSEGIREWWALKPGAPKPKANQQKQDDGRGLVLPGYKYLGPFNGL |
| | Swap23 | DKGEPVNAADAAALEHDKAYDQQLQAGDNPYLRYNHADAEFQERLQEDTSFGGNLGRAVF |
| | | QAKKRVLEPLGLVEEGAKTAPGKKRPVEPSPQRSPDSSTGIGKKGQQPARKRLNFGQTGDS |
| | | ESVPDPQPLGEPPAAPSGVGPNTMAAGGGAPMADNNEGADGVGSSSGNWHCDSTWLGD |
| | | RVITTSTRTWALPTYNNHLYKQISNGTSGGATNDNTYFGYSTPWGYFDFNRFHCHFSPRD |
| | | WQRLINNNWGFRPKRLSFKLFNIQVKEVTQNEGTKTIANNLTSTIQVFTDSEYQLPYVLGSA |
| | | HQGCLPPFPADVFMIPQYGYLTLNNGSQAVGRSSFYCLEYFPSQMLRTGNNFQFTYTFEDVP |
| | | FHSSYAHSQSLDRLMNPLIDQYLYYLSRTQSTGGTQGTQQLLFSQAGPANMSAQAKNWLP |
| | | GPCYRQQRVSTTLSQNNNSNFAWTGATKYHLNGRNSLVNPGVAMATHKDDEERFFPSNGI |
| | | LIFGKQNAARDNADYSDVMLTSEEEIKTTNPVATEEYGIVSSNLQAANTAAQTQVVNNQGA |
| | | LPGMVWQNRDVYLQGPIWAKIPHTDGNFHPSPLMGGFGLKHPPPQILIKNTPVPADPPTTF |
| | | NQSKLNSFITQYSTGQVSVEIEWELQKENSKRWNPEIQYTSNYYKSTSVDFAVNTEGVYSE |
| | | PRPIGTRYLTRNL |
| 17 | AAV7 (nt) | atggctgccgatggttatcttccagattggctcgaggacaacctctctgagggcattcgcgagtggtgggacctgaa |
| | | acctggagccccgaaacccaaagccaaccagcaaaagcaggacaacggccggggtctggtgcttcctggctaca |
| | | agtacctcggacccttcaacggactcgacaagggggagcccgtcaacgcggcggacgcagcggccctcgagcac |
| | | gacaaggcctacgaccagcagctcaaagcgggtgacaatccgtacctgcggtataaccacgccgacgccgagttt |
| | | caggagcgtctgcaagaagatacgtcatttgggggcaacctcgggcgagcagtcttccaggccaagaagcgggt |
| | | tctcgaacctctcggtctggttgaggaaggcgctaagacggctcctgcaaagaagagaccggtagagccgtcacc |
| | | tcagcgttcccccgactcctccacgggcatcggcaagaaaggccagcagcccgccagaaagagactcaatttcgg |
| | | tcagactggcgactcagagtcagtccccgaccctcaacctctcggagaacctccagcagcgccctctagtgtggga |
| | | tctggtacagtggctgcaggcggtggcgcaccaatggcagacaataacgaaggtgccgacggagtgggtaatgc |
| | | ctcaggaaattggcattgcgattccacatggctgggcgacagagtcattaccaccagcacccgaacctgggccctg |
| | | cccacctacaacaaccacctctacaagcaaatctccagtgaaactgcaggtagtaccaacgacaacacctacttcg |
| | | gctacagcaccccctgggggtattttgactttaacagattccactgccacttctcaccacgtgactggcagcgactca |
| | | tcaacaacaactggggattccggcccaagaagctgcggttcaagctcttcaacatccaggtcaaggaggtcacga |
| | | cgaatgacggcgttacgaccatcgctaataaccttaccagcacgattcaggtattctcggactcggaataccagctg |
| | | ccgtacgtcctcggctctgcgcaccagggctgcctgcctccgttcccggcggacgtcttcatgattcctcagtacggc |
| | | tacctgactctcaacaatggcagtcagtctgtgggacgttcctccttctactgcctggagtacttcccctctcagatgct |
| | | gagaacgggcaacaactttgagttcagctacagcttcgaggacgtgcctttccacagcagctacgcacacagcca |
| | | gagcctggaccggctgatgaatcccctcatcgaccagtacttgtactacctggccagaacacagagtaacccagga |
| | | ggcacagctggcaatcgggaactgcagttttaccagggcgggccttcaactatggccgaacaagccaagaattgg |
| | | ttacctggaccttgcttccggcaacaaagagtctccaaaacgctggatcaaaacaacaacagcaactttgcttgga |
| | | ctggtgccaccaaatatcacctgaacggcagaaactcgttggttaatcccggcgtcgccatggcaactcacaagga |
| | | cgacgaggaccgctttttcccatccagcggagtcctgatttttggaaaaactggagcaactaacaaaactacattgg |
| | | aaaatgtgttaatgacaaatgaagaagaaattcgtcctactaatcctgtagccacggaagaatacgggatagtca |
| | | gcagcaacttacaagcggctaatactgcagcccagacacaagttgtcaacaaccagggagccttacctggcatgg |
| | | tctggcagaaccgggacgtgtacctgcagggtcccatctgggccaagattcctcacacggatggcaactttcaccc |
| | | gtctcctttgatgggcggctttggacttaaacatccgcctcctcagatcctgatcaagaacactcccgttcccgctaat |
| | | cctccggaggtgtttactcctgccaagtttgcttcgttcatcacacagtacagcaccggacaagtcagcgtggaaat |
| | | cgagtgggagctgcagaaggaaaacagcaagcgctggaacccggagattcagtacacctccaactttgaaaagc |
| | | agactggtgtggactttgccgttgacagccagggtgtttactctgagcctcgccctattggcactcgttacctcacccg |
| | | taatctg |
| 18 | AAV8 (nt) | atggctgccgatggttatcttccagattggctcgaggacaacctctctgagggcattcgcgagtggtgggcgctgaa |
| | | acctggagccccgaagcccaaagccaaccagcaaaagcaggacgacggccggggtctagtgcttcctggctaca |
| | | agtacctcggacccttcaacggactcgacaagggggagcccgtcaacgcggcggacgcagcggccctcgagcac |
| | | gacaaggcctacgaccagcagctgcaggcgggtgacaatccgtacctgcggtataaccacgccgacgccgagttt |
| | | caggagcgtctgcaagaagatacgtcttttgggggcaacctcgggcgagcagtcttccaggccaagaagcgggtt |
| | | ctcgaacctctcggtctggttgaggaaggcgctaagacggctcctggaaagaagagaccggtagagccatcaccc |
| | | cagcgttctccagactcctctacgggcatcggcaagaaaggccaacagcccgccagaaaaagactcaattttggtc |
| | | agactggcgactcagagtcagttccagaccctcaacctctcggagaacctccagcagcgccctctggtgtgggacc |
| | | taatacaatggctgcaggcggtggcgcaccaatggcagacaataacgaaggcgccgacggagtgggtagttcct |
| | | cgggaaattggcattgcgattccacatggctgggcgacagagtcatcaccaccagcacccgaacctgggccctgc |
| | | ccacctacaacaaccacctctacaagcaaatctccaacgggacatcgggaggagccaccaacgacaacacctact |
| | | tcggctacagcaccccctgggggtattttgactttaacagattccactgccacttttcaccacgtgactggcagcgac |
| | | tcatcaacaacaactggggattccggcccaagagactcagcttcaagctcttcaacatccaggtcaaggaggtcac |
| | | gcagaatgaaggcaccaagaccatcgccaataacctcaccagcaccatccaggtgtttacggactcggagtacca |
| | | gctgccgtacgttctcggctctgcccaccagggctgcctgcctccgttcccggcggacgtgttcatgattccccagta |
| | | cggctacctaacactcaacaacggtagtcaggccgtgggacgctcctccttctactgcctggaatactttccttcgca |
| | | gatgctgagaaccggcaacaacttccagtttacttacaccttcgaggacgtgcctttccacagcagctacgcccaca |
| | | gccagagcttggaccggctgatgaatcctctgattgaccagtacctgtactacttgtctcggactcaaacaacagga |
| | | ggcacggcaaatacgcagactctgggcttcagccaaggtgggcctaatacaatggccaatcaggcaaagaactg |
| | | gctgccaggaccctgttaccgccaacaacgcgtctcaacgacaaccgggcaaaacaacaatagcaactttgcctg |
| | | gactgctgggaccaaataccatctgaatggaagaaattcattggctaatcctggcatcgctatggcaacacacaaa |
| | | gacgacgaggagcgtttttttcccagtaacgggatcctgatttttggcaaacaaaatgctgccagagacaatgcgg |
| | | attacagcgatgtcatgctcaccagcgaggaagaaatcaaaaccactaaccctgtggctacagaggaatacggta |
| | | tcgtggcagataacttgcagcagcaaaacacggctcctcaaattggaactgtcaacagccagggggccttacccg |
| | | gtatggtctggcagaaccgggacgtgtacctgcagggtcccatctgggccaagattcctcacacggacggcaactt |
| | | ccacccgtctccgctgatgggcggctttggcctgaaacatcctccgcctcagatcctgatcaagaacacgcctgtac |
| | | ctgcggatcctccgaccaccttcaaccagtcaaagctgaactctttcatcacgcaatacagcaccggacaggtcagc |
| | | gtggaaattgaatgggagctgcagaaggaaaacagcaagcgctggaaccccgagatccagtacacctccaacta |
| | | ctacaaatctacaagtgtggactttgctgttaatacagaaggcgtgtactctgaaccccgccccattggcacccgtta |
| | | cctcacccgtaatctgtaa |
| 19 | AAVC11.12 | atggctgccgatggttatcttccagattggctcgaggacactctctctgaaggcattcgcgagtggtgggcgctgaa |
| | (nt) | acctggagctccacaacccaaggccaaccaacagcatcaggacaacggcaggggtcttgtgcttcctgggtacaa |
| | | gtacctcggacccttcaacggactcgacaagggagagccggtcaacgaggcagacgccgcggccctcgagcacg |
| | | acaaggcctacgacaagcagctcgagcagggggacaacccgtacctcaagtacaaccacgccgacgccgagttt |
| | | caggagcgtcttcaagaagatacgtcttttgggggcaaccttggcagagcagtcttccaggccaaaaagaggatc |
| | | cttgagcctcttggtctggttgaggaagctgctaagacggctcctggaaagaagagaccggtagaaccgtcacctc |
| | | agcgttcccccgactcctccacgggcatcggcaagaaaggccagcagcccgccagaaagagactcaatttcggtc |
| | | agactggcgactcagagtcagtccccgaccctcaacctctcggagaacctccagcagcgccctctagtgtgggatc |
| | | tggtacagtggctgcaggcggtggcgcaccaatggcagacaataacgaaggtgccgacggagtgggtaatgcct |
| | | caggaaattggcattgcgattccacatggctgggcgacagagtcatcaccaccagcaccagaacctgggccctgc |
| | | ccacttacaacaaccatctctacaagcaaatctccagccaatcaggagcttcaaacgacaaccactacttcggctac |
| | | agcaccccctgggggtattttgactttaacagattccactgccacttttcaccacgtgactggcagcgactcatcaac |
| | | aacaactggggattccggcccaagagactcagcttcaagctcttcaacatccaagtcaaggaggtcacgacgaat |
| | | gatggcgtcacgaccatcgctaataaccttaccagcacggttcaagtcttctcggactcggagtaccagcttccgta |
| | | cgtcctcggctctgcgcaccagggctgcctccctccgttcccggcggacgtgttcatgattccgcagtacggctacct |
| | | aacgctcaacaatggcagccaggcagtgggacggtcatccttttactgcctggaatattttccatctcaaatgctgcg |
| | | aactggaaacaattttgaattcagctacaccttcgaggacgtgcctttccacagcagctacgcacacagccagagct |
| | | tggaccgactgatgaatcctctcattgaccagtacctgtactacttatccagaactcagtccacaggaggaactcaa |
| | | ggtacccagcaattgttattttctcaagctgggcctgcaaacatgtcggctcaggccaagaactggctgcctggacc |
| | | ttgctaccggcagcagcgagtctccacgacactgtcgcaaaacaacaacagcaactttgcttggactggtgccacc |
| | | aaatatcacctgaacggcagaaactcgttggttaatcccggcgtcgccatggcaactcacaaggacgacgaggac |
| | | cgctttttcccatccagcggagtcctgatttttggaaaaactggagcaactaacaaaactacattggaaaatgtgtta |
| | | atgacaaatgaagaagaaattcgtcctactaatcctgtagccacggaagaatacgggatagtcagcagcaactta |
| | | caagcggctaatactgcagcccagacacaagttgtcaacaaccagggagccttacctggcatggtctggcagaac |
| | | cgggacgtgtacctgcagggtcccatctgggccaagattcctcacacggatggcaactttcacccgtctcctttgatg |
| | | ggcggctttggacttaaacatccgcctcctcagatcctgatcaagaacactcccgttcccgctaatcctccggaggtg |
| | | tttactcctgccaagtttgcttcgttcatcacacagtacagcaccggacaagtcagcgtggaaatcgagtgggagct |
| | | gcagaaggaaaacagcaagcgctggaacccggagattcagtacacttcaaactacaacaagtctgttagtgtgg |
| | | actttactgtagacactaatggcgtgtattcagagcctcgccccattggcaccagatacctgactcgtaatctgtaa |
| 20 | AAV8-SF | atggctgccgatggttatcttccagattggctcgaggacaacctctctgagggcattcgcgagtggtgggcgctgaa |
| | (nt) | acctggagccccgaagcccaaagccaaccagcaaaagcaggacgacggccggggtctagtgcttcctggctaca |
| | | agtacctcggacccttcaacggactcgacaagggggagcccgtcaacgcggcggacgcagcggccctcgagcac |
| | | gacaaggcctacgaccagcagctgcaggcgggtgacaatccgtacctgcggtataaccacgccgacgccgagttt |
| | | caggagcgtctgcaagaagatacgtcttttgggggcaacctcgggcgagcagtcttccaggccaagaagcgggtt |
| | | ctcgaacctctcggtctggttgaggaaggcgctaagacggctcctggaaagaagagaccggtagagccatcaccc |
| | | cagcgttctccagactcctctacgggcatcggcaagaaaggccaacagcccgccagaaaaagactcaattttggtc |
| | | agactggcgactcagagtcagttccagaccctcaacctctcggagaacctccagcagcgccctctggtgtgggacc |
| | | taatacaatggctgcaggcggtggcgcaccaatggcagacaataacgaaggcgccgacggagtgggtagttcct |
| | | cgggaaattggcattgcgattccacatggctgggcgacagagtcatcaccaccagcaccagaacctgggccctgc |
| | | ccacttacaacaaccatctctacaagcaaatctccagccaatcaggagcttcaaacgacaaccactacttcggctac |
| | | agcaccccctgggggtattttgactttaacagattccactgccacttttcaccacgtgactggcagcgactcatcaac |
| | | aacaactggggattccggcccaagagactcagcttcaagctcttcaacatccaggtcaaggaggtcacgcagaat |
| | | gaaggcaccaagaccatcgccaataacctcaccagcaccatccaggtgtttacggactcggagtaccagctgccgt |
| | | acgttctcggctctgcccaccagggctgcctgcctccgttcccggcggacgtgttcatgattccccagtacggctacc |
| | | taacactcaacaacggtagtcaggccgtgggacgctcctccttctactgcctggaatactttccttcgcagatgctga |
| | | gaaccggcaacaacttccagtttacttacaccttcgaggacgtgcctttccacagcagctacgcccacagccagagc |
| | | ttggaccggctgatgaatcctctgattgaccagtacctgtactacttgtctcggactcaaacaacaggaggcacggc |
| | | aaatacgcagactctgggcttcagccaaggtgggcctaatacaatggccaatcaggcaaagaactggctgccag |
| | | gaccctgttaccgccaacaacgcgtctcaacgacaaccgggcaaaacaacaatagcaactttgcctggactgctg |
| | | ggaccaaataccatctgaatggaagaaattcattggctaatcctggcatcgctatggcaacacacaaggacgacg |
| | | aggagcgctttttcccatccaacggaatcctgatttttggaaaaactggagcaactaacaaaactacattggaaaat |
| | | gtgttaatgacaaatgaagaagaaattcgtcctactaatcctgtagccacggaagaatacgggatagtcagcagc |
| | | aacttacaagcggctaatactgcagcccagacacaagttgtcaacaaccagggagccttacctggcatggtctggc |
| | | agaaccgggacgtgtacctgcagggtcccatctgggccaagattcctcacacggacggcaacttccacccgtctcc |
| | | gctgatgggcggctttggcctgaaacatcctccgcctcagatcctgatcaagaacacgcctgtacctgcggatcctc |
| | | cgaccaccttcaaccagtcaaagctgaactctttcatcacgcaatacagcaccggacaggtcagcgtggaaattga |
| | | atgggagctgcagaaggaaaacagcaagcgctggaaccccgagatccagtacacctccaactactacaaatcta |
| | | caagtgtggactttgctgttaatacagaaggcgtgtactctgaaccccgccccattggcacccgttacctcacccgta |
| | | atctgtaa |
| 21 | AAV7-SF | atggctgccgatggttatcttccagattggctcgaggacaacctctctgagggcattcgcgagtggtgggacctgaa |
| | (nt) | acctggagccccgaaacccaaagccaaccagcaaaagcaggacaacggccggggtctggtgcttcctggctaca |
| | | agtacctcggacccttcaacggactcgacaagggggagcccgtcaacgcggcggacgcagcggccctcgagcac |
| | | gacaaggcctacgaccagcagctcaaagcgggtgacaatccgtacctgcggtataaccacgccgacgccgagttt |
| | | caggagcgtctgcaagaagatacgtcatttgggggcaacctcgggcgagcagtcttccaggccaagaagcgggt |
| | | tctcgaacctctcggtctggttgaggaaggcgctaagacggctcctgcaaagaagagaccggtagagccgtcacc |
| | | tcagcgttcccccgactcctccacgggcatcggcaagaaaggccagcagcccgccagaaagagactcaatttcgg |
| | | tcagactggcgactcagagtcagtccccgaccctcaacctctcggagaacctccagcagcgccctctagtgtggga |
| | | tctggtacagtggctgcaggcggtggcgcaccaatggcagacaataacgaaggtgccgacggagtgggtaatgc |
| | | ctcaggaaattggcattgcgattccacatggctgggcgacagagtcatcaccaccagcaccagaacctgggccct |
| | | gcccacttacaacaaccatctctacaagcaaatctccagccaatcaggagcttcaaacgacaaccactacttcggct |
| | | acagcaccccctgggggtattttgactttaacagattccactgccacttctcaccacgtgactggcagcgactcatca |
| | | acaacaactggggattccggcccaagaagctgcggttcaagctcttcaacatccaggtcaaggaggtcacgacga |
| | | atgacggcgttacgaccatcgctaataaccttaccagcacgattcaggtattctcggactcggaataccagctgccg |
| | | tacgtcctcggctctgcgcaccagggctgcctgcctccgttcccggcggacgtcttcatgattcctcagtacggctac |
| | | ctgactctcaacaatggcagtcagtctgtgggacgttcctccttctactgcctggagtacttcccctctcagatgctga |
| | | gaacgggcaacaactttgagttcagctacagcttcgaggacgtgcctttccacagcagctacgcacacagccaga |
| | | gcctggaccggctgatgaatcccctcatcgaccagtacttgtactacctggccagaacacagagtaacccaggagg |
| | | cacagctggcaatcgggaactgcagttttaccaggggggccttcaactatggccgaacaagccaagaattggtt |
| | | acctggaccttgcttccggcaacaaagagtctccaaaacgctggatcaaaacaacaacagcaactttgcttggact |
| | | ggtgccaccaaatatcacctgaacggcagaaactcgttggttaatcccggcgtcgccatggcaactcacaaggac |
| | | gacgaggaccgctttttcccatccagcggagtcctgatttttggaaaaactggagcaactaacaaaactacattgga |
| | | aaatgtgttaatgacaaatgaagaagaaattcgtcctactaatcctgtagccacggaagaatacgggatagtcagc |
| | | agcaacttacaagcggctaatactgcagcccagacacaagttgtcaacaaccagggagccttacctggcatggtct |
| | | ggcagaaccgggacgtgtacctgcagggtcccatctgggccaagattcctcacacggatggcaactttcacccgtc |
| | | tcctttgatgggcggctttggacttaaacatccgcctcctcagatcctgatcaagaacactcccgttcccgctaatcct |
| | | ccggaggtgtttactcctgccaagtttgcttcgttcatcacacagtacagcaccggacaagtcagcgtggaaatcga |
| | | gtgggagctgcagaaggaaaacagcaagcgctggaacccggagattcagtacacctccaactttgaaaagcag |
| | | actggtgtggactttgccgttgacagccagggtgtttactctgagcctcgccctattggcactcgttacctcacccgta |
| | | atctgtaa |
| 22 | AAV8-EE | atggctgccgatggttatcttccagattggctcgaggacaacctctctgagggcattcgcgagtggtgggcgctgaa |
| | (nt) | acctggagccccgaagcccaaagccaaccagcaaaagcaggacgacggccggggtctagtgcttcctggctaca |
| | | agtacctcggacccttcaacggactcgacaagggggagcccgtcaacgcggcggacgcagcggccctcgagcac |
| | | gacaaggcctacgaccagcagctgcaggcgggtgacaatccgtacctgcggtataaccacgccgacgccgagttt |
| | | caggagcgtctgcaagaagatacgtcttttgggggcaacctcgggcgagcagtcttccaggccaagaagcgggtt |
| | | ctcgaacctctcggtctggttgaggaaggcgctaagacggctcctggaaagaagagaccggtagagccatcaccc |
| | | cagcgttctccagactcctctacgggcatcggcaagaaaggccaacagcccgccagaaaaagactcaattttggtc |
| | | agactggcgactcagagtcagttccagaccctcaacctctcggagaacctccagcagcgccctctggtgtgggacc |
| | | taatacaatggctgcaggcggtggcgcaccaatggcagacaataacgaaggcgccgacggagtgggtagttcct |
| | | cgggaaattggcattgcgattccacatggctgggcgacagagtcatcaccaccagcaccagaacctgggccctgc |
| | | ccacttacaacaaccatctctacaagcaaatctccagccaatcaggagcttcaaacgacaaccactacttcggctac |
| | | agcaccccctgggggtattttgactttaacagattccactgccacttttcaccacgtgactggcagcgactcatcaac |
| | | aacaactggggattccggcccaagagactcagcttcaagctcttcaacatccaggtcaaggaggtcacgcagaat |
| | | gaaggcaccaagaccatcgccaataacctcaccagcaccatccaggtgtttacggactcggagtaccagctgccgt |
| | | acgttctcggctctgcccaccagggctgcctgcctccgttcccggcggacgtgttcatgattccccagtacggctacc |
| | | taacactcaacaacggtagtcaggccgtgggacgctcctccttctactgcctggaatactttccttcgcagatgctga |
| | | gaaccggcaacaacttccagtttacttacaccttcgaggacgtgcctttccacagcagctacgcccacagccagagc |
| | | ttggaccggctgatgaatcctctgattgaccagtacctgtactacttatccagaactcagtccacaggaggaactca |
| | | aggtacccagcaattgttattttctcaagctgggcctgcaaacatgtcggctcaggccaagaactggctgcctggac |
| | | cttgctaccggcagcagcgagtctccacgacaacggggcaaaacaacaacagcaactttgcttggactggtgcca |
| | | ccaaatatcacctgaacggcagaaactcgttggctaatcccggcatcgccatggcaacacacaaggacgacgag |
| | | gagcgtttttttcccagtaacgggatcctgatttttggcaaaactggtgccacaaacaaaacgactttggagaatgtc |
| | | ttgatgaccaacgaggaagaaatcagacccactaaccctgtggctacagaggaatacggtatcgtggcagataac |
| | | ttgcagcagcaaaacacggctcctcaaattggaactgtcaacagccagggggccttacccggtatggtctggcag |
| | | aaccgggacgtgtacctgcagggtcccatctgggccaagattcctcacacggacggcaacttccacccgtctccgct |
| | | gatgggcggctttggcctgaaacatcctccgcctcagatcctgatcaagaacacgcctgtacctgcggatcctccga |
| | | ccaccttcaaccagtcaaagctgaactctttcatcacgcaatacagcaccggacaggtcagcgtggaaattgaatg |
| | | ggagctgcagaaggaaaacagcaagcgctggaaccccgagatccagtacacctccaactactacaaatctacaa |
| | | gtgtggactttgctgttaatacagaaggcgtgtactctgaaccccgccccattggcacccgttacctcacccgtaatc |
| | | tgtaa |
| 23 | AAV7-EE | atggctgccgatggttatcttccagattggctcgaggacaacctctctgagggcattcgcgagtggtgggacctgaa |
| | (nt) | acctggagccccgaaacccaaagccaaccagcaaaagcaggacaacggccggggtctggtgcttcctggctaca |
| | | agtacctcggacccttcaacggactcgacaagggggagcccgtcaacgcggcggacgcagcggccctcgagcac |
| | | gacaaggcctacgaccagcagctcaaagcgggtgacaatccgtacctgcggtataaccacgccgacgccgagttt |
| | | caggagcgtctgcaagaagatacgtcatttgggggcaacctcgggcgagcagtcttccaggccaagaagcgggt |
| | | tctcgaacctctcggtctggttgaggaaggcgctaagacggctcctgcaaagaagagaccggtagagccgtcacc |
| | | tcagcgttcccccgactcctccacgggcatcggcaagaaaggccagcagcccgccagaaagagactcaatttcgg |
| | | tcagactggcgactcagagtcagtccccgaccctcaacctctcggagaacctccagcagcgccctctagtgtggga |
| | | tctggtacagtggctgcaggcggtggcgcaccaatggcagacaataacgaaggtgccgacggagtgggtaatgc |
| | | ctcaggaaattggcattgcgattccacatggctgggcgacagagtcatcaccaccagcacccgaacctgggccctg |
| | | cccacctacaacaaccacctctacaagcaaatctccaacgggacatcgggaggagccaccaacgacaacacctac |
| | | ttcggctacagcaccccctgggggtattttgactttaacagattccactgccacttctcaccacgtgactggcagcga |
| | | ctcatcaacaacaactggggattccggcccaagaagctgcggttcaagctcttcaacatccaggtcaaggaggtca |
| | | cgacgaatgacggcgttacgaccatcgctaataaccttaccagcacgattcaggtattctcggactcggaatacca |
| | | gctgccgtacgtcctcggctctgcgcaccagggctgcctgcctccgttcccggcggacgtcttcatgattcctcagta |
| | | cggctacctgactctcaacaatggcagtcagtctgtgggacgttcctccttctactgcctggagtacttcccctctcag |
| | | atgctgagaacgggcaacaactttgagttcagctacagcttcgaggacgtgcctttccacagcagctacgcacaca |
| | | gccagagcctggaccggctgatgaatcccctcatcgaccagtacttgtactacctggccagaactcagtccacagg |
| | | aggaactcaaggtacccagcaattgttattttctcaagctgggcctgcaaacatgtcggctcaggccaagaactgg |
| | | ctgcctggaccttgcttccggcaacaaagagtctccaaaacgctggatcaaaacaacaacagcaactttgcttgga |
| | | ctggtgccaccaaatatcacctgaacggcagaaactcgttggttaatcccggcgtcgccatggcaactcacaagga |
| | | cgacgaggaccgctttttcccatccagcggagtcctgatttttggaaaaactggagcaactaacaaaactacattgg |
| | | aaaatgtgttaatgacaaatgaagaagaaattcgtcctactaatcctgtagccacggaagaatacgggatagtca |
| | | gcagcaacttacaagcggctaatactgcagcccagacacaagttgtcaacaaccagggagccttacctggcatgg |
| | | tctggcagaaccgggacgtgtacctgcagggtcccatctgggccaagattcctcacacggatggcaactttcaccc |
| | | gtctcctttgatgggcggctttggacttaaacatccgcctcctcagatcctgatcaagaacactcccgttcccgctaat |
| | | cctccggaggtgtttactcctgccaagtttgcttcgttcatcacacagtacagcaccggacaagtcagcgtggaaat |
| | | cgagtgggagctgcagaaggaaaacagcaagcgctggaacccggagattcagtacacctccaactttgaaaagc |
| | | agactggtgtggactttgccgttgacagccagggtgtttactctgagcctcgccctattggcactcgttacctcacccg |
| | | taatctgtaa |
| 24 | AAV8 | atggctgccgatggttatcttccagattggctcgaggacaacctctctgagggcattcgcgagtggtgggcgctgaa |
| | Swap16 | acctggagccccgaagcccaaagccaaccagcaaaagcaggacgacggccggggtctagtgcttcctggctaca |
| | (nt) | agtacctcggacccttcaacggactcgacaagggggagcccgtcaacgcggcggacgcagcggccctcgagcac |
| | | gacaaggcctacgaccagcagctgcaggcgggtgacaatccgtacctgcggtataaccacgccgacgccgagttt |
| | | caggagcgtctgcaagaagatacgtcttttgggggcaacctcgggcgagcagtcttccaggccaagaagcgggtt |
| | | ctcgaacctctcggtctggttgaggaaggcgctaagacggctcctggaaagaagagaccggtagagccatcaccc |
| | | cagcgttctccagactcctctacgggcatcggcaagaaaggccaacagcccgccagaaaaagactcaattttggtc |
| | | agactggcgactcagagtcagttccagaccctcaacctctcggagaacctccagcagcgccctctggtgtgggacc |
| | | taatacaatggctgcaggcggtggcgcaccaatggcagacaataacgaaggcgccgacggagtgggtagttcct |
| | | cgggaaattggcattgcgattccacatggctgggcgacagagtcatcaccaccagcaccagaacctgggccctgc |
| | | ccacctacaacaaccacctctacaagcaaatctccaacgggacatcgggaggagccaccaacgacaacacctact |
| | | tcggctacagcaccccctgggggtattttgactttaacagattccactgccacttttcaccacgtgactggcagcgac |
| | | tcatcaacaacaactggggattccggcccaagagactcagcttcaagctcttcaacatccaggtcaaggaggtcac |
| | | gcagaatgaaggcaccaagaccatcgccaataacctcaccagcaccatccaggtgtttacggactcggagtacca |
| | | gctgccgtacgttctcggctctgcccaccagggctgcctgcctccgttcccggcggacgtgttcatgattccccagta |
| | | cggctacctaacactcaacaacggtagtcaggccgtgggacgctcctccttctactgcctggaatactttccttcgca |
| | | gatgctgagaaccggcaacaacttccagtttacttacaccttcgaggacgtgcctttccacagcagctacgcccaca |
| | | gccagagcttggaccggctgatgaatcctctgattgaccagtacctgtactacttatccagaactcagaccacagga |
| | | ggaactgcaaatacccagacattgggattttctcaaggtgggcctaacaccatggcgaatcaggccaagaactgg |
| | | ctgcctggaccttgctaccggcagcagcgagtctccacgacactgtcgcaaaacaacaacagcaactttgcttgga |
| | | ctggtgccaccaaatatcacctgaacggcagaaactcgttggttaatcccggcgtcgccatggcaacacacaagg |
| | | acgacgaggaccgctttttcccatccagcggagtcctgatttttggaaaaactggagcaactaacaaaactacattg |
| | | gaaaatgtgttaatgacaaatgaagaagaaattcgtcctactaatcctgtagccacggaagaatacgggatagtc |
| | | agcagcaacttacaagcggctaatactgcagcccagacacaagttgtcaacaaccagggagccttacctggcatg |
| | | gtctggcagaaccgggacgtgtacctgcagggtcccatctgggccaagattcctcacacggacggcaacttccacc |
| | | cgtctccgctgatgggcggctttggcctgaaacatcctccgcctcagatcctgatcaagaacacgcctgtacctgcg |
| | | gatcctccgaccaccttcaaccagtcaaagctgaactctttcatcacgcaatacagcaccggacaggtcagcgtgg |
| | | aaattgaatgggagctgcagaaggaaaacagcaagcgctggaaccccgagatccagtacacctccaactactac |
| | | aaatctacaagtgtggactttgctgttaatacagaaggcgtgtactctgaaccccgccccattggcacccgttacctc |
| | | acccgtaatctgtaa |

| | | |
|---|---|---|
| 25 | AAV8 | atggctgccgatggttatcttccagattggctcgaggacaacctctctgagggcattcgcgagtggtgggcgctgaa |
| | Swap17 | acctggagccccgaagcccaaagccaaccagcaaaagcaggacgacggccggggtctagtgcttcctggctaca |
| | (nt) | agtacctcggacccttcaacggactcgacaagggggagcccgtcaacgcggcggacgcagcggccctcgagcac |
| | | gacaaggcctacgaccagcagctgcaggcgggtgacaatccgtacctgcggtataaccacgccgacgccgagttt |
| | | caggagcgtctgcaagaagatacgtcttttgggggcaacctcgggcgagcagtcttccaggccaagaagcgggtt |
| | | ctcgaacctctcggtctggttgaggaaggcgctaagacggctcctggaaagaagagaccggtagagccatcaccc |
| | | cagcgttctccagactcctctacgggcatcggcaagaaaggccaacagcccgccagaaaaagactcaattttggtc |
| | | agactggcgactcagagtcagttccagaccctcaacctctcggagaacctccagcagcgccctctggtgtgggacc |
| | | taatacaatggctgcaggcggtggcgcaccaatggcagacaataacgaaggcgccgacggagtgggtagttcct |
| | | cgggaaattggcattgcgattccacatggctgggcgacagagtcatcaccaccagcacccgaacctgggccctgc |
| | | ccacctacaacaaccacctctacaagcaaatctccaacgggacatcgggaggagccaccaacgacaacacctact |
| | | tcggctacagcaccccctgggggtattttgactttaacagattccactgccacttttcaccacgtgactggcagcgac |
| | | tcatcaacaacaactggggattccggcccaagagactcagcttcaagctcttcaacatccaggtcaaggaggtcac |
| | | gcagaatgaaggcaccaagaccatcgccaataacctcaccagcaccatccaggtgtttacggactcggagtacca |
| | | gctgccgtacgttctcggctctgcccaccagggctgcctgcctccgttcccggcggacgtgttcatgattccccagta |
| | | cggctacctaacactcaacaacggtagtcaggccgtgggacgctcctccttctactgcctggaatactttccttcgca |
| | | gatgctgagaaccggcaacaacttccagtttacttacaccttcgaggacgtgcctttccacagcagctacgcccaca |
| | | gccagagcttggaccggctgatgaatcctctgattgaccagtacctgtactacttatccagaactcagtccacagga |
| | | ggaactcaaggtacccagcaattgttattttctcaagctgggcctgcaaacatgtcggctcaggccaagaactggct |
| | | gcctggaccttgctaccggcagcagcgagtctccacgacaacggggcaaaacaacaacagcaactttgcttggac |
| | | tgctggcaccaaatatcacctgaacggcagaaactcgttggctaatcccggcatcgccatggcaacacacaagga |
| | | cgacgaggaccgctttttcccatccagcggagtcctgatttttggaaaaactggagcaactaacaaaactacattgg |
| | | aaaatgtgttaatgacaaatgaagaagaaattcgtcctactaatcctgtagccacggaagaatacgggatagtca |
| | | gcagcaacttacaagcggctaatactgcagcccagacacaagttgtcaacaaccagggagccttacctggcatgg |
| | | tctggcagaaccgggacgtgtacctgcagggtcccatctgggccaagattcctcacacggacggcaacttccaccc |
| | | gtctccgctgatgggcggctttggcctgaaacatcctccgcctcagatcctgatcaagaacacgcctgtacctgcgg |
| | | atcctccgaccaccttcaaccagtcaaagctgaactctttcatcacgcaatacagcaccggacaggtcagcgtgga |
| | | aattgaatgggagctgcagaaggaaaacagcaagcgctggaaccccgagatccagtacacctccaactactaca |
| | | aatctacaagtgtggactttgctgttaatacagaaggcgtgtactctgaaccccgccccattggcacccgttacctca |
| | | cccgtaatctgtaa |
| 26 | AAV8 | atggctgccgatggttatcttccagattggctcgaggacaacctctctgagggcattcgcgagtggtgggcgctgaa |
| | Swap18 | acctggagccccgaagcccaaagccaaccagcaaaagcaggacgacggccggggtctagtgcttcctggctaca |
| | (nt) | agtacctcggacccttcaacggactcgacaagggggagcccgtcaacgcggcggacgcagcggccctcgagcac |
| | | gacaaggcctacgaccagcagctgcaggcgggtgacaatccgtacctgcggtataaccacgccgacgccgagttt |
| | | caggagcgtctgcaagaagatacgtcttttgggggcaacctcgggcgagcagtcttccaggccaagaagcgggtt |
| | | ctcgaacctctcggtctggttgaggaaggcgctaagacggctcctggaaagaagagaccggtagagccatcaccc |
| | | cagcgttctccagactcctctacgggcatcggcaagaaaggccaacagcccgccagaaaaagactcaattttggtc |
| | | agactggcgactcagagtcagttccagaccctcaacctctcggagaacctccagcagcgccctctggtgtgggacc |
| | | taatacaatggctgcaggcggtggcgcaccaatggcagacaataacgaaggcgccgacggagtgggtagttcct |
| | | cgggaaattggcattgcgattccacatggctgggcgacagagtcatcaccaccagcacccgaacctgggccctgc |
| | | ccacctacaacaaccacctctacaagcaaatctccaacgggacatcgggaggagccaccaacgacaacacctact |
| | | tcggctacagcaccccctgggggtattttgactttaacagattccactgccacttttcaccacgtgactggcagcgac |
| | | tcatcaacaacaactggggattccggcccaagagactcagcttcaagctcttcaacatccaggtcaaggaggtcac |
| | | gcagaatgaaggcaccaagaccatcgccaataacctcaccagcaccatccaggtgtttacggactcggagtacca |
| | | gctgccgtacgttctcggctctgcccaccagggctgcctgcctccgttcccggcggacgtgttcatgattccccagta |
| | | cggctacctaacactcaacaacggtagtcaggccgtgggacgctcctccttctactgcctggaatactttccttcgca |
| | | gatgctgagaaccggcaacaacttccagtttacttacaccttcgaggacgtgcctttccacagcagctacgcccaca |
| | | gccagagcttggaccggctgatgaatcctctgattgaccagtacctgtactacttatccagaactcagtccacagga |
| | | ggaactcaaggtacccagcaattgttattttctcaagctgggcctgcaaacatgtcggctcaggccaagaactggct |
| | | gcctggaccttgctaccggcagcagcgagtctccacgacactgtcgcaaaacaacaacagcaactttgcttggact |
| | | ggtgccaccaaatatcacctgaacggcagaaactcgttggttaatcccggcgtcgccatggcaacacacaaggac |
| | | gacgaggagcgctttttcccatccaacggaatcctgatttttggaaaaactggagcaactaacaaaactacattgg |
| | | aaaatgtgttaatgacaaatgaagaagaaattcgtcctactaatcctgtagccacggaagaatacgggatagtca |
| | | gcagcaacttacaagcggctaatactgcagcccagacacaagttgtcaacaaccagggagccttacctggcatgg |
| | | tctggcagaaccgggacgtgtacctgcagggtcccatctgggccaagattcctcacacggacggcaacttccaccc |
| | | gtctccgctgatgggcggctttggcctgaaacatcctccgcctcagatcctgatcaagaacacgcctgtacctgcgg |
| | | atcctccgaccaccttcaaccagtcaaagctgaactctttcatcacgcaatacagcaccggacaggtcagcgtgga |
| | | aattgaatgggagctgcagaaggaaaacagcaagcgctggaaccccgagatccagtacacctccaactactaca |
| | | aatctacaagtgtggactttgctgttaatacagaaggcgtgtactctgaaccccgccccattggcacccgttacctca |
| | | cccgtaatctgtaa |
| 27 | AAV8 | atggctgccgatggttatcttccagattggctcgaggacaacctctctgagggcattcgcgagtggtgggcgctgaa |
| | Swap19 | acctggagccccgaagcccaaagccaaccagcaaaagcaggacgacggccggggtctagtgcttcctggctaca |
| | (nt) | agtacctcggacccttcaacggactcgacaagggggagcccgtcaacgcggcggacgcagcggccctcgagcac |
| | | gacaaggcctacgaccagcagctgcagggggtgacaatccgtacctgcggtataaccacgccgacgccgagttt |
| | | caggagcgtctgcaagaagatacgtcttttgggggcaacctcgggcgagcagtcttccaggccaagaagcgggtt |
| | | ctcgaacctctcggtctggttgaggaaggcgctaagacggctcctggaaagaagagaccggtagagccatcaccc |
| | | cagcgttctccagactcctctacgggcatcggcaagaaaggccaacagcccgccagaaaaagactcaattttggtc |
| | | agactggcgactcagagtcagttccagaccctcaacctctcggagaacctccagcagcgccctctggtgtgggacc |
| | | taatacaatggctgcaggcggtggcgcaccaatggcagacaataacgaaggcgccgacggagtgggtagttcct |
| | | cgggaaattggcattgcgattccacatggctgggcgacagagtcatcaccaccagcacccgaacctgggccctgc |
| | | ccacctacaacaaccacctctacaagcaaatctccaacgggacatcgggaggagccaccaacgacaacacctact |
| | | tcggctacagcaccccctgggggtattttgactttaacagattccactgccacttttcaccacgtgactggcagcgac |
| | | tcatcaacaacaactggggattccggcccaagagactcagcttcaagctcttcaacatccaggtcaaggaggtcac |
| | | gcagaatgaaggcaccaagaccatcgccaataacctcaccagcaccatccaggtgtttacggactcggagtacca |
| | | gctgccgtacgttctcggctctgcccaccagggctgcctgcctccgttcccggcggacgtgttcatgattccccagta |
| | | cggctacctaacactcaacaacggtagtcaggccgtgggacgctcctccttctactgcctggaatactttccttcgca |
| | | gatgctgagaaccggcaacaacttccagtttacttacaccttcgaggacgtgcctttccacagcagctacgcccaca |
| | | gccagagcttggaccggctgatgaatcctctgattgaccagtacctgtactacttatccagaactcagtccacagga |
| | | ggaactcaaggtacccagcaattgttattttctcaagctgggcctgcaaacatgtcggctcaggccaagaactggct |
| | | gcctggaccttgctaccggcagcagcgagtctccacgacactgtcgcaaaacaacaacagcaactttgcttggact |
| | | ggtgccaccaaatatcacctgaacggcagaaactcgttggttaatcccggcgtcgccatggcaacacacaaggac |
| | | gacgaggaccgctttttcccatccagcggagtcctgatttttggaaaacagaatgcagcaagggacaacgctgact |
| | | actcagatgtgatgttgacaagtgaagaagaaattaagactactaatcctgtagccacggaagaatacgggatag |
| | | tcagcagcaacttacaagcggctaatactgcagcccagacacaagttgtcaacaaccagggagccttacctggca |
| | | tggtctggcagaaccgggacgtgtacctgcagggtcccatctgggccaagattcctcacacggacggcaacttcca |
| | | cccgtctccgctgatgggcggctttggcctgaaacatcctccgcctcagatcctgatcaagaacacgcctgtacctgc |
| | | ggatcctccgaccaccttcaaccagtcaaagctgaactctttcatcacgcaatacagcaccggacaggtcagcgtg |
| | | gaaattgaatgggagctgcagaaggaaaacagcaagcgctggaaccccgagatccagtacacctccaactacta |
| | | caaatctacaagtgtggactttgctgttaatacagaaggcgtgtactctgaaccccgccccattggcacccgttacct |
| | | cacccgtaatctgtaa |
| 28 | AAV8 | atggctgccgatggttatcttccagattggctcgaggacaacctctctgagggcattcgcgagtggtgggcgctgaa |
| | Swap20 | acctggagccccgaagcccaaagccaaccagcaaaagcaggacgacggccggggtctagtgcttcctggctaca |
| | (nt) | agtacctcggacccttcaacggactcgacaagggggagcccgtcaacgcggcggacgcagcggccctcgagcac |
| | | gacaaggcctacgaccagcagctgcagggggtgacaatccgtacctgcggtataaccacgccgacgccgagttt |
| | | caggagcgtctgcaagaagatacgtcttttgggggcaacctcgggcgagcagtcttccaggccaagaagcgggtt |
| | | ctcgaacctctcggtctggttgaggaaggcgctaagacggctcctggaaagaagagaccggtagagccatcaccc |
| | | cagcgttctccagactcctctacgggcatcggcaagaaaggccaacagcccgccagaaaaagactcaattttggtc |
| | | agactggcgactcagagtcagttccagaccctcaacctctcggagaacctccagcagcgccctctggtgtgggacc |
| | | taatacaatggctgcaggcggtggcgcaccaatggcagacaataacgaaggcgccgacggagtgggtagttcct |
| | | cgggaaattggcattgcgattccacatggctgggcgacagagtcatcaccaccagcacccgaacctgggccctgc |
| | | ccacctacaacaaccacctctacaagcaaatctccaacgggacatcgggaggagccaccaacgacaacacctact |
| | | tcggctacagcaccccctgggggtattttgactttaacagattccactgccacttttcaccacgtgactggcagcgac |
| | | tcatcaacaacaactggggattccggcccaagagactcagcttcaagctcttcaacatccaggtcaaggaggtcac |
| | | gcagaatgaaggcaccaagaccatcgccaataacctcaccagcaccatccaggtgtttacggactcggagtacca |
| | | gctgccgtacgttctcggctctgcccaccagggctgcctgcctccgttcccggcggacgtgttcatgattccccagta |
| | | cggctacctaacactcaacaacggtagtcaggccgtgggacgctcctccttctactgcctggaatactttccttcgca |
| | | gatgctgagaaccggcaacaacttccagtttacttacaccttcgaggacgtgcctttccacagcagctacgcccaca |
| | | gccagagcttggaccggctgatgaatcctctgattgaccagtacctgtactacttatccagaactcagtccacagga |
| | | ggaactcaaggtacccagcaattgttattttctcaagctgggcctgcaaacatgtcggctcaggccaagaactggct |
| | | gcctggaccttgctaccggcagcagcgagtctccacgacactgtcgcaaaacaacaacagcaactttgcttggact |
| | | ggtgccaccaaatatcacctgaacggcagaaactcgttggttaatcccggcgtcgccatggcaacacacaaggac |
| | | gacgaggaccgctttttcccatccagcggagtcctgatttttggaaaaactggagcaactaacaaaactacattgga |
| | | aaatgtgttaatgacaaatgaagaagaaattcgtcctactaatcctgtagccacggaagaatacgggatagtcgcc |
| | | gacaacttacaacagcagaatactgcaccccagataggaactgtcaacagccagggagccttacctggcatggtc |
| | | tggcagaaccgggacgtgtacctgcagggtcccatctgggccaagattcctcacacggacggcaacttccacccgt |
| | | ctccgctgatgggcggctttggcctgaaacatcctccgcctcagatcctgatcaagaacacgcctgtacctgcggat |
| | | cctccgaccaccttcaaccagtcaaagctgaactctttcatcacgcaatacagcaccggacaggtcagcgtggaaa |
| | | ttgaatgggagctgcagaaggaaaacagcaagcgctggaaccccgagatccagtacacctccaactactacaaa |
| | | tctacaagtgtggactttgctgttaatacagaaggcgtgtactctgaaccccgccccattggcacccgttacctcacc |
| | | cgtaatctgtaa |
| 29 | AAV8 | atggctgccgatggttatcttccagattggctcgaggacaacctctctgagggcattcgcgagtggtgggcgctgaa |
| | Swap21 | acctggagccccgaagcccaaagccaaccagcaaaagcaggacgacggccggggtctagtgcttcctggctaca |
| | (nt) | agtacctcggacccttcaacggactcgacaagggggagcccgtcaacgcggcggacgcagcggccctcgagcac |
| | | gacaaggcctacgaccagcagctgcaggcgggtgacaatccgtacctgcggtataaccacgccgacgccgagttt |
| | | caggagcgtctgcaagaagatacgtcttttgggggcaacctcgggcgagcagtcttccaggccaagaagcgggtt |
| | | ctcgaacctctcggtctggttgaggaaggcgctaagacggctcctggaaagaagagaccggtagagccatcaccc |
| | | cagcgttctccagactcctctacgggcatcggcaagaaaggccaacagcccgccagaaaaagactcaattttggtc |
| | | agactggcgactcagagtcagttccagaccctcaacctctcggagaacctccagcagcgccctctggtgtgggacc |
| | | taatacaatggctgcaggcggtggcgcaccaatggcagacaataacgaaggcgccgacggagtgggtagttcct |
| | | cgggaaattggcattgcgattccacatggctgggcgacagagtcatcaccaccagcacccgaacctgggccctgc |
| | | ccacctacaacaaccacctctacaagcaaatctccaacgggacatcgggaggagccaccaacgacaacacctact |
| | | tcggctacagcaccccctgggggtattttgactttaacagattccactgccacttttcaccacgtgactggcagcgac |
| | | tcatcaacaacaactggggattccggcccaagagactcagcttcaagctcttcaacatccaggtcaaggaggtcac |
| | | gcagaatgaaggcaccaagaccatcgccaataacctcaccagcaccatccaggtgtttacggactcggagtacca |
| | | gctgccgtacgttctcggctctgcccaccagggctgcctgcctccgttcccggcggacgtgttcatgattccccagta |
| | | cggctacctaacactcaacaacggtagtcaggccgtgggacgctcctccttctactgcctggaatactttccttcgca |
| | | gatgctgagaaccggcaacaacttccagtttacttacaccttcgaggacgtgcctttccacagcagctacgcccaca |
| | | gccagagcttggaccggctgatgaatcctctgattgaccagtacctgtactacttatccagaactcagtccacagga |
| | | ggaactcaaggtacccagcaattgttattttctcaagctgggcctgcaaacatgtcggctcaggccaagaactggct |
| | | gcctggaccttgctaccggcagcagcgagtctccacgacactgtcgcaaaacaacaacagcaactttgcttggact |
| | | ggtgccaccaaatatcacctgaacggcagaaactcgttggttaatcccggcgtcgccatggcaacacacaaggac |
| | | gacgaggaccgtttttttcccagtagcggggtcctgatttttggcaaacaaaatgctgccagagacaatgcggatta |
| | | cagcgatgtcatgctcaccagcgaggaagaaatcaaaaccactaaccctgtggctacagaggaatacggtatcgt |
| | | ggcagataacttgcagcagcaaaacacggctcctcaaattggaactgtcaacagccagggggccttacccggtat |
| | | ggtctggcagaaccgggacgtgtacctgcagggtcccatctgggccaagattcctcacacggacggcaacttccac |
| | | ccgtctccgctgatgggcggctttggcctgaaacatcctccgcctcagatcctgatcaagaacacgcctgtacctgc |
| | | ggatcctccgaccaccttcaaccagtcaaagctgaactctttcatcacgcaatacagcaccggacaggtcagcgtg |
| | | gaaattgaatgggagctgcagaaggaaaacagcaagcgctggaaccccgagatccagtacacctccaactacta |
| | | caaatctacaagtgtggactttgctgttaatacagaaggcgtgtactctgaaccccgccccattggcacccgttacct |
| | | cacccgtaatctgtaa |
| 30 | AAV8 | atggctgccgatggttatcttccagattggctcgaggacaacctctctgagggcattcgcgagtggtgggcgctgaa |
| | Swap22 | acctggagccccgaagcccaaagccaaccagcaaaagcaggacgacggccggggtctagtgcttcctggctaca |
| | (nt) | agtacctcggacccttcaacggactcgacaagggggagcccgtcaacgcggcggacgcagcggccctcgagcac |
| | | gacaaggcctacgaccagcagctgcaggcgggtgacaatccgtacctgcggtataaccacgccgacgccgagttt |
| | | caggagcgtctgcaagaagatacgtcttttgggggcaacctcgggcgagcagtcttccaggccaagaagcgggtt |
| | | ctcgaacctctcggtctggttgaggaaggcgctaagacggctcctggaaagaagagaccggtagagccatcaccc |
| | | cagcgttctccagactcctctacgggcatcggcaagaaaggccaacagcccgccagaaaaagactcaattttggtc |
| | | agactggcgactcagagtcagttccagaccctcaacctctcggagaacctccagcagcgccctctggtgtgggacc |
| | | taatacaatggctgcaggcggtggcgcaccaatggcagacaataacgaaggcgccgacggagtgggtagttcct |
| | | cgggaaattggcattgcgattccacatggctgggcgacagagtcatcaccaccagcacccgaacctgggccctgc |
| | | ccacctacaacaaccacctctacaagcaaatctccaacgggacatcgggaggagccaccaacgacaacacctact |
| | | tcggctacagcaccccctgggggtattttgactttaacagattccactgccacttttcaccacgtgactggcagcgac |
| | | tcatcaacaacaactggggattccggcccaagagactcagcttcaagctcttcaacatccaggtcaaggaggtcac |
| | | gcagaatgaaggcaccaagaccatcgccaataacctcaccagcaccatccaggtgtttacggactcggagtacca |
| | | gctgccgtacgttctcggctctgcccaccagggctgcctgcctccgttcccggcggacgtgttcatgattccccagta |
| | | cggctacctaacactcaacaacggtagtcaggccgtgggacgctcctccttctactgcctggaatactttccttcgca |
| | | gatgctgagaaccggcaacaacttccagtttacttacaccttcgaggacgtgcctttccacagcagctacgcccaca |
| | | gccagagcttggaccggctgatgaatcctctgattgaccagtacctgtactacttatccagaactcagtccacagga |
| | | ggaactcaaggtacccagcaattgttattttctcaagctgggcctgcaaacatgtcggctcaggccaagaactggct |
| | | gcctggaccttgctaccggcagcagcgagtctccacgacactgtcgcaaaacaacaacagcaactttgcttggact |
| | | ggtgccaccaaatatcacctgaacggcagaaactcgttggttaatcccggcgtcgccatggcaacacacaaggac |
| | | gacgaggagcgtttttttcccagtaacgggatcctgatttttggcaaaactggtgccacaaacaaaacgactttgga |
| | | gaatgtcttgatgaccaacgaggaagaaatcagacccactaaccctgtggctacagaggaatacggtatcgtggc |
| | | agataacttgcagcagcaaaacacggctcctcaaattggaactgtcaacagccagggggccttacccggtatggt |
| | | ctggcagaaccgggacgtgtacctgcagggtcccatctgggccaagattcctcacacggacggcaacttccaccc |
| | | gtctccgctgatgggcggctttggcctgaaacatcctccgcctcagatcctgatcaagaacacgcctgtacctgcgg |
| | | atcctccgaccaccttcaaccagtcaaagctgaactctttcatcacgcaatacagcaccggacaggtcagcgtgga |
| | | aattgaatgggagctgcagaaggaaaacagcaagcgctggaaccccgagatccagtacacctccaactactaca |
| | | aatctacaagtgtggactttgctgttaatacagaaggcgtgtactctgaaccccgccccattggcacccgttacctca |
| | | cccgtaatctgtaa |
| 31 | AAV8 | atggctgccgatggttatcttccagattggctcgaggacaacctctctgagggcattcgcgagtggtgggcgctgaa |
| | Swap23 | acctggagccccgaagcccaaagccaaccagcaaaagcaggacgacggccggggtctagtgcttcctggctaca |
| | (nt) | agtacctcggacccttcaacggactcgacaagggggagcccgtcaacgcggcggacgcagcggccctcgagcac |
| | | gacaaggcctacgaccagcagctgcaggcgggtgacaatccgtacctgcggtataaccacgccgacgccgagttt |
| | | caggagcgtctgcaagaagatacgtcttttgggggcaacctcgggcgagcagtcttccaggccaagaagcgggtt |
| | | ctcgaacctctcggtctggttgaggaaggcgctaagacggctcctggaaagaagagaccggtagagccatcaccc |
| | | cagcgttctccagactcctctacgggcatcggcaagaaaggccaacagcccgccagaaaaagactcaattttggtc |
| | | agactggcgactcagagtcagttccagaccctcaacctctcggagaacctccagcagcgccctctggtgtgggacc |
| | | taatacaatggctgcaggcggtggcgcaccaatggcagacaataacgaaggcgccgacggagtgggtagttcct |
| | | cgggaaattggcattgcgattccacatggctgggcgacagagtcatcaccaccagcacccgaacctgggccctgc |
| | | ccacctacaacaaccacctctacaagcaaatctccaacgggacatcgggaggagccaccaacgacaacacctact |
| | | tcggctacagcaccccctgggggtattttgactttaacagattccactgccacttttcaccacgtgactggcagcgac |
| | | tcatcaacaacaactggggattccggcccaagagactcagcttcaagctcttcaacatccaggtcaaggaggtcac |
| | | gcagaatgaaggcaccaagaccatcgccaataacctcaccagcaccatccaggtgtttacggactcggagtacca |
| | | gctgccgtacgttctcggctctgcccaccagggctgcctgcctccgttcccggcggacgtgttcatgattccccagta |
| | | cggctacctaacactcaacaacggtagtcaggccgtgggacgctcctccttctactgcctggaatactttccttcgca |
| | | gatgctgagaaccggcaacaacttccagtttacttacaccttcgaggacgtgcctttccacagcagctacgcccaca |
| | | gccagagcttggaccggctgatgaatcctctgattgaccagtacctgtactacttatccagaactcagtccacagga |
| | | ggaactcaaggtacccagcaattgttattttctcaagctgggcctgcaaacatgtcggctcaggccaagaactggct |
| | | gcctggaccttgctaccggcagcagcgagtctccacgacactgtcgcaaaacaacaacagcaactttgcttggact |
| | | ggtgccaccaaatatcacctgaacggcagaaactcgttggttaatcccggcgtcgccatggcaacacacaaggac |
| | | gacgaggagcgtttttttcccagtaacgggatcctgatttttggcaaacaaaatgctgccagagacaatgcggatta |
| | | cagcgatgtcatgctcaccagcgaggaagaaatcaaaaccactaaccctgtggctacagaggaatacggtatcgt |
| | | gtcatctaacttgcaggcggcaaacacggctgctcaaactcaagttgtcaacaaccagggggccttacccggtatg |
| | | gtctggcagaaccgggacgtgtacctgcagggtcccatctgggccaagattcctcacacggacggcaacttccacc |
| | | cgtctccgctgatgggcggctttggcctgaaacatcctccgcctcagatcctgatcaagaacacgcctgtacctgcg |
| | | gatcctccgaccaccttcaaccagtcaaagctgaactctttcatcacgcaatacagcaccggacaggtcagcgtgg |
| | | aaattgaatgggagctgcagaaggaaaacagcaagcgctggaaccccgagatccagtacacctccaactactac |
| | | aaatctacaagtgtggactttgctgttaatacagaaggcgtgtactctgaaccccgccccattggcacccgttacctc |
| | | acccgtaatctgtaa |
| 47 | AAV | MAADGYLPDWLEDTLSEGIREWWALKPGAPQPKANQQHQDNGRGLVLPGYKYLGPFNGL |
| | C11.12.253 | DKGEPVNEADAAALEHDKAYDKQLEQGDNPYLKYNHADAEFQERLQEDTSFGGNLGRAVF |
| | R4.1 | QAKKRILEPLGLVEEAAKTAPGKKRPVEPSPQRSPDSSTGIGKKGQQPARKRLNFGQTGDS |
| | | ESVPDPQPLGEPPAAPSSVGSGTVAAGGGAPMADNNEGADGVGNASGNWHCDSTWLGD |
| | | RVITTSTRTWALPTYNNHLYKQISSQSGASNDNHYFGYSTPWGYFDFNRFHCHFSPRDWQ |
| | | RLINNNWGFRPKRLSFKLFNIQVKEVTTNDGVTTIANNLTSTVQVFSDSEYQLPYVLGSAHQ |
| | | GCLPPFPADVFMIPQYGYLTLNNGSQAVGRSSFYCLEYFPSQMLRTGNNFEFSYTFEDVPFH |
| | | SSYAHSQSLDRLMNPLIDQYLYYLSRTQSTGGTQGTQQLLFSQAGPANMSAQAKNWLPGP |
| | | CYRQQRVSTTLSQNNNSNFAWTGATKYHLNGRNSLVNPGVAMATHKDDEDRFFPSSGVLI |
| | | FGKQGSANDATHEDVMLTDEEEIRPTNPVATEEYGIVSSNLQAANTAAQTQVVNNQGALP |
| | | GMVWQNRDVYLQGPIWAKIPHTDGNFHPSPLMGGFGLKHPPPQILIKNTPVPANPPEVFTP |
| | | AKFASFITQYSTGQVSVEIEWELQKENSKRWNPEIQYTSNYNKSVSVDFTVDTNGVYSEPR |
| | | PIGTRYLTRNL |
| 48 | AAV | MAADGYLPDWLEDTLSEGIREWWALKPGAPQPKANQQHQDNGRGLVLPGYKYLGPFNGL |
| | C11.12.253 | DKGEPVNEADAAALEHDKAYDKQLEQGDNPYLKYNHADAEFQERLQEDTSFGGNLGRAVF |
| | R4.3 | QAKKRILEPLGLVEEAAKTAPGKKRPVEPSPQRSPDSSTGIGKKGQQPARKRLNFGQTGDS |
| | | ESVPDPQPLGEPPAAPSSVGSGTVAAGGGAPMADNNEGADGVGNASGNWHCDSTWLGD |
| | | RVITTSTRTWALPTYNNHLYKQISSQSGASNDNHYFGYSTPWGYFDFNRFHCHFSPRDWQ |
| | | RLINNNWGFRPKRLSFKLFNIQVKEVTTNDGVTTIANNLTSTVQVFSDSEYQLPYVLGSAHQ |
| | | GCLPPFPADVFMIPQYGYLTLNNGSQAVGRSSFYCLEYFPSQMLRTGNNFEFSYTFEDVPFH |
| | | SSYAHSQSLDRLMNPLIDQYLYYLSRTQSTGGTQGTQQLLFSQAGPANMSAQAKNWLPGP |
| | | CYRQQRVSTTLSQNNNSNFAWTGATKYHLNGRNSLVNPGVAMATHKDDEDRFFPSSGVLI |
| | | FGKTGATNKTTLENVLMTNEEEIRPTNPVATEEYGIVSSNLQNSNTAPEIANVNNQGALPGM |
| | | VWQNRDVYLQGPIWAKIPHTDGNFHPSPLMGGFGLKHPPPQILIKNTPVPANPPEVFTPAKF |
| | | ASFITQYSTGQVSVEIEWELQKENSKRWNPEIQYTSNYNKSVSVDFTVDTNGVYSEPRPIG |
| | | TRYLTRNL |
| 49 | AAV | MAADGYLPDWLEDTLSEGIREWWALKPGAPQPKANQQHQDNGRGLVLPGYKYLGPFNGL |
| | C11.12.253 | DKGEPVNEADAAALEHDKAYDKQLEQGDNPYLKYNHADAEFQERLQEDTSFGGNLGRAVF |
| | R4.6 | QAKKRILEPLGLVEEAAKTAPGKKRPVEPSPQRSPDSSTGIGKKGQQPARKRLNFGQTGDS |
| | | ESVPDPQPLGEPPAAPSSVGSGTVAAGGGAPMADNNEGADGVGNASGNWHCDSTWLGD |
| | | RVITTSTRTWALPTYNNHLYKQISSQSGASNDNHYFGYSTPWGYFDFNRFHCHFSPRDWQ |
| | | RLINNNWGFRPKRLSFKLFNIQVKEVTTNDGVTTIANNLTSTVQVFSDSEYQLPYVLGSAHQ |
| | | GCLPPFPADVFMIPQYGYLTLNNGSQAVGRSSFYCLEYFPSQMLRTGNNFEFSYTFEDVPFH |
| | | SSYAHSQSLDRLMNPLIDQYLYYLSRTQSTGGTQGTQQLLFSQAGPANMSAQAKNWLPGP |
| | | CYRQQRVSTTLSQNNNSNFAWTGATKYHLNGRNSLVNPGVAMATHKDDEDRFFPSSGVLI |
| | | FGKQGSANDATHEDVMLTDEEEIRPTNPVATEEYGIVSSNLQNSNTAPEIANVNNQGALPG |
| | | MVWQNRDVYLQGPIWAKIPHTDGNFHPSPLMGGFGLKHPPPQILIKNTPVPANPPEVFTPA |
| | | KFASFITQYSTGQVSVEIEWELQKENSKRWNPEIQYTSNYNKSVSVDFTVDTNGVYSEPRP |
| | | IGTRYLTRNL |
| 50 | AAV | MAADGYLPDWLEDTLSEGIREWWALKPGAPQPKANQQHQDNGRGLVLPGYKYLGPFNGL |
| | C11.12.253 | DKGEPVNEADAAALEHDKAYDKQLEQGDNPYLKYNHADAEFQERLQEDTSFGGNLGRAVF |
| | R4.9 | QAKKRILEPLGLVEEAAKTAPGKKRPVEPSPQRSPDSSTGIGKKGQQPARKRLNFGQTGDS |
| | | ESVPDPQPLGEPPAAPSSVGSGTVAAGGGAPMADNNEGADGVGNASGNWHCDSTWLGD |
| | | RVITTSTRTWALPTYNNHLYKQISSQSGASNDNHYFGYSTPWGYFDFNRFHCHFSPRDWQ |
| | | RLINNNWGFRPKRLSFKLFNIQVKEVTTNDGVTTIANNLTSTVQVFSDSEYQLPYVLGSAHQ |
| | | GCLPPFPADVFMIPQYGYLTLNNGSQAVGRSSFYCLEYFPSQMLRTGNNFEFSYTFEDVPFH |
| | | SSYAHSQSLDRLMNPLIDQYLYYLSRTQSTGGTQGTQQLLFSQAGPANMSAQAKNWLPGP |
| | | CYRQQRVSTTLSQNNNSNFAWTGATKYHLNGRNSLVNPGVAMATHKDDEDRFFPSSGVLI |
| | | FGKKNTNTNNVNYENVMMTNEEEIRPTNPVATEEYGIVSSNLQAANTAAQTQVVNNQGALP |
| | | GMVWQNRDVYLQGPIWAKIPHTDGNFHPSPLMGGFGLKHPPPQILIKNTPVPANPPEVFTP |
| | | AKFASFITQYSTGQVSVEIEWELQKENSKRWNPEIQYTSNYNKSVSVDFTVDTNGVYSEPR |
| | | PIGTRYLTRNL |
| 51 | AAV | MAADGYLPDWLEDTLSEGIREWWALKPGAPQPKANQQHQDNGRGLVLPGYKYLGPFNGL |
| | C11.12.269 | DKGEPVNEADAAALEHDKAYDKQLEQGDNPYLKYNHADAEFQERLQEDTSFGGNLGRAVF |
| | R4.1 | QAKKRILEPLGLVEEAAKTAPGKKRPVEPSPQRSPDSSTGIGKKGQQPARKRLNFGQTGDS |
| | | ESVPDPQPLGEPPAAPSSVGSGTVAAGGGAPMADNNEGADGVGNASGNWHCDSTWLGD |
| | | RVITTSTRTWALPTYNNHLYKQISSQSGASNDNHYFGYSTPWGYFDFNRFHCHFSPRDWQ |
| | | RLINNNWGFRPKRLSFKLFNIQVKEVTTNDGVTTIANNLTSTVQVFSDSEYQLPYVLGSAHQ |
| | | GCLPPFPADVFMIPQYGYLTLNNGSQAVGRSSFYCLEYFPSQMLRTGNNFEFSYTFEDVPFH |
| | | SSYAHSQSLDRLMNPLIDQYLYYLGRTQATPGGTTGQNTLLFSQAGPANMSAQAKNWLPG |
| | | PCYRQQRVSTTLSQNNNSNFAWTGATKYHLNGRNSLVNPGVAMATHKDDEDRFFPSSGV |
| | | LIFGKTGATNKTTLENVLMTNEEEIRPTNPVATEEYGIVSSNLQAANTAAQTQVVNNQGALP |
| | | GMVWQNRDVYLQGPIWAKIPHTDGNFHPSPLMGGFGLKHPPPQILIKNTPVPANPPEVFTP |

| | | |
|---|---|---|
| | | AKFASFITQYSTGQVSVEIEWELQKENSKRWNPEIQYTSNYNKSVSVDFTVDTNGVYSEPR |
| | | PIGTRYLTRNL |
| 52 | AAV | MAADGYLPDWLEDTLSEGIREWWALKPGAPQPKANQQHQDNGRGLVLPGYKYLGPFNGL |
| | C11.12.269 | DKGEPVNEADAAALEHDKAYDKQLEQGDNPYLKYNHADAEFQERLQEDTSFGGNLGRAVF |
| | R4.3 | QAKKRILEPLGLVEEAAKTAPGKKRPVEPSPQRSPDSSTGIGKKGQQPARKRLNFGQTGDS |
| | | ESVPDPQPLGEPPAAPSSVGSGTVAAGGGAPMADNNEGADGVGNASGNWHCDSTWLGD |
| | | RVITTSTRTWALPTYNNHLYKQISSQSGASNDNHYFGYSTPWGYFDFNRFHCHFSPRDWQ |
| | | RLINNNWGFRPKRLSFKLFNIQVKEVTTNDGVTTIANNLTSTVQVFSDSEYQLPYVLGSAHQ |
| | | GCLPPFPADVFMIPQYGYLTLNNGSQAVGRSSFYCLEYFPSQMLRTGNNFEFSYTFEDVPFH |
| | | SSYAHSQSLDRLMNPLIDQYLYYLSRTQSTGGTQGTQQLLFSQAGPANMSAQAKNWLPGP |
| | | CYRQQRVSTTLSQNNNSNFAWTGATKYHLNGRNSLVNPGVAMATHKDDEDRFFPSSGVLI |
| | | FGKTGATNKTTLENVLMTNEEEIRPTNPVATEEYGIVSSNLQKENTPAQIEAVNNQGALPGM |
| | | VWQNRDVYLQGPIWAKIPHTDGNFHPSPLMGGFGLKHPPPQILIKNTPVPANPPEVFTPAKF |
| | | ASFITQYSTGQVSVEIEWELQKENSKRWNPEIQYTSNYNKSVSVDFTVDTNGVYSEPRPIG |
| | | TRYLTRNL |
| 53 | AAV | MAADGYLPDWLEDTLSEGIREWWALKPGAPQPKANQQHQDNGRGLVLPGYKYLGPFNGL |
| | C11.12.269 | DKGEPVNEADAAALEHDKAYDKQLEQGDNPYLKYNHADAEFQERLQEDTSFGGNLGRAVF |
| | R4.5 | QAKKRILEPLGLVEEAAKTAPGKKRPVEPSPQRSPDSSTGIGKKGQQPARKRLNFGQTGDS |
| | | ESVPDPQPLGEPPAAPSSVGSGTVAAGGGAPMADNNEGADGVGNASGNWHCDSTWLGD |
| | | RVITTSTRTWALPTYNNHLYKQISSQSGASNDNHYFGYSTPWGYFDFNRFHCHFSPRDWQ |
| | | RLINNNWGFRPKRLSFKLFNIQVKEVTTNDGVTTIANNLTSTVQVFSDSEYQLPYVLGSAHQ |
| | | GCLPPFPADVFMIPQYGYLTLNNGSQAVGRSSFYCLEYFPSQMLRTGNNFEFSYTFEDVPFH |
| | | SSYAHSQSLDRLMNPLIDQYLYYLSRTQSTGGTQGTQQLLFSQAGPANMSAQAKNWLPGP |
| | | CYRQQRVSTTLSQNNNSNFAWTGATKYHLNGRNSLVNPGVAMATHKDDEDRFFPSSGVLI |
| | | FGKQSSSGAKVNLEGVLLTGEEEIRPTNPVATEEYGIVSSNLQAANTAAQTQVVNNQGALP |
| | | GMVWQNRDVYLQGPIWAKIPHTDGNFHPSPLMGGFGLKHPPPQILIKNTPVPANPPEVFTP |
| | | AKFASFITQYSTGQVSVEIEWELQKENSKRWNPEIQYTSNYNKSVSVDFTVDTNGVYSEPR |
| | | PIGTRYLTRNL |
| 54 | AAV | MAADGYLPDWLEDTLSEGIREWWALKPGAPQPKANQQHQDNGRGLVLPGYKYLGPFNGL |
| | C11.12.269 | DKGEPVNEADAAALEHDKAYDKQLEQGDNPYLKYNHADAEFQERLQEDTSFGGNLGRAVF |
| | R4.6 | QAKKRILEPLGLVEEAAKTAPGKKRPVEPSPQRSPDSSTGIGKKGQQPARKRLNFGQTGDS |
| | | ESVPDPQPLGEPPAAPSSVGSGTVAAGGGAPMADNNEGADGVGNASGNWHCDSTWLGD |
| | | RVITTSTRTWALPTYNNHLYKQISSQSGASNDNHYFGYSTPWGYFDFNRFHCHFSPRDWQ |
| | | RLINNNWGFRPKRLSFKLFNIQVKEVTTNDGVTTIANNLTSTVQVFSDSEYQLPYVLGSAHQ |
| | | GCLPPFPADVFMIPQYGYLTLNNGSQAVGRSSFYCLEYFPSQMLRTGNNFEFSYTFEDVPFH |
| | | SSYAHSQSLDRLMNPLIDQYLYYLSKTQATSGTKDTNQLLFSQAGPANMSAQAKNWLPGP |
| | | CYRQQRVSTTLSQNNNSNFAWTGATKYHLNGRNSLVNPGVAMATHKDDEDRFFPSSGVLI |
| | | FGKTGATNKTTLENVLMTNEEEIRPTNPVATEEYGIVSSNLQAANTAAQTQVVNNQGALPG |
| | | MVWQNRDVYLQGPIWAKIPHTDGNFHPSPLMGGFGLKHPPPQILIKNTPVPANPPEVFTPA |
| | | KFASFITQYSTGQVSVEIEWELQKENSKRWNPEIQYTSNYNKSVSVDFTVDTNGVYSEPRP |
| | | IGTRYLTRNL |
| 55 | AAV | MAADGYLPDWLEDTLSEGIREWWALKPGAPQPKANQQHQDNGRGLVLPGYKYLGPFNGL |
| | C11.12.269 | DKGEPVNEADAAALEHDKAYDKQLEQGDNPYLKYNHADAEFQERLQEDTSFGGNLGRAVF |
| | R4.20 | QAKKRILEPLGLVEEAAKTAPGKKRPVEPSPQRSPDSSTGIGKKGQQPARKRLNFGQTGDS |
| | | ESVPDPQPLGEPPAAPSSVGSGTVAAGGGAPMADNNEGADGVGNASGNWHCDSTWLGD |
| | | RVITTSTRTWALPTYNNHLYKQISSQSGASNDNHYFGYSTPWGYFDFNRFHCHFSPRDWQ |
| | | RLINNNWGFRPKRLSFKLFNIQVKEVTTNDGVTTIANNLTSTVQVFSDSEYQLPYVLGSAHQ |
| | | GCLPPFPADVFMIPQYGYLTLNNGSQAVGRSSFYCLEYFPSQMLRTGNNFEFSYTFEDVPFH |
| | | SSYAHSQSLDRLMNPLIDQYLYYLSRTQSTGGTQGTQQLLFSQAGPANMSAQAKNWLPGP |
| | | CYRQQRVSTTLSQNNNSNFAWTGATKYHLNGRNSLVNPGVAMATHKDDEDRFFPSSGVLI |
| | | FGKTGATNKTTLENVLMTNEEEIRPTNPVATEEYGIVSSNLQTGNTAPETLIVNHQGALPGM |
| | | VWQNRDVYLQGPIWAKIPHTDGNFHPSPLMGGFGLKHPPPQILIKNTPVPANPPEVFTPAKF |
| | | ASFITQYSTGQVSVEIEWELQKENSKRWNPEIQYTSNYNKSVSVDFTVDTNGVYSEPRPIG |
| | | TRYLTRNL |
| 56 | AAV | ATGGCTGCCGATGGTTATCTTCCAGATTGGCTCGAGGACACTCTCTCTGAAGGCATTCG |
| | C11.12.253 | CGAGTGGTGGGCGCTGAAACCTGGAGCTCCACAACCCAAGGCCAACCAACAGCATCAG |
| | R4.1 | GACAACGGCAGGGGTCTTGTGCTTCCTGGGTACAAGTACCTCGGACCCTTCAACGGACT |
| | | CGACAAGGGAGAGCCGGTCAACGAGGCAGACGCCGCGGCCCTCGAGCACGACAAGGC |
| | | CTACGACAAGCAGCTCGAGCAGGGGGACAACCCGTACCTCAAGTACAACCACGCCGAC |
| | | GCCGAGTTTCAGGAGCGTCTTCAAGAAGATACGTCTTTTGGGGGCAACCTTGGCAGAGC |
| | | AGTCTTCCAGGCCAAAAAGAGGATCCTTGAGCCTCTTGGTCTGGTTGAGGAAGCTGCTA |
| | | AGACGGCTCCTGGAAAGAAGAGACCGGTAGAACCGTCACCTCAGCGTTCCCCCGACTCC |
| | | TCCACGGGCATCGGCAAGAAAGGCCAGCAGCCCGCCAGAAAGAGACTCAATTTCGGTC |
| | | AGACTGGCGACTCAGAGTCAGTCCCCGACCCTCAACCTCTCGGAGAACCTCCAGCAGCG |
| | | CCCTCTAGTGTGGGATCTGGTACAGTGGCTGCAGGCGGTGGCGCACCAATGGCAGACA |
| | | ATAACGAAGGTGCCGACGGAGTGGGTAATGCCTCAGGAAATTGGCATTGCGATTCCACA |
| | | TGGCTGGGCGACAGAGTCATCACCACCAGCACCAGAACCTGGGCCCTGCCCACTTACAA |
| | | CAACCATCTCTACAAGCAAATCTCCAGCCAATCAGGAGCTTCAAACGACAACCACTACTT |
| | | CGGCTACAGCACCCCCTGGGGGTATTTTGACTTTAACAGATTCCACTGCCACTTTTCACC |
| | | ACGTGACTGGCAGCGACTCATCAACAACAACTGGGGATTCCGGCCCAAGAGACTCAGCT |
| | | TCAAGCTCTTCAACATCCAAGTCAAGGAGGTCACGACGAATGATGGCGTCACGACCATC |
| | | GCTAATAACCTTACCAGCACGGTTCAAGTCTTCTCGGACTCGGAGTACCAGCTTCCGTAC |
| | | GTCCTCGGCTCTGCGCACCAGGGCTGCCTCCCTCCGTTCCCGGCGGACGTGTTCATGAT |
| | | TCCGCAGTACGGCTACCTAACGCTCAACAATGGCAGCCAGGCAGTGGGACGGTCATCCT |
| | | TTTACTGCCTGGAATATTTTCCATCTCAAATGCTGCGAACTGGAAACAATTTTGAATTCAG |
| | | CTACACCTTCGAGGACGTGCCTTTCCACAGCAGCTACGCACACAGCCAGAGCTTGGACC |
| | | GACTGATGAATCCTCTCATTGACCAGTACCTGTACTACTTATCCAGAACTCAGTCCACAG |
| | | GAGGAACTCAAGGTACCCAGCAATTGTTATTTTCTCAAGCTGGGCCTGCAAACATGTCG |
| | | GCTCAGGCCAAGAACTGGCTGCCTGGACCTTGCTACCGGCAGCAGCGAGTCTCCACGA |
| | | CACTGTCGCAAAACAACAACAGCAACTTTGCTTGGACTGGTGCCACCAAATATCACCTGA |
| | | ACGGCAGAAACTCGTTGGTTAATCCCGGCGTCGCCATGGCAACTCACAAGGACGACGA |
| | | GGACCGCTTTTTCCCATCCAGCGGGGTACTAATCTTCGGTAAACAGGGCTCGGCCAACG |
| | | ACGCGACCCACGAGGACGTGATGCTCACAGACGAAGAAGAAATTCGTCCTACTAATCCT |
| | | GTAGCCACGGAAGAATACGGGATAGTCAGCAGCAACTTACAAGCGGCTAATACTGCAGC |
| | | CCAGACACAAGTTGTCAACAACCAGGGAGCCTTACCTGGCATGGTCTGGCAGAACCGG |
| | | GACGTGTACCTGCAGGGTCCCATCTGGGCCAAGATTCCTCACACGGATGGCAACTTTCA |
| | | CCCGTCTCCTTTGATGGGCGGCTTTGGACTTAAACATCCGCCTCCTCAGATCCTGATCAA |
| | | GAACACTCCCGTTCCCGCTAATCCTCCGGAGGTGTTTACTCCTGCCAAGTTTGCTTCGTT |
| | | CATCACACAGTACAGCACCGGACAAGTCAGCGTGGAAATCGAGTGGGAGCTGCAGAAG |
| | | GAAAACAGCAAGCGCTGGAACCCGGAGATTCAGTACACTTCAAACTACAACAAGTCTGT |
| | | TAGTGTGGACTTTACTGTAGACACTAATGGCGTGTATTCAGAGCCTCGCCCCATTGGCAC |
| | | CAGATACCTGACTCGTAATCTGTAA |
| 57 | AAV | ATGGCTGCCGATGGTTATCTTCCAGATTGGCTCGAGGACACTCTCTCTGAAGGCATTCG |
| | C11.12.253 | CGAGTGGTGGGCGCTGAAACCTGGAGCTCCACAACCCAAGGCCAACCAACAGCATCAG |
| | R4.3 | GACAACGGCAGGGGTCTTGTGCTTCCTGGGTACAAGTACCTCGGACCCTTCAACGGACT |
| | | CGACAAGGGAGAGCCGGTCAACGAGGCAGACGCCGCGGCCCTCGAGCACGACAAGGC |
| | | CTACGACAAGCAGCTCGAGCAGGGGGACAACCCGTACCTCAAGTACAACCACGCCGAC |
| | | GCCGAGTTTCAGGAGCGTCTTCAAGAAGATACGTCTTTTGGGGGCAACCTTGGCAGAGC |
| | | AGTCTTCCAGGCCAAAAAGAGGATCCTTGAGCCTCTTGGTCTGGTTGAGGAAGCTGCTA |
| | | AGACGGCTCCTGGAAAGAAGAGACCGGTAGAACCGTCACCTCAGCGTTCCCCCGACTCC |
| | | TCCACGGGCATCGGCAAGAAAGGCCAGCAGCCCGCCAGAAAGAGACTCAATTTCGGTC |
| | | AGACTGGCGACTCAGAGTCAGTCCCCGACCCTCAACCTCTCGGAGAACCTCCAGCAGCG |
| | | CCCTCTAGTGTGGGATCTGGTACAGTGGCTGCAGGCGGTGGCGCACCAATGGCAGACA |
| | | ATAACGAAGGTGCCGACGGAGTGGGTAATGCCTCAGGAAATTGGCATTGCGATTCCACA |
| | | TGGCTGGGCGACAGAGTCATCACCACCAGCACCAGAACCTGGGCCCTGCCCACTTACAA |
| | | CAACCATCTCTACAAGCAAATCTCCAGCCAATCAGGAGCTTCAAACGACAACCACTACTT |
| | | CGGCTACAGCACCCCCTGGGGGTATTTTGACTTTAACAGATTCCACTGCCACTTTTCACC |
| | | ACGTGACTGGCAGCGACTCATCAACAACAACTGGGGATTCCGGCCCAAGAGACTCAGCT |
| | | TCAAGCTCTTCAACATCCAAGTCAAGGAGGTCACGACGAATGATGGCGTCACGACCATC |
| | | GCTAATAACCTTACCAGCACGGTTCAAGTCTTCTCGGACTCGGAGTACCAGCTTCCGTAC |
| | | GTCCTCGGCTCTGCGCACCAGGGCTGCCTCCCTCCGTTCCCGGCGGACGTGTTCATGAT |
| | | TCCGCAGTACGGCTACCTAACGCTCAACAATGGCAGCCAGGCAGTGGGACGGTCATCCT |
| | | TTTACTGCCTGGAATATTTTCCATCTCAAATGCTGCGAACTGGAAACAATTTTGAATTCAG |
| | | CTACACCTTCGAGGACGTGCCTTTCCACAGCAGCTACGCACACAGCCAGAGCTTGGACC |
| | | GACTGATGAATCCTCTCATTGACCAGTACCTGTACTACTTATCCAGAACTCAGTCCACAG |
| | | GAGGAACTCAAGGTACCCAGCAATTGTTATTTTCTCAAGCTGGGCCTGCAAACATGTCG |
| | | GCTCAGGCCAAGAACTGGCTGCCTGGACCTTGCTACCGGCAGCAGCGAGTCTCCACGA |
| | | CACTGTCGCAAAACAACAACAGCAACTTTGCTTGGACTGGTGCCACCAAATATCACCTGA |
| | | ACGGCAGAAACTCGTTGGTTAATCCCGGCGTCGCCATGGCAACTCACAAGGACGACGA |
| | | GGACCGCTTTTTCCCATCCAGCGGAGTCCTGATTTTTGGAAAAACTGGAGCAACTAACAA |
| | | AACTACATTGGAAAATGTGTTAATGACAAATGAAGAAGAAATTCGTCCTACTAATCCTGT |
| | | AGCCACGGAAGAATACGGGATAGTCAGCAGCAACTTACAAAACAGCAATACTGCGCCGG |
| | | AAATCGCAAACGTCAACAACCAGGGAGCCTTACCTGGCATGGTCTGGCAGAACCGGGA |
| | | CGTGTACCTGCAGGGTCCCATCTGGGCCAAGATTCCTCACACGGATGGCAACTTTCACC |
| | | CGTCTCCTTTGATGGGCGGCTTTGGACTTAAACATCCGCCTCCTCAGATCCTGATCAAGA |
| | | ACACTCCCGTTCCCGCTAATCCTCCGGAGGTGTTTACTCCTGCCAAGTTTGCTTCGTTCA |
| | | TCACACAGTACAGCACCGGACAAGTCAGCGTGGAAATCGAGTGGGAGCTGCAGAAGGA |
| | | AAACAGCAAGCGCTGGAACCCGGAGATTCAGTACACTTCAAACTACAACAAGTCTGTTA |
| | | GTGTGGACTTTACTGTAGACACTAATGGCGTGTATTCAGAGCCTCGCCCCATTGGCACC |
| | | AGATACCTGACTCGTAATCTGTAA |
| 58 | AAV | ATGGCTGCCGATGGTTATCTTCCAGATTGGCTCGAGGACACTCTCTCTGAAGGCATTCG |
| | C11.12.253 | CGAGTGGTGGGCGCTGAAACCTGGAGCTCCACAACCCAAGGCCAACCAACAGCATCAG |
| | R4.6 | GACAACGGCAGGGGTCTTGTGCTTCCTGGGTACAAGTACCTCGGACCCTTCAACGGACT |
| | | CGACAAGGGAGAGCCGGTCAACGAGGCAGACGCCGCGGCCCTCGAGCACGACAAGGC |
| | | CTACGACAAGCAGCTCGAGCAGGGGGACAACCCGTACCTCAAGTACAACCACGCCGAC |
| | | GCCGAGTTTCAGGAGCGTCTTCAAGAAGATACGTCTTTTGGGGGCAACCTTGGCAGAGC |
| | | AGTCTTCCAGGCCAAAAAGAGGATCCTTGAGCCTCTTGGTCTGGTTGAGGAAGCTGCTA |
| | | AGACGGCTCCTGGAAAGAAGAGACCGGTAGAACCGTCACCTCAGCGTTCCCCCGACTCC |
| | | TCCACGGGCATCGGCAAGAAAGGCCAGCAGCCCGCCAGAAAGAGACTCAATTTCGGTC |
| | | AGACTGGCGACTCAGAGTCAGTCCCCGACCCTCAACCTCTCGGAGAACCTCCAGCAGCG |
| | | CCCTCTAGTGTGGGATCTGGTACAGTGGCTGCAGGCGGTGGCGCACCAATGGCAGACA |
| | | ATAACGAAGGTGCCGACGGAGTGGGTAATGCCTCAGGAAATTGGCATTGCGATTCCACA |
| | | TGGCTGGGCGACAGAGTCATCACCACCAGCACCAGAACCTGGGCCCTGCCCACTTACAA |
| | | CAACCATCTCTACAAGCAAATCTCCAGCCAATCAGGAGCTTCAAACGACAACCACTACTT |
| | | CGGCTACAGCACCCCCTGGGGGTATTTTGACTTTAACAGATTCCACTGCCACTTTTCACC |
| | | ACGTGACTGGCAGCGACTCATCAACAACAACTGGGGATTCCGGCCCAAGAGACTCAGCT |
| | | TCAAGCTCTTCAACATCCAAGTCAAGGAGGTCACGACGAATGATGGCGTCACGACCATC |
| | | GCTAATAACCTTACCAGCACGGTTCAAGTCTTCTCGGACTCGGAGTACCAGCTTCCGTAC |
| | | GTCCTCGGCTCTGCGCACCAGGGCTGCCTCCCTCCGTTCCCGGCGGACGTGTTCATGAT |
| | | TCCGCAGTACGGCTACCTAACGCTCAACAATGGCAGCCAGGCAGTGGGACGGTCATCCT |
| | | TTTACTGCCTGGAATATTTTCCATCTCAAATGCTGCGAACTGGAAACAATTTTGAATTCAG |
| | | CTACACCTTCGAGGACGTGCCTTTCCACAGCAGCTACGCACACAGCCAGAGCTTGGACC |
| | | GACTGATGAATCCTCTCATTGACCAGTACCTGTACTACTTATCCAGAACTCAGTCCACAG |
| | | GAGGAACTCAAGGTACCCAGCAATTGTTATTTTCTCAAGCTGGGCCTGCAAACATGTCG |
| | | GCTCAGGCCAAGAACTGGCTGCCTGGACCTTGCTACCGGCAGCAGCGAGTCTCCACGA |
| | | CACTGTCGCAAAACAACAACAGCAACTTTGCTTGGACTGGTGCCACCAAATATCACCTGA |
| | | ACGGCAGAAACTCGTTGGTTAATCCCGGCGTCGCCATGGCAACTCACAAGGACGACGA |
| | | GGACCGCTTTTTCCCATCCAGCGGGGTACTAATCTTCGGTAAACAGGGCTCGGCCAACG |
| | | ACGCGACCCACGAGGACGTGATGCTCACAGACGAAGAAGAAATTCGTCCTACTAATCCT |
| | | GTAGCCACGGAAGAATACGGGATAGTCAGCAGCAACTTACAAAACAGCAATACTGCGCC |
| | | GGAAATCGCAAACGTCAACAACCAGGGAGCCTTACCTGGCATGGTCTGGCAGAACCGG |
| | | GACGTGTACCTGCAGGGTCCCATCTGGGCCAAGATTCCTCACACGGATGGCAACTTTCA |
| | | CCCGTCTCCTTTGATGGGCGGCTTTGGACTTAAACATCCGCCTCCTCAGATCCTGATCAA |
| | | GAACACTCCCGTTCCCGCTAATCCTCCGGAGGTGTTTACTCCTGCCAAGTTTGCTTCGTT |
| | | CATCACACAGTACAGCACCGGACAAGTCAGCGTGGAAATCGAGTGGGAGCTGCAGAAG |
| | | GAAAACAGCAAGCGCTGGAACCCGGAGATTCAGTACACTTCAAACTACAACAAGTCTGT |
| | | TAGTGTGGACTTTACTGTAGACACTAATGGCGTGTATTCAGAGCCTCGCCCCATTGGCAC |
| | | CAGATACCTGACTCGTAATCTGTAA |
| 59 | AAV | ATGGCTGCCGATGGTTATCTTCCAGATTGGCTCGAGGACACTCTCTCTGAAGGCATTCG |
| | C11.12.253 | CGAGTGGTGGGCGCTGAAACCTGGAGCTCCACAACCCAAGGCCAACCAACAGCATCAG |
| | R4.9 | GACAACGGCAGGGGTCTTGTGCTTCCTGGGTACAAGTACCTCGGACCCTTCAACGGACT |
| | | CGACAAGGGAGAGCCGGTCAACGAGGCAGACGCCGCGGCCCTCGAGCACGACAAGGC |
| | | CTACGACAAGCAGCTCGAGCAGGGGGACAACCCGTACCTCAAGTACAACCACGCCGAC |
| | | GCCGAGTTTCAGGAGCGTCTTCAAGAAGATACGTCTTTTGGGGGCAACCTTGGCAGAGC |
| | | AGTCTTCCAGGCCAAAAAGAGGATCCTTGAGCCTCTTGGTCTGGTTGAGGAAGCTGCTA |
| | | AGACGGCTCCTGGAAAGAAGAGACCGGTAGAACCGTCACCTCAGCGTTCCCCCGACTCC |
| | | TCCACGGGCATCGGCAAGAAAGGCCAGCAGCCCGCCAGAAAGAGACTCAATTTCGGTC |
| | | AGACTGGCGACTCAGAGTCAGTCCCCGACCCTCAACCTCTCGGAGAACCTCCAGCAGCG |
| | | CCCTCTAGTGTGGGATCTGGTACAGTGGCTGCAGGCGGTGGCGCACCAATGGCAGACA |
| | | ATAACGAAGGTGCCGACGGAGTGGGTAATGCCTCAGGAAATTGGCATTGCGATTCCACA |
| | | TGGCTGGGCGACAGAGTCATCACCACCAGCACCAGAACCTGGGCCCTGCCCACTTACAA |
| | | CAACCATCTCTACAAGCAAATCTCCAGCCAATCAGGAGCTTCAAACGACAACCACTACTT |
| | | CGGCTACAGCACCCCCTGGGGGTATTTTGACTTTAACAGATTCCACTGCCACTTTTCACC |
| | | ACGTGACTGGCAGCGACTCATCAACAACAACTGGGGATTCCGGCCCAAGAGACTCAGCT |
| | | TCAAGCTCTTCAACATCCAAGTCAAGGAGGTCACGACGAATGATGGCGTCACGACCATC |
| | | GCTAATAACCTTACCAGCACGGTTCAAGTCTTCTCGGACTCGGAGTACCAGCTTCCGTAC |
| | | GTCCTCGGCTCTGCGCACCAGGGCTGCCTCCCTCCGTTCCCGGCGGACGTGTTCATGAT |
| | | TCCGCAGTACGGCTACCTAACGCTCAACAATGGCAGCCAGGCAGTGGGACGGTCATCCT |
| | | TTTACTGCCTGGAATATTTTCCATCTCAAATGCTGCGAACTGGAAACAATTTTGAATTCAG |
| | | CTACACCTTCGAGGACGTGCCTTTCCACAGCAGCTACGCACACAGCCAGAGCTTGGACC |
| | | GACTGATGAATCCTCTCATTGACCAGTACCTGTACTACTTATCCAGAACTCAGTCCACAG |
| | | GAGGAACTCAAGGTACCCAGCAATTGTTATTTTCTCAAGCTGGGCCTGCAAACATGTCG |
| | | GCTCAGGCCAAGAACTGGCTGCCTGGACCTTGCTACCGGCAGCAGCGAGTCTCCACGA |
| | | CACTGTCGCAAAACAACAACAGCAACTTTGCTTGGACTGGTGCCACCAAATATCACCTGA |
| | | ACGGCAGAAACTCGTTGGTTAATCCCGGCGTCGCCATGGCAACTCACAAGGACGACGA |
| | | GGACCGCTTTTTCCCATCCAGCGGAGTTCTAATCTTTGGTAAGAAGAACACGAACACGA |
| | | ACAACGTGAACTACGAGAACGTGATGATGACAAACGAAGAAGAAATTCGTCCTACTAAT |
| | | CCTGTAGCCACGGAAGAATACGGGATAGTCAGCAGCAACTTACAAGCGGCTAATACTGC |
| | | AGCCCAGACACAAGTTGTCAACAACCAGGGAGCCTTACCTGGCATGGTCTGGCAGAACC |
| | | GGGACGTGTACCTGCAGGGTCCCATCTGGGCCAAGATTCCTCACACGGATGGCAACTTT |
| | | CACCCGTCTCCTTTGATGGGCGGCTTTGGACTTAAACATCCGCCTCCTCAGATCCTGATC |
| | | AAGAACACTCCCGTTCCCGCTAATCCTCCGGAGGTGTTTACTCCTGCCAAGTTTGCTTCG |
| | | TTCATCACACAGTACAGCACCGGACAAGTCAGCGTGGAAATCGAGTGGGAGCTGCAGA |
| | | AGGAAAACAGCAAGCGCTGGAACCCGGAGATTCAGTACACTTCAAACTACAACAAGTCT |
| | | GTTAGTGTGGACTTTACTGTAGACACTAATGGCGTGTATTCAGAGCCTCGCCCCATTGGC |
| | | ACCAGATACCTGACTCGTAATCTGTAA |
| 60 | AAV | ATGGCTGCCGATGGTTATCTTCCAGATTGGCTCGAGGACACTCTCTCTGAAGGCATTCG |
| | C11.12.269 | CGAGTGGTGGGCGCTGAAACCTGGAGCTCCACAACCCAAGGCCAACCAACAGCATCAG |
| | R4.1 | GACAACGGCAGGGGTCTTGTGCTTCCTGGGTACAAGTACCTCGGACCCTTCAACGGACT |
| | | CGACAAGGGAGAGCCGGTCAACGAGGCAGACGCCGCGGCCCTCGAGCACGACAAGGC |
| | | CTACGACAAGCAGCTCGAGCAGGGGGACAACCCGTACCTCAAGTACAACCACGCCGAC |
| | | GCCGAGTTTCAGGAGCGTCTTCAAGAAGATACGTCTTTTGGGGGCAACCTTGGCAGAGC |
| | | AGTCTTCCAGGCCAAAAAGAGGATCCTTGAGCCTCTTGGTCTGGTTGAGGAAGCTGCTA |
| | | AGACGGCTCCTGGAAAGAAGAGACCGGTAGAACCGTCACCTCAGCGTTCCCCCGACTCC |
| | | TCCACGGGCATCGGCAAGAAAGGCCAGCAGCCCGCCAGAAAGAGACTCAATTTCGGTC |
| | | AGACTGGCGACTCAGAGTCAGTCCCCGACCCTCAACCTCTCGGAGAACCTCCAGCAGCG |
| | | CCCTCTAGTGTGGGATCTGGTACAGTGGCTGCAGGCGGTGGCGCACCAATGGCAGACA |
| | | ATAACGAAGGTGCCGACGGAGTGGGTAATGCCTCAGGAAATTGGCATTGCGATTCCACA |
| | | TGGCTGGGCGACAGAGTCATCACCACCAGCACCAGAACCTGGGCCCTGCCCACTTACAA |
| | | CAACCATCTCTACAAGCAAATCTCCAGCCAATCAGGAGCTTCAAACGACAACCACTACTT |
| | | CGGCTACAGCACCCCCTGGGGGTATTTTGACTTTAACAGATTCCACTGCCACTTTTCACC |
| | | ACGTGACTGGCAGCGACTCATCAACAACAACTGGGGATTCCGGCCCAAGAGACTCAGCT |
| | | TCAAGCTCTTCAACATCCAAGTCAAGGAGGTCACGACGAATGATGGCGTCACGACCATC |
| | | GCTAATAACCTTACCAGCACGGTTCAAGTCTTCTCGGACTCGGAGTACCAGCTTCCGTAC |
| | | GTCCTCGGCTCTGCGCACCAGGGCTGCCTCCCTCCGTTCCCGGCGGACGTGTTCATGAT |
| | | TCCGCAGTACGGCTACCTAACGCTCAACAATGGCAGCCAGGCAGTGGGACGGTCATCCT |
| | | TTTACTGCCTGGAATATTTTCCATCTCAAATGCTGCGAACTGGAAACAATTTTGAATTCAG |
| | | CTACACCTTCGAGGACGTGCCTTTCCACAGCAGCTACGCACACAGCCAGAGCTTGGACC |
| | | GACTGATGAATCCTCTCATTGACCAATATCTATACTATTTAGGCAGGACTCAGGCGACCC |
| | | CGGGCGGAACTACGGGGCAGAACACCTTGTTATTTTCTCAAGCTGGGCCTGCAAACATG |
| | | TCGGCTCAGGCCAAGAACTGGCTGCCTGGACCTTGCTACCGGCAGCAGCGAGTCTCCA |
| | | CGACACTGTCGCAAAACAACAACAGCAACTTTGCTTGGACTGGTGCCACCAAATATCACC |
| | | TGAACGGCAGAAACTCGTTGGTTAATCCCGGCGTCGCCATGGCAACTCACAAGGACGAC |
| | | GAGGACCGCTTTTTCCCATCCAGCGGAGTCCTGATTTTTGGAAAAACTGGAGCAACTAA |
| | | CAAAACTACATTGGAAAATGTGTTAATGACAAATGAAGAAGAAATTCGTCCTACTAATCC |
| | | TGTAGCCACGGAAGAATACGGGATAGTCAGCAGCAACTTACAAGCGGCTAATACTGCAG |
| | | CCCAGACACAAGTTGTCAACAACCAGGGAGCCTTACCTGGCATGGTCTGGCAGAACCGG |
| | | GACGTGTACCTGCAGGGTCCCATCTGGGCCAAGATTCCTCACACGGATGGCAACTTTCA |
| | | CCCGTCTCCTTTGATGGGCGGCTTTGGACTTAAACATCCGCCTCCTCAGATCCTGATCAA |
| | | GAACACTCCCGTTCCCGCTAATCCTCCGGAGGTGTTTACTCCTGCCAAGTTTGCTTCGTT |
| | | CATCACACAGTACAGCACCGGACAAGTCAGCGTGGAAATCGAGTGGGAGCTGCAGAAG |
| | | GAAAACAGCAAGCGCTGGAACCCGGAGATTCAGTACACTTCAAACTACAACAAGTCTGT |
| | | TAGTGTGGACTTTACTGTAGACACTAATGGCGTGTATTCAGAGCCTCGCCCCATTGGCAC |
| | | CAGATACCTGACTCGTAATCTGTAA |
| 61 | AAV | ATGGCTGCCGATGGTTATCTTCCAGATTGGCTCGAGGACACTCTCTCTGAAGGCATTCG |
| | C11.12.269 | CGAGTGGTGGGCGCTGAAACCTGGAGCTCCACAACCCAAGGCCAACCAACAGCATCAG |
| | R4.3 | GACAACGGCAGGGGTCTTGTGCTTCCTGGGTACAAGTACCTCGGACCCTTCAACGGACT |
| | | CGACAAGGGAGAGCCGGTCAACGAGGCAGACGCCGCGGCCCTCGAGCACGACAAGGC |
| | | CTACGACAAGCAGCTCGAGCAGGGGGACAACCCGTACCTCAAGTACAACCACGCCGAC |
| | | GCCGAGTTTCAGGAGCGTCTTCAAGAAGATACGTCTTTTGGGGGCAACCTTGGCAGAGC |
| | | AGTCTTCCAGGCCAAAAAGAGGATCCTTGAGCCTCTTGGTCTGGTTGAGGAAGCTGCTA |
| | | AGACGGCTCCTGGAAAGAAGAGACCGGTAGAACCGTCACCTCAGCGTTCCCCCGACTCC |
| | | TCCACGGGCATCGGCAAGAAAGGCCAGCAGCCCGCCAGAAAGAGACTCAATTTCGGTC |
| | | AGACTGGCGACTCAGAGTCAGTCCCCGACCCTCAACCTCTCGGAGAACCTCCAGCAGCG |
| | | CCCTCTAGTGTGGGATCTGGTACAGTGGCTGCAGGCGGTGGCGCACCAATGGCAGACA |
| | | ATAACGAAGGTGCCGACGGAGTGGGTAATGCCTCAGGAAATTGGCATTGCGATTCCACA |
| | | TGGCTGGGCGACAGAGTCATCACCACCAGCACCAGAACCTGGGCCCTGCCCACTTACAA |
| | | CAACCATCTCTACAAGCAAATCTCCAGCCAATCAGGAGCTTCAAACGACAACCACTACTT |
| | | CGGCTACAGCACCCCCTGGGGGTATTTTGACTTTAACAGATTCCACTGCCACTTTTCACC |
| | | ACGTGACTGGCAGCGACTCATCAACAACAACTGGGGATTCCGGCCCAAGAGACTCAGCT |
| | | TCAAGCTCTTCAACATCCAAGTCAAGGAGGTCACGACGAATGATGGCGTCACGACCATC |
| | | GCTAATAACCTTACCAGCACGGTTCAAGTCTTCTCGGACTCGGAGTACCAGCTTCCGTAC |
| | | GTCCTCGGCTCTGCGCACCAGGGCTGCCTCCCTCCGTTCCCGGCGGACGTGTTCATGAT |
| | | TCCGCAGTACGGCTACCTAACGCTCAACAATGGCAGCCAGGCAGTGGGACGGTCATCCT |
| | | TTTACTGCCTGGAATATTTTCCATCTCAAATGCTGCGAACTGGAAACAATTTTGAATTCAG |
| | | CTACACCTTCGAGGACGTGCCTTTCCACAGCAGCTACGCACACAGCCAGAGCTTGGACC |
| | | GACTGATGAATCCTCTCATTGACCAGTACCTGTACTACTTATCCAGAACTCAGTCCACAG |
| | | GAGGAACTCAAGGTACCCAGCAATTGTTATTTTCTCAAGCTGGGCCTGCAAACATGTCG |
| | | GCTCAGGCCAAGAACTGGCTGCCTGGACCTTGCTACCGGCAGCAGCGAGTCTCCACGA |
| | | CACTGTCGCAAAACAACAACAGCAACTTTGCTTGGACTGGTGCCACCAAATATCACCTGA |
| | | ACGGCAGAAACTCGTTGGTTAATCCCGGCGTCGCCATGGCAACTCACAAGGACGACGA |
| | | GGACCGCTTTTTCCCATCCAGCGGAGTCCTGATTTTTGGAAAAACTGGAGCAACTAACAA |
| | | AACTACATTGGAAAATGTGTTAATGACAAATGAAGAAGAAATTCGTCCTACTAATCCTGT |
| | | AGCCACGGAAGAATACGGGATAGTCAGCAGCAACTTACAAAAGGAGAATACTCCGGCG |
| | | CAAATCGAAGCCGTCAACAACCAGGGAGCCTTACCTGGCATGGTCTGGCAGAACCGGG |
| | | ACGTGTACCTGCAGGGTCCCATCTGGGCCAAGATTCCTCACACGGATGGCAACTTTCAC |
| | | CCGTCTCCTTTGATGGGCGGCTTTGGACTTAAACATCCGCCTCCTCAGATCCTGATCAAG |
| | | AACACTCCCGTTCCCGCTAATCCTCCGGAGGTGTTTACTCCTGCCAAGTTTGCTTCGTTC |
| | | ATCACACAGTACAGCACCGGACAAGTCAGCGTGGAAATCGAGTGGGAGCTGCAGAAGG |
| | | AAAACAGCAAGCGCTGGAACCCGGAGATTCAGTACACTTCAAACTACAACAAGTCTGTT |
| | | AGTGTGGACTTTACTGTAGACACTAATGGCGTGTATTCAGAGCCTCGCCCCATTGGCAC |
| | | CAGATACCTGACTCGTAATCTGTAA |
| 62 | AAV | ATGGCTGCCGATGGTTATCTTCCAGATTGGCTCGAGGACACTCTCTCTGAAGGCATTCG |
| | C11.12.269 | CGAGTGGTGGGCGCTGAAACCTGGAGCTCCACAACCCAAGGCCAACCAACAGCATCAG |
| | R4.5 | GACAACGGCAGGGGTCTTGTGCTTCCTGGGTACAAGTACCTCGGACCCTTCAACGGACT |
| | | CGACAAGGGAGAGCCGGTCAACGAGGCAGACGCCGCGGCCCTCGAGCACGACAAGGC |
| | | CTACGACAAGCAGCTCGAGCAGGGGGACAACCCGTACCTCAAGTACAACCACGCCGAC |
| | | GCCGAGTTTCAGGAGCGTCTTCAAGAAGATACGTCTTTTGGGGGCAACCTTGGCAGAGC |
| | | AGTCTTCCAGGCCAAAAAGAGGATCCTTGAGCCTCTTGGTCTGGTTGAGGAAGCTGCTA |
| | | AGACGGCTCCTGGAAAGAAGAGACCGGTAGAACCGTCACCTCAGCGTTCCCCCGACTCC |
| | | TCCACGGGCATCGGCAAGAAAGGCCAGCAGCCCGCCAGAAAGAGACTCAATTTCGGTC |
| | | AGACTGGCGACTCAGAGTCAGTCCCCGACCCTCAACCTCTCGGAGAACCTCCAGCAGCG |
| | | CCCTCTAGTGTGGGATCTGGTACAGTGGCTGCAGGCGGTGGCGCACCAATGGCAGACA |
| | | ATAACGAAGGTGCCGACGGAGTGGGTAATGCCTCAGGAAATTGGCATTGCGATTCCACA |
| | | TGGCTGGGCGACAGAGTCATCACCACCAGCACCAGAACCTGGGCCCTGCCCACTTACAA |
| | | CAACCATCTCTACAAGCAAATCTCCAGCCAATCAGGAGCTTCAAACGACAACCACTACTT |
| | | CGGCTACAGCACCCCCTGGGGGTATTTTGACTTTAACAGATTCCACTGCCACTTTTCACC |
| | | ACGTGACTGGCAGCGACTCATCAACAACAACTGGGGATTCCGGCCCAAGAGACTCAGCT |
| | | TCAAGCTCTTCAACATCCAAGTCAAGGAGGTCACGACGAATGATGGCGTCACGACCATC |
| | | GCTAATAACCTTACCAGCACGGTTCAAGTCTTCTCGGACTCGGAGTACCAGCTTCCGTAC |
| | | GTCCTCGGCTCTGCGCACCAGGGCTGCCTCCCTCCGTTCCCGGCGGACGTGTTCATGAT |
| | | TCCGCAGTACGGCTACCTAACGCTCAACAATGGCAGCCAGGCAGTGGGACGGTCATCCT |
| | | TTTACTGCCTGGAATATTTTCCATCTCAAATGCTGCGAACTGGAAACAATTITGAATTCAG |
| | | CTACACCTTCGAGGACGTGCCTTTCCACAGCAGCTACGCACACAGCCAGAGCTTGGACC |
| | | GACTGATGAATCCTCTCATTGACCAGTACCTGTACTACTTATCCAGAACTCAGTCCACAG |
| | | GAGGAACTCAAGGTACCCAGCAATTGTTATTTTCTCAAGCTGGGCCTGCAAACATGTCG |
| | | GCTCAGGCCAAGAACTGGCTGCCTGGACCTTGCTACCGGCAGCAGCGAGTCTCCACGA |
| | | CACTGTCGCAAAACAACAACAGCAACTTTGCTTGGACTGGTGCCACCAAATATCACCTGA |
| | | ACGGCAGAAACTCGTTGGTTAATCCCGGCGTCGCCATGGCAACTCACAAGGACGACGA |
| | | GGACCGCTTTTTCCCATCCAGCGGGGTCCTGATATTTGGTAAGCAGAGCTCGAGCGGGG |
| | | CCAAGGTGAACCTCGAGGGGGTGTTGCTCACAGGCGAAGAAGAAATTCGTCCTACTAAT |
| | | CCTGTAGCCACGGAAGAATACGGGATAGTCAGCAGCAACTTACAAGCGGCTAATACTGC |
| | | AGCCCAGACACAAGTTGTCAACAACCAGGGAGCCTTACCTGGCATGGTCTGGCAGAACC |
| | | GGGACGTGTACCTGCAGGGTCCCATCTGGGCCAAGATTCCTCACACGGATGGCAACTTT |
| | | CACCCGTCTCCTTTGATGGGCGGCTTTGGACTTAAACATCCGCCTCCTCAGATCCTGATC |
| | | AAGAACACTCCCGTTCCCGCTAATCCTCCGGAGGTGTTTACTCCTGCCAAGTTTGCTTCG |
| | | TTCATCACACAGTACAGCACCGGACAAGTCAGCGTGGAAATCGAGTGGGAGCTGCAGA |
| | | AGGAAAACAGCAAGCGCTGGAACCCGGAGATTCAGTACACTTCAAACTACAACAAGTCT |
| | | GTTAGTGTGGACTTTACTGTAGACACTAATGGCGTGTATTCAGAGCCTCGCCCCATTGGC |
| | | ACCAGATACCTGACTCGTAATCTGTAA |
| 63 | C11.12.269 | ATGGCTGCCGATGGTTATCTTCCAGATTGGCTCGAGGACACTCTCTCTGAAGGCATTCG |
| | R4.6 | CGAGTGGTGGGCGCTGAAACCTGGAGCTCCACAACCCAAGGCCAACCAACAGCATCAG |
| | | GACAACGGCAGGGGTCTTGTGCTTCCTGGGTACAAGTACCTCGGACCCTTCAACGGACT |
| | | CGACAAGGGAGAGCCGGTCAACGAGGCAGACGCCGCGGCCCTCGAGCACGACAAGGC |
| | | CTACGACAAGCAGCTCGAGCAGGGGGACAACCCGTACCTCAAGTACAACCACGCCGAC |
| | | GCCGAGTTTCAGGAGCGTCTTCAAGAAGATACGTCTTTTGGGGGCAACCTTGGCAGAGC |
| | | AGTCTTCCAGGCCAAAAAGAGGATCCTTGAGCCTCTTGGTCTGGTTGAGGAAGCTGCTA |
| | | AGACGGCTCCTGGAAAGAAGAGACCGGTAGAACCGTCACCTCAGCGTTCCCCCGACTCC |
| | | TCCACGGGCATCGGCAAGAAAGGCCAGCAGCCCGCCAGAAAGAGACTCAATTTCGGTC |
| | | AGACTGGCGACTCAGAGTCAGTCCCCGACCCTCAACCTCTCGGAGAACCTCCAGCAGCG |
| | | CCCTCTAGTGTGGGATCTGGTACAGTGGCTGCAGGCGGTGGCGCACCAATGGCAGACA |
| | | ATAACGAAGGTGCCGACGGAGTGGGTAATGCCTCAGGAAATTGGCATTGCGATTCCACA |
| | | TGGCTGGGCGACAGAGTCATCACCACCAGCACCAGAACCTGGGCCCTGCCCACTTACAA |
| | | CAACCATCTCTACAAGCAAATCTCCAGCCAATCAGGAGCTTCAAACGACAACCACTACTT |
| | | CGGCTACAGCACCCCCTGGGGGTATTTTGACTTTAACAGATTCCACTGCCACTTTTCACC |
| | | ACGTGACTGGCAGCGACTCATCAACAACAACTGGGGATTCCGGCCCAAGAGACTCAGCT |
| | | TCAAGCTCTTCAACATCCAAGTCAAGGAGGTCACGACGAATGATGGCGTCACGACCATC |
| | | GCTAATAACCTTACCAGCACGGTTCAAGTCTTCTCGGACTCGGAGTACCAGCTTCCGTAC |
| | | GTCCTCGGCTCTGCGCACCAGGGCTGCCTCCCTCCGTTCCCGGCGGACGTGTTCATGAT |
| | | TCCGCAGTACGGCTACCTAACGCTCAACAATGGCAGCCAGGCAGTGGGACGGTCATCCT |
| | | TTTACTGCCTGGAATATTTTCCATCTCAAATGCTGCGAACTGGAAACAATTTTGAATTCAG |
| | | CTACACCTTCGAGGACGTGCCTTTCCACAGCAGCTACGCACACAGCCAGAGCTTGGACC |
| | | GACTGATGAATCCTCTCATTGACCAGTACCTGTACTACTTAAGCAAGACTCAGGCGACCA |
| | | GCGGAACTAAGGACACCAACCAGTTGTTATTTTCTCAAGCTGGGCCTGCAAACATGTCG |
| | | GCTCAGGCCAAGAACTGGCTGCCTGGACCTTGCTACCGGCAGCAGCGAGTCTCCACGA |
| | | CACTGTCGCAAAACAACAACAGCAACTTTGCTTGGACTGGTGCCACCAAATATCACCTGA |
| | | ACGGCAGAAACTCGTTGGTTAATCCCGGCGTCGCCATGGCAACTCACAAGGACGACGA |
| | | GGACCGCTTTTTCCCATCCAGCGGAGTCCTGATTTTTGGAAAAACTGGAGCAACTAACAA |
| | | AACTACATTGGAAAATGTGTTAATGACAAATGAAGAAGAAATTCGTCCTACTAATCCTGT |
| | | AGCCACGGAAGAATACGGGATAGTCAGCAGCAACTTACAAGCGGCTAATACTGCAGCCC |
| | | AGACACAAGTTGTCAACAACCAGGGAGCCTTACCTGGCATGGTCTGGCAGAACCGGGA |
| | | CGTGTACCTGCAGGGTCCCATCTGGGCCAAGATTCCTCACACGGATGGCAACTTTCACC |
| | | CGTCTCCTTTGATGGGCGGCTTTGGACTTAAACATCCGCCTCCTCAGATCCTGATCAAGA |
| | | ACACTCCCGTTCCCGCTAATCCTCCGGAGGTGTTTACTCCTGCCAAGTTTGCTTCGTTCA |
| | | TCACACAGTACAGCACCGGACAAGTCAGCGTGGAAATCGAGTGGGAGCTGCAGAAGGA |
| | | AAACAGCAAGCGCTGGAACCCGGAGATTCAGTACACTTCAAACTACAACAAGTCTGTTA |
| | | GTGTGGACTTTACTGTAGACACTAATGGCGTGTATTCAGAGCCTCGCCCCATTGGCACC |
| | | AGATACCTGACTCGTAATCTGTAA |
| 64 | AAV | ATGGCTGCCGATGGTTATCTTCCAGATTGGCTCGAGGACACTCTCTCTGAAGGCATTCG |
| | C11.12.269 | CGAGTGGTGGGCGCTGAAACCTGGAGCTCCACAACCCAAGGCCAACCAACAGCATCAG |
| | R4.20 | GACAACGGCAGGGGTCTTGTGCTTCCTGGGTACAAGTACCTCGGACCCTTCAACGGACT |
| | | CGACAAGGGAGAGCCGGTCAACGAGGCAGACGCCGCGGCCCTCGAGCACGACAAGGC |
| | | CTACGACAAGCAGCTCGAGCAGGGGGACAACCCGTACCTCAAGTACAACCACGCCGAC |
| | | GCCGAGTTTCAGGAGCGTCTTCAAGAAGATACGTCTTTTGGGGGCAACCTTGGCAGAGC |
| | | AGTCTTCCAGGCCAAAAAGAGGATCCTTGAGCCTCTTGGTCTGGTTGAGGAAGCTGCTA |
| | | AGACGGCTCCTGGAAAGAAGAGACCGGTAGAACCGTCACCTCAGCGTTCCCCCGACTCC |
| | | TCCACGGGCATCGGCAAGAAAGGCCAGCAGCCCGCCAGAAAGAGACTCAATTTCGGTC |
| | | AGACTGGCGACTCAGAGTCAGTCCCCGACCCTCAACCTCTCGGAGAACCTCCAGCAGCG |
| | | CCCTCTAGTGTGGGATCTGGTACAGTGGCTGCAGGCGGTGGCGCACCAATGGCAGACA |
| | | ATAACGAAGGTGCCGACGGAGTGGGTAATGCCTCAGGAAATTGGCATTGCGATTCCACA |
| | | TGGCTGGGCGACAGAGTCATCACCACCAGCACCAGAACCTGGGCCCTGCCCACTTACAA |
| | | CAACCATCTCTACAAGCAAATCTCCAGCCAATCAGGAGCTTCAAACGACAACCACTACTT |
| | | CGGCTACAGCACCCCCTGGGGGTATTTTGACTTTAACAGATTCCACTGCCACTTTTCACC |
| | | ACGTGACTGGCAGCGACTCATCAACAACAACTGGGGATTCCGGCCCAAGAGACTCAGCT |
| | | TCAAGCTCTTCAACATCCAAGTCAAGGAGGTCACGACGAATGATGGCGTCACGACCATC |
| | | GCTAATAACCTTACCAGCACGGTTCAAGTCTTCTCGGACTCGGAGTACCAGCTTCCGTAC |
| | | GTCCTCGGCTCTGCGCACCAGGGCTGCCTCCCTCCGTTCCCGGCGGACGTGTTCATGAT |
| | | TCCGCAGTACGGCTACCTAACGCTCAACAATGGCAGCCAGGCAGTGGGACGGTCATCCT |
| | | TTTACTGCCTGGAATATTTTCCATCTCAAATGCTGCGAACTGGAAACAATTTTGAATTCAG |
| | | CTACACCTTCGAGGACGTGCCTTTCCACAGCAGCTACGCACACAGCCAGAGCTTGGACC |
| | | GACTGATGAATCCTCTCATTGACCAGTACCTGTACTACTTATCCAGAACTCAGTCCACAG |
| | | GAGGAACTCAAGGTACCCAGCAATTGTTATTTTCTCAAGCTGGGCCTGCAAACATGTCG |
| | | GCTCAGGCCAAGAACTGGCTGCCTGGACCTTGCTACCGGCAGCAGCGAGTCTCCACGA |
| | | CACTGTCGCAAAACAACAACAGCAACTTTGCTTGGACTGGTGCCACCAAATATCACCTGA |
| | | ACGGCAGAAACTCGTTGGTTAATCCCGGCGTCGCCATGGCAACTCACAAGGACGACGA |
| | | GGACCGCTTTTTCCCATCCAGCGGAGTCCTGATTTTTGGAAAAACTGGAGCAACTAACAA |
| | | AACTACATTGGAAAATGTGTTAATGACAAATGAAGAAGAAATTCGTCCTACTAATCCTGT |
| | | AGCCACGGAAGAATACGGGATAGTCAGCAGCAACTTACAAACGGGGAATACTGCGCCG |
| | | GAAACCCTAATCGTCAACCACCAGGGAGCCTTACCTGGCATGGTCTGGCAGAACCGGGA |
| | | CGTGTACCTGCAGGGTCCCATCTGGGCCAAGATTCCTCACACGGATGGCAACTTTCACC |
| | | CGTCTCCTTTGATGGGCGGCTTTGGACTTAAACATCCGCCTCCTCAGATCCCTGATCAAGA |
| | | ACACTCCCGTTCCCGCTAATCCTCCGGAGGTGTTTACTCCTGCCAAGTTTGCTTCGTTCA |
| | | TCACACAGTACAGCACCGGACAAGTCAGCGTGGAAATCGAGTGGGAGCTGCAGAAGGA |
| | | AAACAGCAAGCGCTGGAACCCGGAGATTCAGTACACTTCAAACTACAACAAGTCTGTTA |
| | | GTGTGGACTTTACTGTAGACACTAATGGCGTGTATTCAGAGCCTCGCCCCATTGGCACC |
| | | AGATACCTGACTCGTAATCTGTAA |

1. A capsid polypeptide, comprising:
(i) the sequence of amino acids set forth in any one of SEQ ID NOs:5-8, or a sequence having at least or about 95%, 96%, 97%, 98% or 99% sequence identity thereto;
(ii) the sequence of amino acids at positions 138-735 of SEQ ID NO:5, positions 138-735 of SEQ ID NO:6, or positions 138-737 of SEQ ID NO:7 or 8; or a sequence having at least or about 95%, 96%, 97%, 98% or 99% sequence identity thereto; and/or
(iii) the sequence of amino acids at positions 204-735 of SEQ ID NO:5, positions 204-735 of SEQ ID NO:6, or positions 204-737 of SEQ ID NO: 7 or 8, or a sequence having at least or about 95%, 96%, 97%, 98% or 99% sequence identity thereto.

2-47. (canceled)
